SEQUENCE LISTING (14 sequences)

SEQ ID NO: 1 - RNA, 267 nt, synthetic construct
  source 1..267: mol_type = other RNA; note = 5' regulatory region of VEEV
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattcta

SEQ ID NO: 2 - RNA, 282 nt, synthetic construct
  source 1..282: mol_type = other RNA; note = 5' regulatory sequence VEEV modified
gatgggcggcgcataagagaagcccagaccaattacctacccaaaatcgagaaagttcacgttgacatcgaggaagacagcccattcctcagagctttgcagcggagcttccggcagtttgaggtagaagccaaggaggtgactgataatcaccatcctattgccagagcgttttcgcatctggcttcaaaactgatcgaaacggaggtggacccatccgacacgatccttgacattggaagtgcgcccgcccgcagaaagtattctactagtgccaccatg

SEQ ID NO: 3 - protein, 9 aa, synthetic construct
LYEKVRSQL

SEQ ID NO: 4 - protein, 9 aa, synthetic construct
CPHAGAKSF

SEQ ID NO: 5 - protein, 9 aa, synthetic construct
VEPGDKITF

SEQ ID NO: 6 - protein, 10 aa, synthetic construct
SYINDKGKEV

SEQ ID NO: 7 - protein, 8 aa, synthetic construct
FYHKCDNT

SEQ ID NO: 8 - protein, 9 aa, synthetic construct
STASSWSYI

SEQ ID NO: 9 - protein, 15 aa, synthetic construct
STRIYQILAIYSTVA

SEQ ID NO: 10 - protein, 15 aa, synthetic construct
KYVKSTKLRLATGLR

SEQ ID NO: 11 - protein, 15 aa, synthetic construct
GKCPKYVKSTKLRLA

SEQ ID NO: 12 - protein, 15 aa, synthetic construct
AIYSTVASSLVLVVS

SEQ ID NO: 13 - protein, 15 aa, synthetic construct
EELREQLSSVSSFER

SEQ ID NO: 14 - protein, 15 aa, synthetic construct
VKLESTRIYQILAIY

(19) United States
(12) Patent Application Publication
(10) Pub. No.: US 2025/0250626 A1
(43) Pub. Date: Aug. 7, 2025

(54) METHODS FOR DETERMINING MUTATIONS FOR INCREASING MODIFIED REPLICABLE RNA FUNCTION AND RELATED COMPOSITIONS AND THEIR USE

(71) Applicants: BioNTech SE, Mainz (DE); TRON - Translationale Onkologie an der Universitätsmedizin der Johannes Gutenberg-Universität Mainz, Mainz (DE)

(72) Inventors: Tim BEISSERT, Mainz (DE); Mario PERKOVIC, Mainz (DE); Stefanie GAWLETTA, Mainz (DE); Patrick SORN, Mainz (DE); Silke BRILL, Mainz (DE); Evelin NETT, Mainz (DE)

(21) Appl. No.: 18/702,287

(22) PCT Filed: Oct. 17, 2022
(86) PCT No.: PCT/EP2022/078869

§ 371 Date: Apr. 17, 2024

(30) Foreign Application Priority Data
Oct. 18, 2021 (WO) PCT/EP2021/078828

(51) Int. Cl. C12Q 1/6869; A61K 39/00; A61K 39/145

(52) U.S. Cl. CPC C12Q 1/6869; A61K 39/0011; A61K 39/145; A61K 2039/53

(57) ABSTRACT

The present invention relates to methods for restoring or improving the ability of a modified nucleotide-containing replicable RNA to be replicated and/or translated. The method includes identifying nucleotide changes in the replicable RNA that compensate for the lowered ability of modified-nucleotide-containing replicable RNA molecules to replicate and/or be translated. The present invention also relates to modified nucleotide-containing replicable RNA molecules incorporating such identified nucleotide changes and the use of such replicable RNA molecules in therapy.

The present invention relates to methods for restoring or improving the ability of a modified nucleotide-containing replicable RNA to be replicated and/or translated. The method includes identifying nucleotide changes, e.g., in the replicable RNA that compensate for the lowered ability of modified-nucleotide-containing replicable RNA molecules to replicate and/or be translated. The present invention also relates to modified nucleotide-containing replicable RNA molecules incorporating such identified nucleotide changes and the use of such replicable RNA molecules in therapy.

### BACKGROUND

Recently, mRNA-based vaccines proved their immunogenicity in clinical studies to combat the Covid-19 epidemic. These RNA vaccines are highly effective and induce very strong T cell immune responses and high levels of neutralizing antibodies (Walsh et al., 2020, N Engl J Med 383:2439-2450; Sahin et al., 2020, Nature 586:594-599). The first two mRNA vaccines that obtained regulatory approval contain a chemically modified nucleotide, N1-methyl-pseudouridine (1m ψ), instead of uridine. This modification improves the translation of the mRNA in immune competent cells by largely avoiding the stimulation of innate immune pathways leading to interferon response (Andries et al., 2015, J Control Release 217:337-344).

These approved RNA vaccines require 30 to 100 μg RNA per dose, and two consecutive doses spaced by several weeks (prime-boost regimen). This culminates in 60 to 200 g RNA needed to immunize 1 million people. A dose reduction to less than 1 μg would therefore have great impact on the production time needed to supply the population with a vaccine against a novel pathogen.

A vaccine approach under investigation that promises to achieve a significant dose reduction is to use self-amplifying RNA (saRNA). saRNA can be engineered from alphaviral genomes by replacing alphaviral structural genes with antigens against which an immune response is desired. saRNA encodes the alphaviral replicase which harbors all enzymatic function to replicate the saRNA molecule, thus leading to an amplification of the input vaccine amount. Unfortunately, the innate immune response caused by saRNA strongly inhibits the effectiveness of the saRNA vaccine, which may be a reason why rather high amounts of saRNA were used in a preclinical study of a saRNA Covid-19 vaccine in non-human primates (Erasmus et al., 2020, Sci Transl Med 12: eabc9396).

Alphaviruses are typical representatives of positive-stranded RNA viruses. The hosts of alphaviruses include a wide range of organisms, comprising insects, fish and mammals, such as domesticated animals and humans. Alphaviruses replicate in the cytoplasm of infected cells (for review of the alphaviral life cycle see José et al., 2009, Future Microbiol. 4:837-856). The total genome length of many alphaviruses typically ranges from between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1.

In cells infected by an alphavirus, only the non-structural proteins are translated from the genomic RNA, while the structural proteins are translatable from a subgenomic transcript, which is an RNA molecule that resembles eukaryotic messenger RNA (mRNA; Gould et al., 2010, Antiviral Res. 87:111-124). Following infection, i.e., at early stages of the viral life cycle, the (+) stranded genomic RNA directly acts like a messenger RNA for the translation of the open reading frame encoding the non-structural poly-protein (nsP1234). In some alphaviruses, there is an opal stop codon between the coding sequences of nsP3 and nsP4: polyprotein P123, containing nsP1, nsP2, and nsP3, is produced when translation terminates at the opal stop codon, and polyprotein P1234, containing in addition nsP4, is produced upon readthrough of this opal codon (Strauss & Strauss, 1994, Microbiol. Rev. 58:491-562; Rupp et al., 2015, J. Gen. Virology 96:2483-2500). nsP1234 is autoproteolytically cleaved into the fragments nsP123 and nsP4. The polypeptides nsP123 and nsP4 associate to form the (−) strand replicase complex that transcribes (−) stranded RNA, using the (+) stranded genomic RNA as template. Typically, at later stages, the nsP123 fragment is completely cleaved into individual proteins nsP1, nsP2 and nsP3 (Shirako & Strauss, 1994, J. Virol. 68:1874-1885). All four proteins assemble to form the (+) strand replicase complex that synthesizes new (+) stranded genomes, using the (−) stranded complement of genomic RNA as template (Kim et al., 2004, Virology 323:153-163, Vasiljeva et al., 2003, J. Biol. Chem. 278:41636-41645).

In infected cells, subgenomic RNA as well as new genomic RNA is provided with a 5′-cap by nsP1 (Pettersson et al., 1980, Eur. J. Biochem. 105:435-443; Rozanov et al., 1992, J. Gen. Virology 73:2129-2134), and provided with a poly-adenylate [poly(A)] tail by nsP4 (Rubach et al., 2009, Virology 384:201-208). Thus, both subgenomic RNA and genomic RNA resemble messenger RNA (mRNA).

Alphavirus structural proteins (core nucleocapsid protein C, envelope protein E2 and envelope protein E1, all constituents of the virus particle) are typically encoded by one single open reading frame under control of a subgenomic promoter (Strauss & Strauss, 1994, Microbiol. Rev. 58:491-562). The subgenomic promoter is recognized by alphaviral non-structural proteins acting in cis. In particular, alphavirus replicase synthesizes a (+) stranded subgenomic transcript using the (−) stranded complement of genomic RNA as template. The (+) stranded subgenomic transcript encodes the alphavirus structural proteins (Kim et al., 2004, Virology 323:153-163, Vasiljeva et al., 2003, J. Biol. Chem. 278:41636-41645). The subgenomic RNA transcript serves as template for translation of the open reading frame encoding the structural proteins as one poly-protein, and the poly-protein is cleaved to yield the structural proteins. At a late stage of alphavirus infection in a host cell, a packaging signal which is located within the coding sequence of nsP2 ensures selective packaging of genomic RNA into budding virions, packaged by structural proteins (White et al., 1998, J. Virol. 72:4320-4326).

In infected cells, (−) strand RNA synthesis is typically observed only in the first 3-4 h post infection, and is undetectable at late stages, at which time the synthesis of only (+) strand RNA (both genomic and subgenomic) is observed. According to Frolov et al., 2001, RNA 7:1638-1651, the prevailing model for regulation of RNA synthesis suggests a dependence on the processing of the non-structural poly-protein: initial cleavage of the non-structural polyprotein nsP1234 yields nsP123 and nsP4; nsP4 acts as RNA-dependent RNA polymerase (RdRp) that is active for (−) strand synthesis, but inefficient for the generation of (+) strand RNAs. Further processing of the polyprotein nsP123, including cleavage at the nsP2/nsP3 junction, changes the template specificity of the replicase to increase synthesis of (+) strand RNA and to decrease or terminate synthesis of (−) strand RNA.

The synthesis of alphaviral RNA is also regulated by cis-acting RNA elements, including four conserved sequence elements (CSEs; Strauss & Strauss, 1994, Microbiol. Rev. 58:491-562; and Frolov, 2001, RNA 7:1638-1651). The alphavirus genome comprises four conserved sequence elements (CSEs) which are understood to be important for viral RNA replication in the host cell. CSE 1, found at or near the 5′ end of the virus genome, is believed to function as a promoter for (+) strand synthesis from (−) strand templates. CSE 2, located downstream of CSE 1 but still close to the 5′ end of the genome within the coding sequence for nsP1 is thought to act as a promoter for initiation of (−) strand synthesis from a genomic RNA template (note that the subgenomic RNA transcript, which does not comprise CSE 2, does not function as a template for (−) strand synthesis). CSE 3 is located in the junction region between the coding sequence for the non-structural and structural proteins and acts as core promoter for the efficient transcription of the subgenomic transcript. Finally, CSE 4, which is located just upstream of the poly(A) sequence in the 3′ untranslated region of the alphavirus genome, is understood to function as a core promoter for initiation of (−) strand synthesis (José et al., 2009, Future Microbiol. 4:837-856). CSE 4 and the poly(A) tail of the alphavirus are understood to function together for efficient (−) strand synthesis (Hardy & Rice, 2005, J. Virol. 79:4630-4639). In addition to alphavirus proteins, also host cell factors, presumably proteins, may bind to conserved sequence elements. The 5′ replication recognition sequence of the alphavirus genome is not only involved in translation initiation, but also comprises two conserved sequence elements involved in synthesis of viral RNA, CSE 1 and CSE 2. For the function of CSE 1 and 2, the secondary structure is believed to be more important than the linear sequence (Strauss & Strauss, 1994, Microbiol. Rev. 58:491-562).

Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase (typically as poly-protein nsP1234), and the other nucleic acid molecule is capable of being replicated by said replicase in trans (hence the designation trans-replication system). trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Given the success of modified mRNA vaccines, it appears attractive to modify saRNA in order to reduce the innate immune response. However, modified nucleotides have been observed to inhibit the replication and translation of saRNA (Erasmus et al., 2020, Sci Transl Med 12: eabc9396). To date, there is no published investigation why RNA modification is incompatible with saRNA function. Thus, there remains a need in the art for saRNA molecules that possess sufficient replication and/or translation function despite containing modified nucleotides. The present invention fulfils such need.

### SUMMARY

The present invention generally relates to improving the ability of self-replicating RNA molecules, also called replicons or replicable RNA (rRNA or saRNA), that contain nucleotides other than uracil, adenosine, cytosine and guanine, to replicate and/or be translated to express an encoded protein. Thus, the invention relates to methods for identifying sequence changes that restore or improve the decreased ability of replicable RNA molecules that contain modified nucleotides to replicate and/or be translated. Additionally, the invention relates to replicable RNA molecules comprising these sequence changes, and the use of such replicable RNA molecules in methods for expressing a protein in a cell, or for raising an immune response, preferably a cytotoxic immune response, against proteins encoded by the replicable RNA molecules, as well as for treating or preventing diseases or disorders in which such an immune response leads to/results in such treatment or prevention of the disease or disorder.

The present invention is based, in part, on the hypothesis that RNA secondary structures within the 5′- or 3′-conserved sequence elements (CSE) change their shape or stability upon modification of the nucleotides. RNA replication depends upon an interaction of the RNA template with replicase. Thus, improper interaction of RNA with the replicase will profoundly affect RNA-dependent RNA-transcription. Since RNA structures depend on the nucleotide sequence, the inventors proposed that modified RNA can adapt a structure interacting properly with replicase upon changes in the sequence by replacing the canonical nucleotides of A, C, G and U with modified nucleotides, such as N1-methyl-pseudouracil. The present inventors showed, as demonstrated by the experimental results disclosed herein, that an in vitro evolution method allowed for the identification of sequence changes allowing for restored or improved function of the rRNA.

In one aspect, the invention relates to a method for identifying a sequence change in a replicable RNA (rRNA) comprising a modified nucleotide (modified rRNA) that at least partially restores or increases a function of the modified rRNA, the method comprising (a) transfecting cells expressing an RNA-dependent RNA polymerase (replicase) with a modified rRNA encoding a gene of interest; and (b) identifying a sequence change within the rRNA molecules encoding the gene of interest present in the transfected cells expressing the gene of interest. The rRNA can be a single-stranded RNA molecule that can be translated (+-stranded) and may or may not encode an RNA-dependent RNA polymerase (replicase) but comprises nucleotide sequences which enable the molecule to be replicated in trans by a separately provided replicase or in cis by a replicase encoded by the same rRNA. An rRNA molecule also can be called a trans-replicon (TR) or replicon. In addition, the rRNA may comprise wildtype or codon-optimized sequences encoding a gene sequence, e.g., an antigen, and the rRNA may contain one or more structural elements optimized for maximal efficacy of the rRNA with respect to stability and translational efficiency (5′ cap, 5′ UTR, 3′ UTR, poly(A)-tail). In an embodiment, the sequence change is present in the 5′ regulatory region of a replicon.

In an embodiment, the restoration or improvement of the function of the rRNA or nucleotide-modified rRNA may be determined by the restoration or improvement in the level of expression of the gene of interest, e.g., after a fixed period of time after transfection, such as after 1, 2, 3, 4, 5, 6, 12, 18, 24 hours or 1, 2, 3, 4, or 5 days after transfection. In an embodiment, the restoration or improvement of the function of the rRNA may be determined by the restoration or improvement in the ability of the rRNA to be replicated, as measured, e.g., by total number of copies of the rRNA in the transfected cells after a fixed period of time such as 1, 2, 3, 4, 5, 6, 12, 18, 24 hours or 1, 2, 3, 4, or 5 days after transfection.

In an embodiment, the sequence change can be determined relative to the sequence of the modified rRNA transfected in step (a). The sequence change also may be determined relative to a reference sequence. In one embodiment, step (a) of the method may further comprise, after transfecting, isolating rRNA molecules encoding the gene of interest from the transfected cells expressing the gene of interest to provide isolated rRNA molecules. In one embodiment, step (a) may further comprises a step of transfecting cells expressing the replicase with a subsequent generation of rRNA molecules encoding the gene of interest and comprising the modified nucleotide, which subsequent generation can be provided by replicating the isolated rRNA molecules.

In an embodiment, the method may further comprise repeating the steps of isolating rRNA molecules encoding the gene of interest from the transfected cells expressing the gene of interest providing isolated rRNA molecules and transfecting cells expressing the replicase with a subsequent generation of rRNA molecules encoding the gene of interest and comprising the modified nucleotide, which subsequent generation can be provided by replicating the isolated rRNA molecules. The steps may be repeated a number of (n) times, in which n may be an integer of at least 1. The steps can be repeated 1, 2, 3, 4, 5, 6, 7, 8, 9, 10, 11, 12, 13, 14, 15, 16, 17, 18, 19, 20 or more times. In an embodiment, replicating the isolated rRNA molecules comprises (i) reverse-transcribing the isolated rRNA molecules encoding the gene of interest to form DNA molecules that are capable of being in vitro transcribed; and (ii) in vitro transcribing the DNA molecules in the presence of the same modified nucleotide to produce a subsequent generation of modified rRNA molecules comprising the modified nucleotide and encoding the gene of interest. The reverse transcribing may be accomplished by a method comprising one-step reverse transcription PCR (RT-PCR).

In an embodiment, the method may further comprise the steps (c) incorporating at least one identified sequence change into the modified rRNA of step (a); (d) transfecting cells expressing the replicase with the modified rRNA produced in step (c); and (e) identifying a sequence change within the rRNA molecules isolated from the transfected cells expressing the gene of interest produced in step (d). In an embodiment, the cells used in the method are those without an interferon response.

In some embodiments the modified nucleotide(s) in the replicable RNA (rRNA) are not, or are other than, a 5′cap structure. In an embodiment, the number of modified nucleotides in the modified rRNA useful in the method can be at least 10%, 20%, 30%, 40%, 50%, 60%, 70%, 80%, 90% or 100% of the total number of nucleotides in the rRNA. Preferably, the number of modified nucleotides in the modified rRNA useful in the method can be at least 30% of the total number of nucleotides in the rRNA. More preferably, the number of modified nucleotides in the modified rRNA useful in the method can be at least 50% of the total number of nucleotides in the rRNA. The modified nucleotide can be a modified uridine residue, a modified adenine residue, a modified guanine residue, a modified cytosine residue, or any combination of two or more of the foregoing. In an embodiment, the modified nucleotide is a modified uridine, such as pseudouridine or N1-methyl-pseudouridine. In an embodiment, the number of modified uridine residues in the in vitro transcribed modified rRNA can be at least 10%, 20%, 30%, 40%, 50%, 60%, 70%, 80%, 90% or 100% of the total number of uridine residues. The number of modified uridine residues (e.g., pseudouridine or N1-methyl-pseudouridine residues) in the in vitro transcribed modified rRNA can be at least 30% of the total number of uridine residues. Preferably, the number of modified uridine residues (e.g., pseudouridine or N1-methyl-pseudouridine residues) in the in vitro transcribed modified rRNA can be at least 50% of the total number of uridine residues. The number of modified uridine residues (e.g., pseudouridine or N1-methyl-pseudouridine residues) in the in vitro transcribed modified rRNA can be about 100% (i.e., substantially all) of the total number of uridine residues. In an embodiment, the number of N1-methyl-pseudouridine residues in the in vitro transcribed modified rRNA can be at least 10%, 20%, 30%, 40%, 50%, 60%, 70%, 80%, 90% or 100% of the total number of uridine residues. Preferably, the number of N1-methyl-pseudouridine residues in the in vitro transcribed modified rRNA can be at least 30% of the total number of uridine residues. More preferably, the number of N1-methyl-pseudouridine residues in the in vitro transcribed modified rRNA can be at least 50% of the total number of uridine residues.

In an embodiment, the rRNA useful in the method essentially is the genome of a single-, positive-stranded RNA virus, e.g., alphavirus, picornavirus, flavivirus, which optionally does not encode a functional structural protein of the virus.

In an embodiment, the replicase useful in the method may be derived from a self-replicating RNA virus, such as an alphavirus, e.g., Semliki Forest virus (SFV), Venezuelan equine encephalitis virus (VEEV), Sindbis virus, Eastern equine encephalitis virus (EEEV), Western equine encephalitis virus, or Chikungunya virus. In an embodiment, the alphavirus can be SFV or VEEV or EEEV or Sindbis virus.

In an embodiment, the rRNA useful in the method comprises a 5′ regulatory region that has no start codons. In an embodiment, the replicase and the regulatory sequences in the rRNA useful in the method required by the replicase for replication are derived from the same alphavirus or from different alphaviruses.

In an embodiment, where the rRNA also comprises an open reading frame encoding a replicase, translation of the replicase open reading frame is uncoupled from a 5′-terminal cap by placing translation of the replicase open reading frame under the translational control of an internal ribosome entry site (IRES). In an embodiment, the rRNA may be uncapped. In an embodiment, the rRNA may have open reading frames for expression of additional genes upstream to the IRES.

In an embodiment, the encoded gene of interest can be an antigen or a reporter gene, such as a cell surface expressed protein or luciferase or GFP.

In an embodiment, the expression of the replicase in the cells useful in the method can be by transient expression or by constitutive expression. In an embodiment, the cells are transfected with a nucleic acid molecule encoding the replicase prior to, concurrently with, or after transfection of the rRNA (not necessarily encoding the replicase). In an embodiment, the nucleic acid molecule encoding the replicase is an RNA molecule, such as an mRNA molecule. In an embodiment, step (a) of the method comprises transfecting cells with a self-amplifying RNA encoding a gene of interest, which self-replicating RNA comprises at least one modified nucleotide.

In an embodiment, the function of the rRNA that is partially restored or increased can be the ability to be replicated by a replicase and/or can be the ability to be translated; for example, as directed by the 5′ regulatory region. In an embodiment, the function can be restored by at least 10% or can be increased by at least 10%.

In an embodiment, the step of identifying the sequence change can comprise sequencing at least a portion of the rRNA molecules. In an embodiment, the portion of the rRNA that is sequenced can be the 5′ regulatory region and/or the portion of the rRNA that is sequenced is the 3′ regulatory region. In an embodiment, the sequence change can be determined by analyzing sequence-specific read frequencies generated by sequencing such as next-generation sequencing.

In an embodiment, the method can be carried out to increase the replication efficiency of a self-amplifying RNA comprising at least one modified nucleotide.

In an embodiment, the method further comprises modifying the nucleotide sequence of an rRNA or self-replicating RNA comprising the same modified nucleotide by incorporating at least one identified sequence change that partially restores or increases the function of the rRNA or self-replicating RNA comprising the modified nucleotide.

In an aspect, the present invention is directed to a replicable RNA (rRNA) molecule obtained by the method of the invention described above, which molecule comprises one or more modified nucleotides. In an embodiment, the obtained rRNA molecule comprises a mutated 5′ regulatory region of a self-replicating RNA virus. In an embodiment, the mutated 5′ regulatory region comprises one or more point mutations. In an particular embodiment, the rRNA molecule obtained by the method of the invention comprising one or more modified nucleotides is able to be replicated/be transcribed by a replicase at an increased level compared to the parental rRNA molecule not subject to the method. In an embodiment, the rRNA molecule comprises at least 30%, 50%, 75% modified nucleotides out of the total nucleotides of the rRNA molecule. Accordingly, in some embodiments, the invention provides a replicable RNA (rRNA) molecule which is obtainable by the method of the invention as described herein, wherein at least 50% (preferably at least 75%, most preferably about 100%) of the total number of uridine residues in the rRNA molecule are modified uridine residues (e.g., pseudouridine or N1-methyl-pseudouridine residues), and wherein the rRNA molecule is capable of being replicated/transcribed by a replicase at a similar or increased level compared to the parental rRNA molecule not subject to the method. In some embodiments, the rRNA molecule may be capable of being replicated/transcribed by a replicase at a similar or increased level compared to a corresponding rRNA molecule comprising a wild-type 5′ regulatory region of a rRNA molecule, optionally compared to a corresponding rRNA molecule having a 5′ regulatory region comprising SEQ ID NO: 1 or SEQ ID NO: 2 (wild type or modified version of the 5′ regulatory region of VEEV Trinidad donkey strain (Accession No. L01442)). The replicase may be an alphaviral replicase. In some embodiments, the replicase comprises non-structural protein from Semliki Forest virus (SFV), Sindbis virus, Venezuelan equine encephalitis virus (VEEV), Eastern equine encephalitis virus (EEEV), or chikungunya virus (CHIKV), preferably SFV, VEEV or EEV, more preferably SFV.

In an aspect of the invention, the modified, mutated rRNA molecule comprises a modified 5′ regulatory region of a self-replicating RNA virus, which modified regulatory region can comprise a point mutation at one or more of positions 67, 244, 245, 246, 248 of the 5′ regulatory region (SEQ ID NO: 2) or to positions in the 5′ regulatory region of an alphavirus corresponding to those positions in SEQ ID NO: 2.

In an embodiment, the rRNA molecule can comprise at least one modified nucleotide or nucleotide.

SEQ ID NO: 2 is the 5′ regulatory sequence of VEEV Trinidad donkey strain (Accession No. L01442) which is, inter alia, modified at the viral 5′ end by the addition of a G residue compared to the 5′ end of the wild type sequence (SEQ ID NO: 1). This modification in the DNA template of the RNA allows for efficient in vitro transcription using the T7 polymerase.

In an embodiment, the rRNA molecule can further comprise in the 5′ regulatory region a point mutation at position 4 of the 5′ regulatory region (SEQ ID NO: 2) or at a position in the 5′ regulatory region of an alphavirus corresponding to that position in SEQ ID NO: 2, preferably a substitution of the guanine residue with an adenosine residue (G4A).

In an embodiment, the rRNA molecule does not comprise in the 5′ regulatory region a point mutation at position 4 of the 5′ regulatory region (SEQ ID NO: 2) or at a position in the 5′ regulatory region of an alphavirus corresponding to that position in SEQ ID NO: 2, preferably not a substitution of the guanine residue with an adenosine residue (G4A).

The self-replicating RNA virus can be an alphavirus.

SEQ ID NO: 1 is the wild-type 5′ regulatory region of VEEV Trinidad donkey strain (Accession No. L01442), which does not contain the 5′-most G residue. Thus, positions 4, 67, 244, 245, 246 and 248 in SEQ ID NO: 2 correspond to positions 3, 66, 243, 244, 245 and 247 in SEQ ID NO: 1, respectively, and the identity of the nucleotide at positions 4, 67, 244, 245, 246 and 248 in SEQ ID NO: 2 is the same as the identity of the nucleotide at positions 3, 66, 243, 244, 245 and 247 in SEQ ID NO: 1, respectively.

In various embodiments, the point mutation may be at position 67, at positions 67 and 244, at positions 67 and 246, at positions 67 and 248, at positions 67, 245 and 248, at positions 4 and 67, at positions 4, 67 and 244, at positions 4, 67 and 246, at positions 4, 67 and 248, at positions 4, 67, 245 and 248 of SEQ ID NO: 2, or any combination thereof, or at positions in alphaviruses corresponding to such positions in SEQ ID NO: 2.

In various embodiments, the point mutation may be at one or more of the following positions or combination of positions of SEQ ID NO: 2 or at corresponding positions in the 5′ regulatory region of an alphavirus:
67; 244; 245; 246; 248; 67 and 244; 67 and 245; 67 and 246; 67 and 248; 244 and 245; 244 and 246; 244 and 248; 245 and 246; 245 and 248; 246 and 248; 67, 244 and 245; 67, 244 and 246; 67, 244 and 248; 67, 245 and 246; 67, 245 and 248; 67, 246 and 248; 244, 245 and 246; 244, 245 and 248; 244, 246 and 248; 245, 246 and 248; 67, 244, 245 and 246; 67, 244, 245 and 248; 67, 244, 246 and 248; 67, 245, 246 and 248; 244, 245, 246 and 248; 67, 244, 245, 246 and 248.

In an embodiment, the modified regulatory region of the rRNA molecule comprises a point mutation at position 67 or to a position in the 5′ regulatory region of an alphavirus corresponding to this position in SEQ ID NO: 2 and at one or more of positions 244, 245, 246, 248 of the 5′ regulatory region (SEQ ID NO: 2) or to positions in the 5′ regulatory region of an alphavirus corresponding to those positions in SEQ ID NO: 2.

In each of the foregoing positions or combination of positions, a point mutation further may be at position 4 of SEQ ID NO: 2 or at the corresponding position in the 5′ regulatory region of an alphavirus.

In an embodiment, the point mutation at the various positions may be G4A, A67C, G244A, C245A, G246A, or C248A.

In an embodiment, the self-replicating RNA virus can be SFV or VEEV or EEEV or Sindbis virus.

In an embodiment, the corresponding position in another alphavirus can be the same position relative to the first nucleotide of the 5′ regulatory region and/or the same relative position found in one of the stem/loop structures found in the other alphaviral 5′ regulatory region.

In an embodiment, the rRNA molecule can further comprise one or more coding regions, for example one or more coding regions comprising a sequence encoding a gene of interest. The encoded gene of interest, for example, can be an antigen or a therapeutic protein or a nucleic acid or a reporter gene. In an embodiment, the antigen is a tumor, viral, bacterial, or fungal antigen, or an allergen.

In an embodiment, the rRNA molecule can encode an RNA-dependent RNA polymerase (replicase), for example, an alphaviral replicase. In an embodiment, the rRNA molecule can comprise a 5′ regulatory region and a coding sequence for a replicase, wherein the 5′ regulatory region and the encoded replicase are derived from the same self-replicating RNA virus, for example, from the same alphavirus. In an embodiment, the rRNA molecule can comprise a 5′ regulatory region and a coding sequence for a replicase, wherein the 5′ regulatory region and the encoded replicase are derived from different self-replicating RNA viruses, for example, different alphaviruses.

In an embodiment, the rRNA molecule may be a self-amplifying RNA, i.e., one which encodes a replicase. In an embodiment, the rRNA molecule may be an in vitro transcribed rRNA, that is produced in an in vitro transcription reaction, i.e., not in a cell.

In an embodiment, where the rRNA also comprises an open reading frame encoding a replicase, translation of the replicase open reading frame can be uncoupled from a 5′-terminal cap by placing translation of the replicase open reading frame under the translational control of an internal ribosome entry site (IRES). In an embodiment, the rRNA may be uncapped. In an embodiment, the rRNA may have open reading frames for expression of additional genes upstream to the IRES.

In an embodiment, the rRNA molecule comprises at least one modified nucleotide, that is a nucleotide that is not an adenosine, guanine, cytosine or uracil, e.g., as described above. As used herein, the modified nucleotide can be modified in the backbone linkage, sugar moiety or base, and includes a modified nucleoside. In an embodiment, the modified nucleotide may be N1-methyl-pseudouridine. In an embodiment, all of the uridine residues of the rRNA are N1-methyl-pseudouridine residues.

In an embodiment, the rRNA can be a stabilized rRNA. In an embodiment, the rRNA can comprise a modified nucleoside in place of at least one uridine. In an embodiment, the rRNA can comprise a modified nucleoside in place of each uridine. In one embodiment, the modified nucleoside can be independently selected from pseudouridine (ψ), N1-methyl-pseudouridine (m1ψ or 1mY), and 5-methyl-uridine (m5U).

In an aspect, the present invention is directed to an RNA molecule comprising the sequence depicted in SEQ ID NO: 2 in which one or more of positions 4, 67, 244, 245, 246 and 248 have a point mutation. In various embodiments, the point mutation may be at positions 67; 244; 245; 246; 248; 67 and 244; 67 and 245; 67 and 246; 67 and 248; 244 and 245; 244 and 246; 244 and 248; 245 and 246; 245 and 248; 246 and 248; 67, 244 and 245; 67, 244 and 246; 67, 244 and 248; 67, 245 and 246; 67, 245 and 248; 67, 246 and 248; 244, 245 and 246; 244, 245 and 248; 244, 246 and 248; 245, 246 and 248; 67, 244, 245 and 246; 67, 244, 245 and 248; 67, 244, 246 and 248; 67, 245, 246 and 248; 244, 245, 246 and 248; 67, 244, 245, 246 and 248. In an embodiment, the RNA molecule comprises the sequence depicted in SEQ ID NO: 2 in which position 67 and one or more of positions 4, 244, 245, 246 and 248 have a point mutation. In each of the foregoing positions or combination of positions, a further point mutation may be at position 4. In an embodiment, the point mutations can be G4A, A67C, G244A, C245A, G246A, and/or C248A.

In an aspect, the present invention relates to a DNA molecule that encodes the rRNA molecule comprising one or more of the point mutations at the positions disclosed herein in SEQ ID NO: 2. In an embodiment, the rRNA molecule or the DNA molecule may be linear or circular.

In an embodiment, the rRNA or DNA molecule may be formulated with a reagent capable of forming particles with the rRNA or DNA molecules, for example, the reagent can be a lipid or polyalkyleneimine. In various embodiments, the lipid can comprise a cationic headgroup, and/or the lipid can be a pH responsive lipid, and/or the lipid can be a PEGylated-lipid. In an embodiment, the reagent can be conjugated to polysarcosine. In an embodiment, the particles formed from the rRNA or DNA molecules and the reagent can be polymer-based polyplexes (PLX) or lipid nanoparticles (LNP), wherein the LNP is preferably a lipoplex (LPX) or a liposome. In an embodiment, the particle can further comprise at least one phosphatidylserine. In an embodiment, the particles can be nanoparticles, in which (i) the number of positive charges in the nanoparticles does not exceed the number of negative charges in the nanoparticles and/or (ii) the nanoparticles have a neutral or net negative charge and/or (iii) the charge ratio of positive charges to negative charges in the nanoparticles is 1.4:1 or less and/or (iv) the zeta potential of the nanoparticles is 0 or less. In an embodiment, the charge ratio of positive charges to negative charges in the nanoparticles can be between 1.4:1 and 1:8, preferably between 1.2:1 and 1:4.

In one embodiment, the rRNA or DNA can be formulated or is to be formulated as a liquid, a solid, or a combination thereof. In one embodiment, the rRNA or DNA can be formulated or is to be formulated for injection. In one embodiment, the rRNA or DNA can be formulated or is to be formulated for intramuscular administration. In one embodiment, the rRNA or DNA can be formulated or is to be formulated as particles. In one embodiment, the particles are lipid nanoparticles (LNP) or lipoplex (LPX) particles. In one embodiment, the LNP particles comprise ((4-hydroxybutyl)azanediyl)bis(hexane-6,1-diyl)bis(2-hexyldecanoate), 2-[(polyethylene glycol)-2000]-N,N-ditetradecylacetamide, 1,2-Distearoyl-sn-glycero-3-phosphocholine, and cholesterol.

In one embodiment, the rRNA lipoplex particles can be obtained by mixing the rRNA with liposomes. In one embodiment, the rRNA lipoplex particles can be obtained by mixing the rRNA with lipids.

In one embodiment, the rRNA can be formulated or is to be formulated as colloid. In one embodiment, the rRNA can be formulated or is to be formulated as particles, forming the dispersed phase of a colloid. In one embodiment, 50% or more, 75% or more, or 85% or more of the rRNA are present in the dispersed phase. In one embodiment, the rRNA can be formulated or is to be formulated as particles comprising rRNA and lipids. In one embodiment, the particles can be formed by exposing rRNA, dissolved in an aqueous phase, with lipids, dissolved in an organic phase. In one embodiment, the organic phase can comprise ethanol. In one embodiment, the particles can be formed by exposing rRNA, dissolved in an aqueous phase, with lipids, dispersed in an aqueous phase. In one embodiment, the lipids dispersed in an aqueous phase form liposomes.

In an aspect, the present invention relates to a pharmaceutical composition comprising the rRNA molecule comprising the point mutations described herein or the DNA molecule encoding such rRNA molecule and a pharmaceutically acceptable carrier or excipient.

In an aspect, the present invention relates to a method of expressing a gene product of interest in a cell, said method comprising providing to the cell a rRNA, which rRNA comprises a modified 5′ regulatory region of a self-replicating RNA virus, preferably an alphavirus, which modified regulatory region comprises a point mutation at one or more of positions 67, 244, 245, 246, 248 of SEQ ID NO: 2, preferably at position 67 and at one or more of positions 244, 245, 246, 248 of SEQ ID NO: 2, and an open reading frame encoding for the gene product of interest.

In an aspect, the present invention relates to the rRNA comprising the point mutations described herein or the encoding DNA molecule or the pharmaceutical composition comprising the rRNA or DNA molecule may be used in therapy. In an aspect, the present invention relates to a method for raising an immune response in a subject comprising administering an rRNA molecule encoding an antigen to the subject, and which rRNA molecule comprises a modified 5′ regulatory region of a self-replicating RNA virus, preferably an alphavirus, which modified regulatory region comprises a point mutation at one or more of positions 67, 244, 245, 246, 248 of SEQ ID NO: 2, preferably at position 67 and at one or more of positions 244, 245, 246, 248 of SEQ ID NO: 2.

In an aspect, the present invention relates to a method for treating cancer in a subject comprising administering an rRNA molecule encoding a tumor antigen to the subject, and which rRNA comprises a modified 5′ regulatory region of a self-replicating RNA virus, preferably an alphavirus, which modified regulatory region comprises a point mutation at one or more of positions 67, 244, 245, 246, 248 of SEQ ID NO: 2, preferably at position 67 and at one or more of positions 244, 245, 246, 248 of SEQ ID NO: 2.

In an aspect, the present invention relates to a method for providing gene function to a subject lacking such gene function comprising administering an rRNA molecule encoding a gene product (therapeutic protein) to the subject to provide the gene function, and which rRNA molecule comprises a modified 5′ regulatory region of self-replicating RNA virus, preferably an alphavirus, which modified regulatory region comprises a point mutation at one or more of positions 67, 244, 245, 246, 248 of SEQ ID NO: 2, preferably at position 67 and at one or more of positions 244, 245, 246, 248 of SEQ ID NO: 2.

In an embodiment, the point mutation further may be at position 4 of SEQ ID NO: 2. In an embodiment, the rRNA molecule useful for expression of a gene product or in therapy comprises at least one modified nucleotide, for example, N1-methyl-pseudouridine. Such rRNA may be a self-amplifying RNA molecule in that it further comprises sequences encoding a replicase.

Any of the foregoing characteristics of rRNA molecules may equally apply to the rRNA used in the methods of the invention used to identify sequence changes for restoring or improving function of the rRNA, as well as in the rRNA molecules which have such identified sequence changes incorporated into its sequence.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise.

The term “about” means approximately or nearly, and in the context of a numerical value or range set forth herein preferably means +/−10% of the numerical value or range recited or claimed.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless expressly specified otherwise, the term “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e., for the purpose of this embodiment “comprising” is to be understood as having the meaning of “consisting of”.

Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present invention was not entitled to antedate such disclosure.

Terms such as “reduce” or “inhibit” as used herein means the ability to cause an overall decrease, preferably of 5% or greater, 10% or greater, 20% or greater, more preferably of 50% or greater, and most preferably 75% or greater, in the level. The term “inhibit” or similar phrases includes a complete or essentially complete inhibition, i.e., a reduction to zero or essentially to zero.

Terms such as “increase” or “enhance” preferably relate to an increase or enhancement by about at least 10%, preferably at least 20%, preferably at least 30%, more preferably at least 40%, more preferably at least 50%, even more preferably at least 80%, and most preferably at least 100%.

The term “net charge” refers to the charge on a whole object, such as a compound or particle.

An ion having an overall net positive charge is a cation, while an ion having an overall net negative charge is an anion. Thus, according to the invention, an anion is an ion with more electrons than protons, giving it a net negative charge; and a cation is an ion with fewer electrons than protons, giving it a net positive charge.

Terms as “charged”, “net charge”, “negatively charged” or “positively charged”, with reference to a given compound or particle, refer to the electric net charge of the given compound or particle when dissolved or suspended in water at pH 7.0.

The term “nucleic acid” according to the invention also comprises a chemical derivatization of a nucleic acid on a nucleotide base, on the sugar or on the phosphate, and nucleic acids containing non-natural nucleotides and nucleotide analogs. In some embodiments, the nucleic acid is a deoxyribonucleic acid (DNA) or a ribonucleic acid (RNA). In general, a nucleic acid molecule or a nucleic acid sequence refers to a nucleic acid which is preferably deoxyribonucleic acid (DNA) or ribonucleic acid (RNA). According to the invention, nucleic acids comprise genomic DNA, cDNA, mRNA, viral RNA, recombinantly prepared and chemically synthesized molecules. According to the invention, a nucleic acid may be in the form of a single-stranded or double-stranded and linear or covalently closed circular molecule.

According to the invention “nucleic acid sequence” refers to the sequence of nucleotides in a nucleic acid, e.g.; a ribonucleic acid (RNA) or a deoxyribonucleic acid (DNA). The term may refer to an entire nucleic acid molecule (such as to the single strand of an entire nucleic acid molecule) or to a part (e.g. a fragment) thereof.

According to the present invention, the term “RNA” or “RNA molecule” relates to a molecule which comprises ribonucleotide residues and which is preferably entirely or substantially composed of ribonucleotide residues. The term “ribonucleotide” relates to a nucleotide with a hydroxyl group at the 2′-position of a β-D-ribofuranosyl group. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of an RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally occurring RNAs.

According to the invention, RNA may be single-stranded or double-stranded. In some embodiments of the present invention, single-stranded RNA is preferred. The term “single-stranded RNA” generally refers to an RNA molecule to which no complementary nucleic acid molecule (typically no complementary RNA molecule) is associated. Single-stranded RNA may contain self-complementary sequences that allow parts of the RNA to fold back and to form secondary structure motifs including without limitation base pairs, stems, stem loops and bulges. Single-stranded RNA can exist as minus strand [(−) strand] or as plus strand [(+) strand]. The (+) strand is the strand that comprises or encodes genetic information. The genetic information may be for example a polynucleotide sequence encoding a protein. When the (+) strand RNA encodes a protein, the (+) strand may serve directly as template for translation (protein synthesis). The (−) strand is the complement of the (+) strand. In the case of double-stranded RNA, (+) strand and (−) strand are two separate RNA molecules, and both these RNA molecules associate with each other to form a double-stranded RNA (“duplex RNA”).

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period.

The term “translation efficiency” relates to the amount of translation product provided by an RNA molecule within a particular period of time.

“Fragment”, with reference to a nucleic acid sequence, relates to a part of a nucleic acid sequence, i.e.; a sequence which represents the nucleic acid sequence shortened at the 5′- and/or 3′-end(s). Preferably, a fragment of a nucleic acid sequence comprises at least 80%, preferably at least 90%, 95%, 96%, 97%, 98%, or 99% of the nucleotide residues from said nucleic acid sequence. In the present invention those fragments of RNA molecules are preferred which retain RNA stability and/or translational efficiency.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises e.g. at least 1%, at least 2%, at least 3%, at least 4%, at least 5%, at least 10%, at least 20%, at least 30%, at least 40%, at least 50%, at least 60%, at least 70%, at least 80%, at least 90% of the amino acid residues from an amino acid sequence.

The term “variant” with respect to, for example, nucleic acid and amino acid sequences, according to the invention includes any variants, in particular mutants, viral strain variants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. With respect to nucleic acid molecules, the term “variant” includes degenerate nucleic acid sequences, wherein a degenerate nucleic acid according to the invention is a nucleic acid that differs from a reference nucleic acid in codon sequence due to the degeneracy of the genetic code. A species homolog is a nucleic acid or amino acid sequence with a different species of origin from that of a given nucleic acid or amino acid sequence. A virus homolog is a nucleic acid or amino acid sequence with a different virus of origin from that of a given nucleic acid or amino acid sequence.

According to the invention, nucleic acid variants include single or multiple nucleotide deletions, additions, mutations, substitutions and/or insertions in comparison with the reference nucleic acid. Deletions include removal of one or more nucleotides from the reference nucleic acid. Addition variants comprise 5′- and/or 3′-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations include abasic sites, crosslinked sites, and chemically altered or modified bases. Insertions include the addition of at least one nucleotide into the reference nucleic acid.

According to the invention, “nucleotide change” can refer to single or multiple nucleotide deletions, additions, mutations, substitutions and/or insertions in comparison with the reference nucleic acid. In some embodiments, a “nucleotide change” is selected from the group consisting of a deletion of a single nucleotide, the addition of a single nucleotide, the mutation of a single nucleotide, the substitution of a single nucleotide and/or the insertion of a single nucleotide, in comparison with the reference nucleic acid. According to the invention, a nucleic acid variant can comprise one or more nucleotide changes in comparison with the reference nucleic acid.

Variants of specific nucleic acid sequences preferably have at least one functional property of said specific sequences and preferably are functionally equivalent to said specific sequences, e.g., nucleic acid sequences exhibiting properties identical or similar to those of the specific nucleic acid sequences.

As described below, some embodiments of the present invention are characterized, inter alia, by nucleic acid sequences that are homologous to other nucleic acid sequences. These homologous sequences are variants of other nucleic acid sequences.

Preferably the degree of identity between a given nucleic acid sequence and a nucleic acid sequence which is a variant of said given nucleic acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence.

“Sequence similarity” indicates the percentage of amino acids that either are identical or that represent conservative amino acid substitutions. “Sequence identity” between two polypeptide or nucleic acid sequences indicates the percentage of amino acids or nucleotides that are identical between the sequences.

The term “% identical” is intended to refer, in particular, to a percentage of nucleotides which are identical in an optimal alignment between two sequences to be compared, with said percentage being purely statistical, and the differences between the two sequences may be randomly distributed over the entire length of the sequence and the sequence to be compared may comprise additions or deletions in comparison with the reference sequence, in order to obtain optimal alignment between two sequences. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2:482, with the aid of the local homology algorithm by Needleman and Wunsch, 1970, J. Mol. Biol. 48:443, and with the aid of the similarity search algorithm by Pearson and Lipman, 1988, Proc. Natl Acad. Sci. USA 85:2444 or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions in which the sequences to be compared correspond, dividing this number by the number of positions compared and multiplying this result by 100.

For example, the BLAST program “BLAST 2 sequences” which is available on the website http://www.ncbi.nlm.nih.gov/blast/bl2seq/wblast2.cgi may be used.

A nucleic acid is “capable of hybridizing” or “hybridizes” to another nucleic acid if the two sequences are complementary with one another. A nucleic acid is “complementary” to another nucleic acid if the two sequences are capable of forming a stable duplex with one another. According to the invention, hybridization is preferably carried out under conditions which allow specific hybridization between polynucleotides (stringent conditions). Stringent conditions are described, for example, in Molecular Cloning: A Laboratory Manual, J. Sambrook et al., Editors, 2nd Edition, Cold Spring Harbor Laboratory press, Cold Spring Harbor, New York, 1989 or Current Protocols in Molecular Biology, F. M. Ausubel et al., Editors, John Wiley & Sons, Inc., New York and refer, for example, to hybridization at 65° C. in hybridization buffer (3.5×SSC, 0.02% Ficoll, 0.02% polyvinylpyrrolidone, 0.02% bovine serum albumin, 2.5 mM NaH2PO4 (pH 7), 0.5% SDS, 2 mM EDTA). SSC is 0.15 M sodium chloride/0.15 M sodium citrate, pH 7. After hybridization, the membrane to which the DNA has been transferred is washed, for example, in 2×SSC at room temperature and then in 0.1-0.5×SSC/0.1×SDS at temperatures of up to 68° C.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or “fully complementary” means that all the contiguous residues of a nucleic acid sequence will hydrogen bond with the same number of contiguous residues in a second nucleic acid sequence. Preferably, the degree of complementarity according to the invention is at least 70%, preferably at least 75%, preferably at least 80%, more preferably at least 85%, even more preferably at least 90% or most preferably at least 95%, 96%, 97%, 98% or 99%. Most preferably, the degree of complementarity according to the invention is 100%.

The term “derivative” comprises any chemical derivatization of a nucleic acid on a nucleotide base, on the sugar or on the phosphate. The term “derivative” also comprises nucleic acids which contain nucleotides and nucleotide analogs not occurring naturally. Preferably, a derivatization of a nucleic acid increases its stability.

A “nucleic acid sequence which is derived from a nucleic acid sequence” refers to a nucleic acid which is a variant of the nucleic acid from which it is derived. Preferably, a sequence which is a variant with respect to a specific sequence, when it replaces the specific sequence in an RNA molecule retains RNA stability and/or translational efficiency.

“nt” is an abbreviation for nucleotide; or for nucleotides, preferably consecutive nucleotides in a nucleic acid molecule.

According to the invention, the term “codon” refers to a base triplet in a coding nucleic acid that specifies which amino acid will be added next during protein synthesis at the ribosome.

The terms “transcription” and “transcribing” relate to a process during which a nucleic acid molecule with a particular nucleic acid sequence (the “nucleic acid template”) is read by an RNA polymerase so that the RNA polymerase produces a single-stranded RNA molecule. During transcription, the genetic information in a nucleic acid template is transcribed. The nucleic acid template may be DNA; however, e.g.; in the case of transcription from an alphaviral nucleic acid template, the template is typically RNA. Subsequently, the transcribed RNA may be translated into protein. According to the present invention, the term “transcription” comprises “in vitro transcription”, wherein the term “in vitro transcription” relates to a process wherein RNA, in particular mRNA, is in vitro synthesized in a cell-free system. Preferably, cloning vectors are applied for the generation of transcripts. These cloning vectors are generally designated as transcription vectors and are according to the present invention encompassed by the term “vector”. The cloning vectors are preferably plasmids. According to the present invention, RNA preferably is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

The single-stranded nucleic acid molecule produced during transcription typically has a nucleic acid sequence that is the complementary sequence of the template.

According to the invention, the terms “template” or “nucleic acid template” or “template nucleic acid” generally refer to a nucleic acid sequence that may be replicated or transcribed.

“Nucleic acid sequence transcribed from a nucleic acid sequence” and similar terms refer to a nucleic acid sequence, where appropriate as part of a complete RNA molecule, which is a transcription product of a template nucleic acid sequence. Typically, the transcribed nucleic acid sequence is a single-stranded RNA molecule.

“3′ end of a nucleic acid” refers according to the invention to that end which has a free hydroxy group. In a diagrammatic representation of double-stranded nucleic acids, in particular DNA, the 3′ end is always on the right-hand side. “5′ end of a nucleic acid” refers according to the invention to that end which has a free phosphate group. In a diagrammatic representation of double-strand nucleic acids, in particular DNA, the 5′ end is always on the left-hand side.

| | | |
|---|---|---|
| 5′ end | 5′--P-NNNNNNN-OH-3′ | 3′ end |
| | 3′-HO-NNNNNNN-P--5′ | |

“Upstream” describes the relative positioning of a first element of a nucleic acid molecule with respect to a second element of that nucleic acid molecule, wherein both elements are comprised in the same nucleic acid molecule, and wherein the first element is located nearer to the 5′ end of the nucleic acid molecule than the second element of that nucleic acid molecule. The second element is then said to be “downstream” of the first element of that nucleic acid molecule. An element that is located “upstream” of a second element can be synonymously referred to as being located “5′” of that second element. For a double-stranded nucleic acid molecule, indications like “upstream” and “downstream” are given with respect to the (+) strand.

According to the invention, “functional linkage” or “functionally linked” relates to a connection within a functional relationship. A nucleic acid is “functionally linked” if it is functionally related to another nucleic acid sequence. For example, a promoter is functionally linked to a coding sequence if it influences transcription of said coding sequence. Functionally linked nucleic acids are typically adjacent to one another, where appropriate separated by further nucleic acid sequences, and, in particular embodiments, are transcribed by RNA polymerase to give a single RNA molecule (common transcript).

In particular embodiments, a nucleic acid is functionally linked according to the invention to expression control sequences which may be homologous or heterologous with respect to the nucleic acid.

The term “expression control sequence” comprises according to the invention promoters, ribosome-binding sequences and other control elements which control transcription of a gene or translation of the derived RNA. In particular embodiments of the invention, the expression control sequences can be regulated. The precise structure of expression control sequences may vary depending on the species or cell type but usually includes 5′-untranscribed and 5′- and 3′-untranslated sequences involved in initiating transcription and translation, respectively. More specifically, 5′-untranscribed expression control sequences include a promoter region which encompasses a promoter sequence for transcription control of the functionally linked gene. Expression control sequences may also include enhancer sequences or upstream activator sequences. An expression control sequence of a DNA molecule usually includes 5′-untranscribed and 5′- and 3′-untranslated sequences such as TATA box, capping sequence, CAAT sequence and the like. An expression control sequence of alphaviral RNA may include a subgenomic promoter and/or one or more conserved sequence element(s). A specific expression control sequence according to the present invention is a subgenomic promoter of an alphavirus, as described herein.

The nucleic acid sequences specified herein, in particular transcribable and coding nucleic acid sequences, may be combined with any expression control sequences, in particular promoters, which may be homologous or heterologous to said nucleic acid sequences, with the term “homologous” referring to the fact that a nucleic acid sequence is also functionally linked naturally to the expression control sequence, and the term “heterologous” referring to the fact that a nucleic acid sequence is not naturally functionally linked to the expression control sequence.

A transcribable nucleic acid sequence, in particular a nucleic acid sequence coding for a peptide or protein, and an expression control sequence are “functionally” linked to one another, if they are covalently linked to one another in such a way that transcription or expression of the transcribable and in particular coding nucleic acid sequence is under the control or under the influence of the expression control sequence. If the nucleic acid sequence is to be translated into a functional peptide or protein, induction of an expression control sequence functionally linked to the coding sequence results in transcription of said coding sequence, without causing a frame shift in the coding sequence or the coding sequence being unable to be translated into the desired peptide or protein.

The term “promoter” or “promoter region” refers to a nucleic acid sequence which controls synthesis of a transcript, e.g. a transcript comprising a coding sequence, by providing a recognition and binding site for RNA polymerase. The promoter region may include further recognition or binding sites for further factors involved in regulating transcription of said gene. A promoter may control transcription of a prokaryotic or eukaryotic gene. A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor. A specific promoter according to the present invention is a subgenomic promoter, e.g., of an alphavirus, as described herein. Other specific promoters are genomic plus-strand or negative-strand promoters, e.g., of an alphavirus.

The term “core promoter” refers to a nucleic acid sequence that is comprised by the promoter. The core promoter is typically the minimal portion of the promoter required to properly initiate transcription. The core promoter typically includes the transcription start site and a binding site for RNA polymerase.

A “polymerase” generally refers to a molecular entity capable of catalyzing the synthesis of a polymeric molecule from monomeric building blocks. An “RNA polymerase” is a molecular entity capable of catalyzing the synthesis of an RNA molecule from ribonucleotide building blocks. A “DNA polymerase” is a molecular entity capable of catalyzing the synthesis of a DNA molecule from deoxy ribonucleotide building blocks. For the case of DNA polymerases and RNA polymerases, the molecular entity is typically a protein or an assembly or complex of multiple proteins. Typically, a DNA polymerase synthesizes a DNA molecule based on a template nucleic acid, which is typically a DNA molecule. Typically, an RNA polymerase synthesizes an RNA molecule based on a template nucleic acid, which is either a DNA molecule (in that case the RNA polymerase is a DNA-dependent RNA polymerase, DdRP), or is an RNA molecule (in that case the RNA polymerase is an RNA-dependent RNA polymerase, RdRP).

An “RNA-dependent RNA polymerase” or “RdRP” or “replicase”, is an enzyme that catalyzes the transcription of RNA from an RNA template. In the case of alphaviral RNA-dependent RNA polymerase, sequential synthesis of (−) strand complement of genomic RNA and of (+) strand genomic RNA leads to RNA replication. RNA-dependent RNA polymerase is thus synonymously referred to as “RNA replicase” or simply “replicase”. In nature, RNA-dependent RNA polymerases are typically encoded by all RNA viruses except retroviruses. Typical representatives of viruses encoding an RNA-dependent RNA polymerase are alphaviruses.

According to the present invention, “RNA replication” generally refers to an RNA molecule synthesized based on the nucleotide sequence of a given RNA molecule (template RNA molecule). The RNA molecule that is synthesized may be, e.g., identical or complementary to the template RNA molecule. In general, RNA replication may occur via synthesis of a DNA intermediate, or may occur directly by RNA-dependent RNA replication mediated by an RNA-dependent RNA polymerase (RdRP). In the case of alphaviruses, RNA replication does not occur via a DNA intermediate, but is mediated by a RNA-dependent RNA polymerase (RdRP): a template RNA strand (first RNA strand)—or a part thereof—serves as template for the synthesis of a second RNA strand that is complementary to the first RNA strand or to a part thereof. The second RNA strand—or a part thereof—may in turn optionally serve as a template for synthesis of a third RNA strand that is complementary to the second RNA strand or to a part thereof. Thereby, the third RNA strand is identical to the first RNA strand or to a part thereof. Thus, RNA-dependent RNA polymerase is capable of directly synthesizing a complementary RNA strand of a template, and of indirectly synthesizing an identical RNA strand (via a complementary intermediate strand).

According to the invention, the term “template RNA” refers to RNA that can be transcribed or replicated by an RNA-dependent RNA polymerase.

According to the invention, the term “gene” refers to a particular nucleic acid sequence which is responsible for producing one or more cellular products and/or for achieving one or more intercellular or intracellular functions. More specifically, said term relates to a nucleic acid section (typically DNA; but RNA in the case of RNA viruses) which comprises a nucleic acid coding for a specific protein or a functional or structural RNA molecule.

An “isolated molecule” as used herein, is intended to refer to a molecule which is substantially free of other molecules such as other cellular material. The term “isolated nucleic acid” means according to the invention that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant techniques.

The term “vector” is used here in its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, virus genomes, and fractions thereof.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the invention, the term “expression” is used in its most general meaning and comprises production of RNA and/or protein. It also comprises partial expression of nucleic acids. Furthermore, expression may be transient or stable. With respect to RNA, the term “expression” or “translation” relates to the process in the ribosomes of a cell by which a strand of coding RNA (e.g. messenger RNA) directs the assembly of a sequence of amino acids to make a peptide or protein.

According to the invention, the term “mRNA” means “messenger-RNA” and relates to a transcript which is typically generated by using a DNA template and encodes a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to the skilled person. For example, there is a variety of in vitro transcription kits commercially available. According to the invention, mRNA may be modified by stabilizing modifications and capping.

According to the invention, the terms “poly(A) sequence” or “poly(A) tail” refer to an uninterrupted or interrupted sequence of adenylate residues which is typically located at the 3′ end of an RNA molecule. An uninterrupted sequence is characterized by consecutive adenylate residues. In nature, an uninterrupted poly(A) sequence is typical. While a poly(A) sequence is normally not encoded in eukaryotic DNA, but is attached during eukaryotic transcription in the cell nucleus to the free 3′ end of the RNA by a template-independent RNA polymerase after transcription, the present invention encompasses poly(A) sequences encoded by DNA.

According to the invention, the term “primary structure”, with reference to a nucleic acid molecule, refers to the linear sequence of nucleotide monomers.

According to the invention, the term “secondary structure”, with reference to a nucleic acid molecule, refers to a two-dimensional representation of a nucleic acid molecule that reflects base pairings; e.g.; in the case of a single-stranded RNA molecule particularly intramolecular base pairings. Although each RNA molecule has only a single polynucleotide chain, the molecule is typically characterized by regions of (intramolecular) base pairs. According to the invention, the term “secondary structure” comprises structural motifs including without limitation base pairs, stems, stem loops, bulges, loops such as interior loops and multi-branch loops. The secondary structure of a nucleic acid molecule can be represented by a two-dimensional drawing (planar graph), showing base pairings (for further details on secondary structure of RNA molecules, see Auber et al., 2006; J. Graph Algorithms Appl. 10:329-351). As described herein, the secondary structure of certain RNA molecules is relevant in the context of the present invention.

According to the invention, secondary structure of a nucleic acid molecule, particularly of a single-stranded RNA molecule, is determined by prediction using the web server for RNA secondary structure prediction (http://ma.urmc.rochester.edu/RNAstructureWeb/Servers/Predict1/Predict1.html). Preferably, according to the invention, “secondary structure”, with reference to a nucleic acid molecule, specifically refers to the secondary structure determined by said prediction. The prediction may also be performed or confirmed using MFOLD structure prediction (http://unafold.ma.albany.edu/?q=mfold).

According to the invention, a “base pair” is a structural motif of a secondary structure wherein two nucleotide bases associate with each other through hydrogen bonds between donor and acceptor sites on the bases. The complementary bases, A:U and G:C, form stable base pairs through hydrogen bonds between donor and acceptor sites on the bases; the A:U and G:C base pairs are called Watson-Crick base pairs. A weaker base pair (called Wobble base pair) is formed by the bases G and U (G:U). The base pairs A:U and G:C are called canonical base pairs. Other base pairs like G:U (which occurs fairly often in RNA) and other rare base-pairs (e.g. A:C; U:U) are called non-canonical base pairs.

According to the invention, “nucleotide pairing” refers to two nucleotides that associate with each other so that their bases form a base pair (canonical or non-canonical base pair, preferably canonical base pair, most preferably Watson-Crick base pair).

According to the invention, the terms “stem loop” or “hairpin” or “hairpin loop”, with reference to a nucleic acid molecule, all interchangeably refer to a particular secondary structure of a nucleic acid molecule, typically a single-stranded nucleic acid molecule, such as single-stranded RNA. The particular secondary structure represented by the stem loop consists of a consecutive nucleic acid sequence comprising a stem and a (terminal) loop, also called hairpin loop, wherein the stem is formed by two neighbored entirely or partially complementary sequence elements; which are separated by a short sequence (e.g. 3-10 nucleotides), which forms the loop of the stem-loop structure. The two neighbored entirely or partially complementary sequences may be defined as, e.g., stem loop elements stem 1 and stem 2. The stem loop is formed when these two neighbored entirely or partially reverse complementary sequences, e.g. stem loop elements stem 1 and stem 2, form base-pairs with each other, leading to a double stranded nucleic acid sequence comprising an unpaired loop at its terminal ending formed by the short sequence located between stem loop elements stem 1 and stem 2. Thus, a stem loop comprises two stems (stem 1 and stem 2), which—at the level of secondary structure of the nucleic acid molecule—form base pairs with each other, and which—at the level of the primary structure of the nucleic acid molecule—are separated by a short sequence that is not part of stem 1 or stem 2. For illustration, a two-dimensional representation of the stem loop resembles a lollipop-shaped structure. The formation of a stem-loop structure requires the presence of a sequence that can fold back on itself to form a paired double strand; the paired double strand is formed by stem 1 and stem 2. The stability of paired stem loop elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). According to the present invention, the optimal loop length is 3-10 nucleotides, more preferably 4 to 7, nucleotides, such as 4 nucleotides, 5 nucleotides, 6 nucleotides or 7 nucleotides. If a given nucleic acid sequence is characterized by a stem loop, the respective complementary nucleic acid sequence is typically also characterized by a stem loop. A stem loop is typically formed by single-stranded RNA molecules. For example, several stem loops are present in the 5′ replication recognition sequence of alphaviral genomic RNA.

According to the invention, “disruption” or “disrupt”, with reference to a specific secondary structure of a nucleic acid molecule (e.g., a stem loop) means that the specific secondary structure is absent or altered. Typically, a secondary structure may be disrupted as a consequence of a change of at least one nucleotide that is part of the secondary structure. For example, a stem loop may be disrupted by change of one or more nucleotides that form the stem, so that nucleotide pairing is not possible.

According to the invention, “compensates for secondary structure disruption” or “compensating for secondary structure disruption” refers to one or more nucleotide changes in a nucleic acid sequence; more typically it refers to one or more second nucleotide changes in a nucleic acid sequence, which nucleic acid sequence also comprises one or more first nucleotide changes, characterized as follows: while the one or more first nucleotide changes, in the absence of the one or more second nucleotide changes, cause a disruption of the secondary structure of the nucleic acid sequence, the co-occurrence of the one or more first nucleotide changes and the one or more second nucleotide changes does not cause the secondary structure of the nucleic acid to be disrupted. Co-occurrence means presence of both the one or more first nucleotide changes and of the one or more second nucleotide changes. Typically, the one or more first nucleotide changes and the one or more second nucleotide changes are present together in the same nucleic acid molecule. In a specific embodiment, one or more nucleotide changes that compensate for secondary structure disruption is/are one or more nucleotide changes that compensate for one or more nucleotide pairing disruptions. Thus, in one embodiment, “compensating for secondary structure disruption” means “compensating for nucleotide pairing disruptions”, i.e. one or more nucleotide pairing disruptions, for example one or more nucleotide pairing disruptions within one or more stem loops. The one or more one or more nucleotide pairing disruptions may have been introduced by the removal of at least one initiation codon. Each of the one or more nucleotide changes that compensates for secondary structure disruption is a nucleotide change, which can each be independently selected from a deletion, an addition, a substitution and/or an insertion of one or more nucleotides. In an illustrative example, when the nucleotide pairing A:U has been disrupted by substitution of A to C (C and U are not typically suitable to form a nucleotide pair); then a nucleotide change that compensates for nucleotide pairing disruption may be substitution of U by G, thereby enabling formation of the C:G nucleotide pairing. The substitution of U by G thus compensates for the nucleotide pairing disruption. In an alternative example, when the nucleotide pairing A:U has been disrupted by substitution of A to C; then a nucleotide change that compensates for nucleotide pairing disruption may be substitution of C by A, thereby restoring formation of the original A:U nucleotide pairing. In general, in the present invention, those nucleotide changes compensating for secondary structure disruption are preferred which do neither restore the original nucleic acid sequence nor create novel AUG triplets. In the above set of examples, the U to G substitution is preferred over the C to A substitution.

According to the invention, the term “tertiary structure”, with reference to a nucleic acid molecule, refers to the three-dimensional structure of a nucleic acid molecule, as defined by the atomic coordinates.

According to the invention, a nucleic acid such as RNA, e.g., rRNA, may encode a peptide or protein. Accordingly, a transcribable nucleic acid sequence or a transcript thereof may contain an open reading frame (ORF) encoding a peptide or protein.

According to the invention, the term “nucleic acid encoding a peptide or protein” means that the nucleic acid, if present in the appropriate environment, preferably within a cell, can direct the assembly of amino acids to produce the peptide or protein during the process of translation. Preferably, coding RNA according to the invention is able to interact with the cellular translation machinery allowing translation of the coding RNA to yield a peptide or protein.

According to the invention, the term “peptide” comprises oligo- and polypeptides and refers to substances which comprise two or more, preferably 3 or more, preferably 4 or more, preferably 6 or more, preferably 8 or more, preferably 10 or more, preferably 13 or more, preferably 16 or more, preferably 20 or more, and up to preferably 50, preferably 100 or preferably 150, consecutive amino acids linked to one another via peptide bonds. The term “protein” refers to large peptides, preferably peptides having at least 151 amino acids, but the terms “peptide” and “protein” are used herein usually as synonyms.

The terms “peptide” and “protein” comprise, according to the invention, substances which contain not only amino acid components but also non-amino acid components such as sugars and phosphate structures, and also comprise substances containing bonds such as ester, thioether or disulfide bonds.

According to the invention, the terms “initiation codon” and “start codon” synonymously refer to a codon (base triplet) of an RNA molecule that is potentially the first codon that is translated by a ribosome. Such codon typically encodes the amino acid methionine in eukaryotes and a modified methionine in prokaryotes. The most common initiation codon in eukaryotes and prokaryotes is AUG. Unless specifically stated herein that an initiation codon other than AUG is meant, the terms “initiation codon” and “start codon”, with reference to an RNA molecule, refer to the codon AUG. According to the invention, the terms “initiation codon” and “start codon” are also used to refer to a corresponding base triplet of a deoxyribonucleic acid, namely the base triplet encoding the initiation codon of an RNA. If the initiation codon of messenger RNA is AUG, the base triplet encoding the AUG is ATG. According to the invention, the terms “initiation codon” and “start codon” preferably refer to a functional initiation codon or start codon, i.e., to an initiation codon or start codon that is used or would be used as a codon by a ribosome to start translation. There may be AUG codons in an RNA molecule that are not used as codons by a ribosome to start translation, e.g., due to a short distance of the codons to the cap. These codons are not encompassed by the term functional initiation codon or start codon.

According to the invention, the terms “start codon of the open reading frame” or “initiation codon of the open reading frame” refer to the base triplet that serves as initiation codon for protein synthesis in a coding sequence, e.g., in the coding sequence of a nucleic acid molecule found in nature. In an RNA molecule, the start codon of the open reading frame is often preceded by a 5′ untranslated region (5′-UTR), although this is not strictly required.

According to the invention, the terms “native start codon of the open reading frame” or “native initiation codon of the open reading frame” refer to the base triplet that serves as initiation codon for protein synthesis in a native coding sequence. A native coding sequence may be, e.g., the coding sequence of a nucleic acid molecule found in nature. In some embodiments, the present invention provides variants of nucleic acid molecules found in nature, which are characterized in that the native start codon (which is present in the native coding sequence) has been removed (so that it is not present in the variant nucleic acid molecule).

According to the invention, “first AUG” means the most upstream AUG base triplet of a messenger RNA molecule, preferably the most upstream AUG base triplet of a messenger RNA molecule that is used or would be used as a codon by a ribosome to start translation. Accordingly, “first ATG” refers to the ATG base triplet of a coding DNA sequence that encodes the first AUG. In some instances, the first AUG of a mRNA molecule is the start codon of an open reading frame, i.e., the codon that is used as start codon during ribosomal protein synthesis.

According to the invention, the terms “comprises the removal” or “characterized by the removal” and similar terms, with reference to a certain element of a nucleic acid variant, mean that said certain element is not functional or not present in the nucleic acid variant, compared to a reference nucleic acid molecule. Without limitation, a removal can consist of deletion of all or part of the certain element, of substitution of all or part of the certain element, or of alteration of the functional or structural properties of the certain element. The removal of a functional element of a nucleic acid sequence requires that the function is not exhibited at the position of the nucleic acid variant comprising the removal. For example, an RNA variant characterized by the removal of a certain initiation codon requires that ribosomal protein synthesis is not initiated at the position of the RNA variant characterized by the removal. The removal of a structural element of a nucleic acid sequence requires that the structural element is not present at the position of the nucleic acid variant comprising the removal. For example, a RNA variant characterized by the removal of a certain AUG base triplet, i.e., of a AUG base triplet at a certain position, may be characterized, e.g., by deletion of part or all of the certain AUG base triplet (e.g., ΔAUG), or by substitution of one or more nucleotides (A, U, G) of the certain AUG base triplet by any one or more different nucleotides, so that the resulting nucleotide sequence of the variant does not comprise said AUG base triplet. Suitable substitutions of one nucleotide are those that convert the AUG base triplet into a GUG, CUG or UUG base triplet, or into a AAG, ACG or AGG base triplet, or into a AUA, AUC or AUU base triplet. Suitable substitutions of more nucleotides can be selected accordingly.

According to the invention, the term “self-replicating virus” includes RNA viruses capable of replicating autonomously in a host cell. Self-replicating viruses may have a single-stranded RNA (ssRNA) genome and include alphaviruses, flaviviruses, measles viruses (MVs) and rhabdoviruses. Alphaviruses and flaviviruses possess a genome of positive polarity, whereas the genome of measles viruses (MVs) and rhabdoviruses is negative strand ssRNA. Typically, a self-replicating virus is a virus with a (+) stranded RNA genome which can be directly translated after infection of a cell, and this translation provides an RNA-dependent RNA polymerase which then produces both antisense and sense transcripts from the infected RNA. In the following, the invention is illustrated by referring to alphavirus-derived vectors as an example of self-replicating virus-derived vectors. However, it is to be understood that the present invention is not limited to alphavirus-derived vectors.

According to the invention, the term “alphavirus” is to be understood broadly and includes any virus particle that has characteristics of alphaviruses. Characteristics of alphavirus include the presence of a (+) stranded RNA which encodes genetic information suitable for replication in a host cell, including RNA polymerase activity. Further characteristics of many alphaviruses are described, e.g., in Strauss & Strauss, 1994, Microbiol. Rev. 58:491-562. The term “alphavirus” includes alphavirus found in nature, as well as any variant or derivative thereof. In some embodiments, a variant or derivative is not found in nature.

In one embodiment, the alphavirus is an alphavirus found in nature. Typically, an alphavirus found in nature is infectious to any one or more eukaryotic organisms, such as an animal (including a vertebrate such as a human, and an arthropod such as an insect). An alphavirus found in nature is preferably selected from the group consisting of the following: Barmah Forest virus complex (comprising Barmah Forest virus); Eastern equine encephalitis complex (comprising seven antigenic types of Eastern equine encephalitis virus); Middelburg virus complex (comprising Middelburg virus); Ndumu virus complex (comprising Ndumu virus); Semliki Forest virus complex (comprising Bebaru virus, Chikungunya virus, Mayaro virus and its subtype Una virus, O'Nyong Nyong virus, and its subtype Igbo-Ora virus, Ross River virus and its subtypes Bebaru virus, Getah virus, Sagiyama virus, Semliki Forest virus and its subtype Me Tri virus); Venezuelan equine encephalitis complex (comprising Cabassou virus, Everglades virus, Mosso das Pedras virus, Mucambo virus, Paramana virus, Pixuna virus, Rio Negro virus, Trocara virus and its subtype Bijou Bridge virus, Venezuelan equine encephalitis virus); Western equine encephalitis complex (comprising Aura virus, Babanki virus, Kyzylagach virus, Sindbis virus, Ockelbo virus, Whataroa virus, Buggy Creek virus, Fort Morgan virus, Highlands J virus, Western equine encephalitis virus); and some unclassified viruses including Salmon pancreatic disease virus; Sleeping Disease virus; Southern elephant seal virus; Tonate virus. More preferably, the alphavirus is selected from the group consisting of Semliki Forest virus complex (comprising the virus types as indicated above, including Semliki Forest virus), Western equine encephalitis complex (comprising the virus types as indicated above, including Sindbis virus), Eastern equine encephalitis virus (comprising the virus types as indicated above), Venezuelan equine encephalitis complex (comprising the virus types as indicated above, including Venezuelan equine encephalitis virus).

In a further preferred embodiment, the alphavirus is Semliki Forest virus. In an alternative further preferred embodiment, the alphavirus is Sindbis virus. In an alternative further preferred embodiment, the alphavirus is Venezuelan equine encephalitis virus.

In some embodiments of the present invention, the alphavirus is not an alphavirus found in nature. Typically, an alphavirus not found in nature is a variant or derivative of an alphavirus found in nature, that is distinguished from an alphavirus found in nature by at least one mutation in the nucleotide sequence, i.e., the genomic RNA. The mutation in the nucleotide sequence may be selected from an insertion, a substitution or a deletion of one or more nucleotides, compared to an alphavirus found in nature. A mutation in the nucleotide sequence may or may not be associated with a mutation in a polypeptide or protein encoded by the nucleotide sequence. For example, an alphavirus not found in nature may be an attenuated alphavirus. An attenuated alphavirus not found in nature is an alphavirus that typically has at least one mutation in its nucleotide sequence by which it is distinguished from an alphavirus found in nature, and that is either not infectious at all, or that is infectious but has a lower disease-producing ability or no disease-producing ability at all. As an illustrative example, TC83 is an attenuated alphavirus that is distinguished from the Venezuelan equine encephalitis virus (VEEV) found in nature (McKinney et al., 1963, Am. J. Trop. Med. Hyg. 12:597-603).

Members of the alphavirus genus may also be classified based on their relative clinical features in humans: alphaviruses associated primarily with encephalitis, and alphaviruses associated primarily with fever, rash, and polyarthritis.

The term “alphaviral” means found in an alphavirus, or originating from an alphavirus or derived from an alphavirus, e.g., by genetic engineering.

According to the invention, “SFV” stands for Semliki Forest virus. According to the invention, “SIN” or “SINV” stands for Sindbis virus. According to the invention, “VEE” or “VEEV” stands for Venezuelan equine encephalitis virus.

According to the invention, the term “of an alphavirus” refers to an entity of origin from an alphavirus. For illustration, a protein of an alphavirus may refer to a protein that is found in alphavirus and/or to a protein that is encoded by alphavirus; and a nucleic acid sequence of an alphavirus may refer to a nucleic acid sequence that is found in alphavirus and/or to a nucleic acid sequence that is encoded by alphavirus. Preferably, a nucleic acid sequence “of an alphavirus” refers to a nucleic acid sequence “of the genome of an alphavirus” and/or “of genomic RNA of an alphavirus”.

According to the invention, the term “alphaviral RNA” refers to any one or more of alphaviral genomic RNA (i.e., (+) strand), complement of alphaviral genomic RNA (i.e., (−) strand), and the subgenomic transcript (i.e. (+) strand), or a fragment of any thereof.

According to the invention, “alphavirus genome” refers to genomic (+) strand RNA of an alphavirus.

According to the invention, the term “native alphavirus sequence” and similar terms typically refer to a (e.g., nucleic acid) sequence of a naturally occurring alphavirus (alphavirus found in nature). In some embodiments, the term “native alphavirus sequence” also includes a sequence of an attenuated alphavirus.

According to the invention, the term “5′ replication recognition sequence” preferably refers to a continuous nucleic acid sequence, preferably a ribonucleic acid sequence, that is identical or homologous to a 5′ fragment of a genome of a self-replicating virus, such as an alphavirus genome. The “5′ replication recognition sequence” is a nucleic acid sequence that can be recognized by a replicase such as an alphaviral replicase. The term 5′ replication recognition sequence includes native 5′ replication recognition sequences as well as functional equivalents thereof, such as, e.g., functional variants of a 5′ replication recognition sequence of a self-replicating virus found in nature, e.g., alphavirus found in nature. According to the invention, functional equivalents include derivatives of 5′ replication recognition sequences characterized by the removal of at least one initiation codon as described herein. The 5′ replication recognition sequence is required for synthesis of the (−) strand complement of alphavirus genomic RNA, and is required for synthesis of (+) strand viral genomic RNA based on a (−) strand template. A native 5′ replication recognition sequence typically encodes at least the N-terminal fragment of nsP1; but does not comprise the entire open reading frame encoding nsP1234. In view of the fact that a native 5′ replication recognition sequence typically encodes at least the N-terminal fragment of nsP1, a native 5′ replication recognition sequence typically comprises at least one initiation codon, typically AUG. In one embodiment, the 5′ replication recognition sequence comprises conserved sequence element 1 of an alphavirus genome (CSE 1) or a variant thereof and conserved sequence element 2 of an alphavirus genome (CSE 2) or a variant thereof. The 5′ replication recognition sequence is typically capable of forming four stem loops (SL), i.e. SL1, SL2, SL3, SL4. The numbering of these stem loops begins at the 5′ end of the 5′ replication recognition sequence.

The term “conserved sequence element” or “CSE” refers to a nucleotide sequence found in alphavirus RNA. These sequence elements are termed “conserved” because orthologs are present in the genome of different alphaviruses, and orthologous CSEs of different alphaviruses preferably share a high percentage of sequence identity and/or a similar secondary or tertiary structure. The term CSE includes CSE 1, CSE 2, CSE 3 and CSE 4.

According to the invention, the terms “CSE 1” or “44-nt CSE” synonymously refer to a nucleotide sequence that is required for (+) strand synthesis from a (−) strand template. The term “CSE 1” refers to a sequence on the (+) strand; and the complementary sequence of CSE 1 (on the (−) strand) functions as a promoter for (+) strand synthesis. Preferably, the term CSE 1 includes the most 5′ nucleotide of the alphavirus genome. CSE 1 typically forms a conserved stem-loop structure. Without wishing to be bound to a particular theory, it is believed that, for CSE 1, the secondary structure is more important than the primary structure, i.e., the linear sequence. In genomic RNA of the model alphavirus Sindbis virus, CSE 1 consists of a consecutive sequence of 44 nucleotides, which is formed by the most 5′ 44 nucleotides of the genomic RNA (Strauss & Strauss, 1994, Microbiol. Rev. 58:491-562).

According to the invention, the terms “CSE 2” or “51-nt CSE” synonymously refer to a nucleotide sequence that is required for (−) strand synthesis from a (+) strand template. The (+) strand template is typically alphavirus genomic RNA or an RNA replicon (note that the subgenomic RNA transcript, which does not comprise CSE 2, does not function as a template for (−) strand synthesis). In alphavirus genomic RNA, CSE 2 is typically localized within the coding sequence for nsP1. In genomic RNA of the model alphavirus Sindbis virus, the 51-nt CSE is located at nucleotide positions 155-205 of genomic RNA (Frolov et al., 2001, RNA, vol. 7, pp. 1638-1651). CSE 2 forms typically two conserved stem loop structures. These stem loop structures are designated as stem loop 3 (SL3) and stem loop 4 (SL4) because they are the third and fourth conserved stem loop, respectively, of alphavirus genomic RNA, counted from the 5′ end of alphavirus genomic RNA. Without wishing to be bound to a particular theory, it is believed that, for CSE 2, the secondary structure is more important than the primary structure, i.e. the linear sequence.

According to the invention, the terms “CSE 3” or “junction sequence” synonymously refer to a nucleotide sequence that is derived from alphaviral genomic RNA and that comprises the start site of the subgenomic RNA. The complement of this sequence in the (−) strand acts to promote subgenomic RNA transcription. In alphavirus genomic RNA, CSE 3 typically overlaps with the region encoding the C-terminal fragment of nsP4 and extends to a short non-coding region located upstream of the open reading frame encoding the structural proteins.

According to the invention, the terms “CSE 4” or “19-nt conserved sequence” or “19-nt CSE” synonymously refer to a nucleotide sequence from alphaviral genomic RNA, immediately upstream of the poly(A) sequence in the 3′ untranslated region of the alphavirus genome. CSE 4 typically consists of 19 consecutive nucleotides. Without wishing to be bound to a particular theory, CSE 4 is understood to function as a core promoter for initiation of (−) strand synthesis (Jos6 et al., 2009, Future Microbiol. 4:837-856); and/or CSE 4 and the poly(A) tail of the alphavirus genomic RNA are understood to function together for efficient (−) strand synthesis (Hardy & Rice, 2005, J. Virol. 79:4630-4639).

According to the invention, the term “subgenomic promoter” or “SGP” refers to a nucleic acid sequence upstream (5′) of a nucleic acid sequence (e.g., coding sequence), which controls transcription of said nucleic acid sequence by providing a recognition and binding site for RNA polymerase, typically RNA-dependent RNA polymerase, in particular functional alphavirus non-structural protein. The SGP may include further recognition or binding sites for further factors. A subgenomic promoter is typically a genetic element of a positive strand RNA virus, such as an alphavirus. A subgenomic promoter of alphavirus is a nucleic acid sequence comprised in the viral genomic RNA. The subgenomic promoter is generally characterized in that it allows initiation of the transcription (RNA synthesis) in the presence of an RNA-dependent RNA polymerase, e.g., functional alphavirus non-structural protein. An RNA (−) strand, i.e., the complement of alphaviral genomic RNA, serves as a template for synthesis of a (+) strand subgenomic transcript, and synthesis of the (+) strand subgenomic transcript is typically initiated at or near the subgenomic promoter. The term “subgenomic promoter” as used herein, is not confined to any particular localization in a nucleic acid comprising such subgenomic promoter. In some embodiments, the SGP is identical to CSE 3 or overlaps with CSE 3 or comprises CSE 3.

The terms “subgenomic transcript” or “subgenomic RNA” synonymously refer to an RNA molecule that is obtainable as a result of transcription using a RNA molecule as template (“template RNA”), wherein the template RNA comprises a subgenomic promoter that controls transcription of the subgenomic transcript. The subgenomic transcript is obtainable in the presence of an RNA-dependent RNA polymerase, in particular functional alphavirus non-structural protein. For instance, the term “subgenomic transcript” may refer to the RNA transcript that is prepared in a cell infected by an alphavirus, using the (−) strand complement of alphavirus genomic RNA as template. However, the term “subgenomic transcript”, as used herein, is not limited thereto and also includes transcripts obtainable by using heterologous RNA as template. For example, subgenomic transcripts are also obtainable by using the (−) strand complement of SGP-containing replicons according to the present invention as template. Thus, the term “subgenomic transcript” may refer to an RNA molecule that is obtainable by transcribing a fragment of alphavirus genomic RNA, as well as to an RNA molecule that is obtainable by transcribing a fragment of a replicon according to the present invention.

The term “autologous” is used to describe anything that is derived from the same subject. For example, “autologous cell” refers to a cell derived from the same subject. Introduction of autologous cells into a subject is advantageous because these cells overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues or cells.

The term “heterologous” is used to describe something consisting of multiple different elements. As an example, the introduction of one individual's cell into a different individual constitutes a heterologous transplant. A heterologous gene is a gene derived from a source other than the subject.

Cells that may be used in the methods for identifying sequence changes are any appropriate cell in which the rRNA, with or without any nucleotide modifications, can be replicated and/or translated. The cell may be a mammalian cell, for example, a human cell. The cell may constitutively express a replicase which recognizes the sequences present in the rRNA for replication or may transiently express such replicase.

The following provides specific and/or preferred variants of the individual features of the invention. The present invention also contemplates as particularly preferred embodiments those embodiments, which are generated by combining two or more of the specific and/or preferred variants described for two or more of the features of the present invention.

### RNA Replicon

A replicable RNA (rRNA) is an RNA that is able to be replicated by an RNA-dependent RNA polymerase (replicase) by virtue of comprising nucleotide sequences that can be recognized by the replicase such that the RNA is replicated. The rRNA does not necessarily encode the replicase, such that rRNAs can be replicated in cis (by the encoded replicase) or in trans (by a replicase provided in another manner, e.g., a separate replicase encoding nucleic acid). The terms “RNA replicon”, “replicon” and “replicable RNA molecule” can be used interchangeably.

In an embodiment, the replicable RNA (rRNA) molecule comprises a modified regulatory region of a self-replicating single-stranded positive-sense virus comprising sequence changes compared to a reference modified regulatory region, which sequence changes restore or improve the function of the rRNA molecule that comprises at least one modified nucleotide. These changes may be identified by the methods described herein for identifying such sequence changes. In an embodiment, the modified regulatory region is an alphavirus regulatory region, e.g., a 5′ or a 3′ regulatory region. In an embodiment, the sequence changes of an alphavirus 5′ regulatory region comprise a point mutation at one or more positions corresponding to positions 67, 244, 245, 246, 248 of SEQ ID NO:2. In an embodiment, the 5′ regulatory region is the VEEV alphaviral 5′ regulatory region. In an embodiment, the 5′ regulatory region has the sequence depicted in SEQ ID NO: 1 or 2. In an embodiment, the 5′ regulatory region comprises a point mutation at one or more of positions 67, 244, 245, 246, 248 of the VEEV 5′ regulatory region having the sequence depicted in SEQ ID NO: 2 or to positions in the 5′ regulatory region of another alphavirus corresponding to those positions in SEQ ID NO: 2. In an embodiment, the rRNA molecule further comprises in the 5′ regulatory region a point mutation at position 4 of SEQ ID NO: 2.

In various embodiments, the point mutation may be at position 67, at positions 67 and 244, at positions 67 and 246, at positions 67 and 248, at positions 67, 245 and 248, at positions 4 and 67, at positions 4, 67 and 244, at positions 4, 67 and 246, at positions 4, 67 and 248, at positions 4, 67, 245 and 248 of SEQ ID NO: 2, or any combination thereof, or at positions in alphaviruses corresponding to such positions in SEQ ID NO: 2.

In various embodiments, the point mutation may be at one or more of the following positions or combination of positions of SEQ ID NO: 2 or at corresponding positions in the 5′ regulatory region of an alphavirus:
67; 244; 245; 246; 248; 67 and 244; 67 and 245; 67 and 246; 67 and 248; 244 and 245; 244 and 246; 244 and 248; 245 and 246; 245 and 248; 246 and 248; 67, 244 and 245; 67, 244 and 246; 67, 244 and 248; 67, 245 and 246; 67, 245 and 248; 67, 246 and 248; 244, 245 and 246; 244, 245 and 248; 244, 246 and 248; 245, 246 and 248; 67, 244, 245 and 246; 67, 244, 245 and 248; 67, 244, 246 and 248; 67, 245, 246 and 248; 244, 245, 246 and 248; 67, 244, 245, 246 and 248. In each of the foregoing positions or combination of positions, at point mutation further may be at position 4 of SEQ ID NO: 2 or at the corresponding position in the 5′ regulatory region of an alphavirus.

In an embodiment, a specific point mutation at the various positions may be G4A, A67C, G244A, C245A, G246A, or C248A.

In an embodiment, an RNA replicon may comprise an internal ribosome entry site (IRES) and an open reading frame encoding a functional non-structural protein from a self-replicating virus, wherein the IRES controls expression of the functional non-structural protein, e.g., a replicase. Preferably, the RNA replicon contains sequence elements allowing replication by the functional non-structural protein. In one embodiment, the self-replicating virus is an alphavirus and the sequence elements allowing replication by the functional non-structural protein are derived from an alphavirus.

Alphavirus replicases have a capping enzyme function, and, typically, genomic as well as subgenomic (+) stranded RNAs are capped. The 5′-cap serves to protect mRNA from degradation, and to direct the ribosomal subunits as well as cellular factors to the mRNA in order to form a ribonucleoprotein complex on the mRNA that then can start translation from a nearby start codon. This complex process is extensively described in the literature (Jackson et al., 2010, Nat Rev Mol Biol; Vol 10:113-127). Despite the very elaborated and efficient mechanism of cap dependent translation, cells have means to initiate translation fully or partially independently from the 5′ cap (Thompson 2012; Trends in Microbiology 20:558-566). Thereby, in situations of cellular stress that lead to a global down regulation of cap-dependent translation, the cells may still express selected genes preferentially, often with the help of an IRES.

Viruses also evolved different means to exploit the cells machinery for translation of the viral genes. Since a viral infection is often sensed by the cell which leads to cellular antiviral response (interferon response; stress response), many viruses also make use of cap-independent translation, especially RNA viruses. Cap independent translation ensure an advantage for the viral RNA translation upon cellular stress response giving the viruses the opportunity to fulfil their life cycle and be released from infected cells.

Internal ribosomal entry sites (IRESs) are RNA sequences forming appropriate secondary structures that attract the pre-initiation complex near to a translational start codon, AUG or others. Four classes of IRESs are described in literature that share common features. Prototypic IRESs are the poliovirus IRES (Type I), the encephalomyocarditis virus (EMCV) IRES (Type II), the hepathitis C virus (HCV) IRES (Type III) and the IRES found in the intergenic regions of dicistroviruses (Type IV) (Thompson, 2012; Trends in Microbiology 20:558-566; Lozano et al., 2018; Open Biology 8:180155).

Type I to III IRESs have in common that they initiate translation at AUG start codons, whereas type IV IRES initiate at non-AUG codons (e.g., GCU). Thereby Type I to III require the initiator tRNA that delivers methionin by the help of eIF2/GTP (eIF2/GTP/Met-tRNAiMet). Activation of eIF2 kinases under stress phosphorylates the alpha subunit of eIF2 which inhibits translation that initiates at AUG. Thereby translation directed by type IV IRESs are not inhibited by eIF2 phosphorylation.

The term “internal ribosome entry site”, abbreviated “IRES”, relates to an RNA element that recruits ribosomes to the internal region of mRNAs to initiate translation in a cap-independent manner. IRESs are commonly located in the 5′-UTR of RNA viruses. However, mRNAs of viruses from dicistroviridae family possess two open reading frames (ORFs), and translation of each is directed by two distinct IRESs. It has also been suggested that some mammalian cellular mRNAs also have IRESs. These cellular IRES elements are thought to be located in eukaryotic mRNAs encoding genes involved in stress survival, and other processes critical to survival. The location for IRES elements is often in the 5′-UTR, but can also occur elsewhere in mRNAs.

The term “internal ribosome entry site” includes IRESs that are present in the viruses of the Picornaviridae family such as poliovirus (PV) and encephalomyocarditis virus and pathogenic viruses, including human immunodeficiency virus, hepatitis C virus (HCV), and foot and mouth disease virus. Although these viral IRESs contain diverse sequences, many of them have similar secondary structures and initiate translation through similar mechanisms. In addition, the activities of IRESs often require assistance from other factors known as IRES-transacting factors (ITAFs). Based on the structures and the requirement of translation initiation factors (IFs) and ITAFs, the viral IRESs are classified into four types as described herein. Any of these IRES types is useful according to the invention, with Type IV IRESs being particularly preferred.

Two groups of viral IRESs, Type I and Type II, cannot bind to the 40S small ribosomal subunit directly. Instead, they recruit the 40S small ribosomal subunit through different ITAFs and require canonical IFs in the cap-dependent translation (i.e., eIF2, eIF3, eIF4A, eIF4B, and eIF4G). The major difference between Type I and Type II IRESs is the requirement of 40S ribosome scanning, with 40S ribosome scanning being unnecessary for Type II IRES. Examples of Type I IRESs include IRESs found in poliovirus (PV) and rhinovirus. Examples of Type II IRESs include IRESs found in encephalomyocarditis virus (EMCV), foot-and-mouth disease virus (FMDV) and Theiler's murine encephalomyelitis viruses (TMEV).

Type III IRESs can directly interact with 40S small ribosomal subunit with specialized RNA structure, but their activities usually require assistance of several IFs including eIF2 and eIF3 and initiator Met-tRNAi. Examples include IRESs found in hepatitis C-virus (HCV), classical swine fever virus (CSFV) and porcine teschovirus (PTV).

Type IV viral IRESs generally have strong activities and can initiate translation from a non-AUG start codon without additional ITAFs or even eIF2/Met-tRNAi/GTP ternary complex. These IRESs are folded to a compact structure that directly interacts with the 40S small ribosomal subunit. Examples include IRESs found in dicistroviruses such as cricket paralysis virus (CrPV), plautia stali intestine virus (PSIV), and Taura-Syndrom-Virus (TSV).

The term “internal ribosome entry site” also includes IRESs found in cellular mRNAs, many of which encode proteins required in stress response, e.g. in conditions of apoptosis, mitosis, hypoxia, and nutrient limitation. The cellular IRESs can be roughly classified into two types based on the mechanisms of ribosome recruitment: Type I IRESs interact with ribosomes through ITAFs that bound on the cis-elements, e.g., RNA binding motifs and N-6-methyladenosine (m6A) modification, whereas Type II IRESs contain a short cis-element that pairs with 18S rRNA to recruit ribosomes.

In an embodiment, the rRNA described herein may have modified nucleotides/nucleosides/backbone modifications. The term “RNA modification” as used herein may refer to chemical modifications comprising backbone modifications as well as sugar modifications or base modifications.

In this context, a modified rRNA molecule as defined herein may contain nucleotide analogues/modifications, e.g., backbone modifications, sugar modifications or base modifications. A backbone modification in connection with the present invention is a modification, in which phosphates of the backbone of the nucleotides contained in an rRNA molecule as defined herein are chemically modified. A sugar modification in connection with the present invention is a chemical modification of the sugar of the nucleotides of the rRNA molecule as defined herein. Furthermore, a base modification in connection with the present invention is a chemical modification of the base moiety of the nucleotides of the rRNA molecule. In this context, nucleotide analogues or modifications are preferably selected from nucleotide analogues, which are applicable for transcription and/or translation.

Sugar Modifications: The modified nucleosides and nucleotides, which may be incorporated into a modified rRNA molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R=H, alkyl, cycloalkyl, aryl, aralkyl, heteroaryl or sugar); polyethyleneglycols (PEG), -0(CH2CH2 0)nCH2CH2 OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

Backbone Modifications: The phosphate backbone may further be modified in the modified nucleosides and nucleotides, which may be incorporated into a modified RNA molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications: The modified nucleosides and nucleotides, which may be incorporated into a modified rRNA molecule as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particular embodiments of the present invention, the nucleotide analogues/modifications are selected from base modifications, which are preferably selected from 2-amino-6-chloropurineriboside-5′-triphosphate, 2-aminopurine-riboside-5′-triphosphate; 2-aminoadenosine-5′-triphosphate, 2′-amino-2′-deoxy-cytidine-triphosphate, 2-thiocytidine-5′-triphosphate, 2-thiouridine-5′-triphosphate, 2′-fluorothymidine-5′-triphosphate, 2′-0-methyl inosine-5′-triphosphate 4-thio-uridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-tri-phosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deaza-adenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudo-uridine-5′-triphosphate, or puromycin-5′-triphosphate, xanthosine-5′-triphosphate. Particular preference may be given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thiouridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydro-pseudouridine, 2-thio-dihydrouridine, 2-thio-dihydropseudouridine, 2-methoxy-uridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methyl-thio-N6-threonylcarbamoyladenosine, N6,N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-0-(1-thiophosphate)-adenosine, 5′-0-(1-thiophosphate)-cytidine, 5′-0-(1-thiophosphate)-guanosine, 5′-0-(1-thiophosphate)-uridine or 5′-0-(1-thiophosphate)-pseudouridine.

In further embodiments, a modified rRNA may comprise nucleoside modifications selected from 6-aza-cytidine, 2-thio-cytidine, a-thio-cytidine, pseudo-iso-cytidine, 5-aminoallyl-uridine, 5-iodo-uridine, N1-methyl-pseudouridine, 5,6-dihydrouridine, a-thio-uridine, 4-thio-uridine, 6-aza-uridine, 5-hydroxy-uridine, deoxy-thymidine, 5-methyl-uridine, pyrrolo-cytidine, inosine, a-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, a-thio-adenosine, 8-azido-adenosine, 7-deaza-adenosine.

In certain preferred embodiments, the rRNA comprises a modified nucleoside in place of at least one (e.g., every) uridine.

The term “uracil,” as used herein, describes one of the nucleobases that can occur in the nucleic acid of RNA. The structure of uracil is:

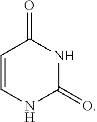

The term “uridine,” as used herein, describes one of the nucleosides that can occur in RNA. The structure of uridine is:

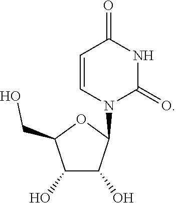 [SMILES: O=c1ccn([C@@H]2O[C@H](CO)[C@@H](O)[C@H]2O)c(=O)[nH]1]

UTP (uridine 5′-triphosphate) has the following structure:

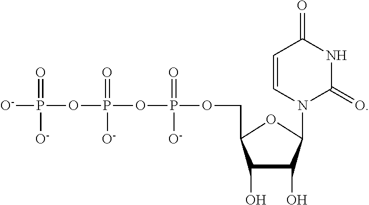

Pseudo-UTP (pseudouridine 5′-triphosphate) has the following structure:

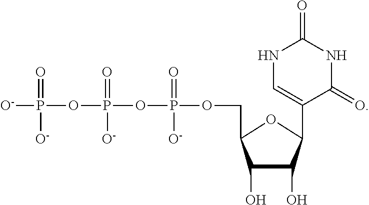

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1Ψ), which has the structure:

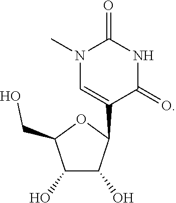 [SMILES: Cn1cc([C@@H]2O[C@H](CO)[C@@H](O)[C@H]2O)c(=O)[nH]c1=O]

N1-methyl-pseudo-UTP has the following structure:

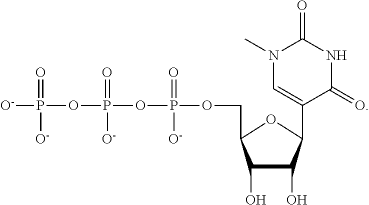

Another exemplary modified nucleoside is 5-methyl-uridine (m5U), which has the structure:

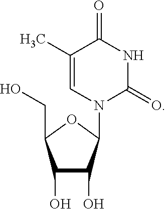

In certain preferred embodiments, one or more uridine in the rRNA described herein is replaced by a modified nucleoside. In some embodiments, the modified nucleoside is a modified uridine.

In certain preferred embodiments, RNA comprises a modified nucleoside in place of at least one uridine. In some embodiments, RNA comprises a modified nucleoside in place of each uridine.

In certain preferred embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In certain preferred embodiments, the modified nucleoside replacing one or more, e.g., all, uridine in the rRNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In an embodiment, the rRNA comprises other modified nucleosides or comprises further modified nucleosides, e.g., modified cytidine such as those described above. For example, in one embodiment, in the rRNA 5-methylcytidine is substituted partially or completely, preferably completely, for cytidine. In one embodiment, the rRNA comprises 5-methylcytidine and one or more selected from pseudouridine (ψ), N1-methyl-pseudouridine (m1ψ), and 5-methyl-uridine (m5U). In an embodiment, the rRNA comprises 5-methylcytidine and N1-methyl-pseudouridine (m1ψ). In some embodiments, the rRNA comprises 5-methylcytidine in place of each cytidine and N1-methyl-pseudouridine (m1ψ) in place of each uridine.

### Functional Non-Structural Protein

The term “non-structural protein” relates to a protein encoded by a virus but that is not part of the viral particle. This term typically includes the various enzymes and transcription factors the virus uses to replicate itself, such as RNA replicase or other template-directed polymerases. The term “non-structural protein” includes each and every co- or post-translationally modified form, including carbohydrate-modified (such as glycosylated) and lipid-modified forms of a non-structural protein and preferably relates to an “alphavirus non-structural protein”.

In some embodiments, the term “alphavirus non-structural protein” refers to any one or more of individual non-structural proteins of alphavirus origin (nsP1, nsP2, nsP3, nsP4), or to a poly-protein comprising the polypeptide sequence of more than one non-structural protein of alphavirus origin.

In some embodiments, “alphavirus non-structural protein” refers to nsP123 and/or to nsP4. In other embodiments, “alphavirus non-structural protein” refers to nsP1234. In one embodiment, the protein of interest encoded by an open reading frame consists of all of nsP1, nsP2, nsP3 and nsP4 as one single, optionally cleavable poly-protein: nsP1234. In one embodiment, the protein of interest encoded by an open reading frame consists of nsP1, nsP2 and nsP3 as one single, optionally cleavable polyprotein: nsP123. In that embodiment, nsP4 may be a further protein of interest and may be encoded by a further open reading frame.

In some embodiments, non-structural protein is capable of forming a complex or association, e.g., in a host cell. In some embodiments, “alphavirus non-structural protein” refers to a complex or association of nsP123 (synonymously P123) and nsP4. In some embodiments, “alphavirus non-structural protein” refers to a complex or association of nsP1, nsP2, and nsP3. In some embodiments, “alphavirus non-structural protein” refers to a complex or association of nsP1, nsP2, nsP3 and nsP4. In some embodiments, “alphavirus non-structural protein” refers to a complex or association of any one or more selected from the group consisting of nsP1, nsP2, nsP3 and nsP4. In some embodiments, the alphavirus non-structural protein comprises at least nsP4.

The terms “complex” or “association” refer to two or more same or different protein molecules that are in spatial proximity. Proteins of a complex are preferably in direct or indirect physical or physicochemical contact with each other. A complex or association can consist of multiple different proteins (heteromultimer) and/or of multiple copies of one particular protein (homomultimer). In the context of alphavirus non-structural protein, the term “complex or association” describes a multitude of at least two protein molecules, of which at least one is an alphavirus non-structural protein. The complex or association can consist of multiple copies of one particular protein (homomultimer) and/or of multiple different proteins (heteromultimer). In the context of a multimer, “multi” means more than one, such as two, three, four, five, six, seven, eight, nine, ten, or more than ten.

The term “functional non-structural protein” includes non-structural protein that has replicase function. Thus, “functional non-structural protein” includes alphavirus replicase. “Replicase function” comprises the function of an RNA-dependent RNA polymerase (RdRP), i.e., an enzyme which is capable to catalyze the synthesis of (−) strand RNA based on a (+) strand RNA template, and/or which is capable to catalyze the synthesis of (+) strand RNA based on a (−) strand RNA template. Thus, the term “functional non-structural protein” can refer to a protein or complex that synthesizes (−) stranded RNA, using the (+) stranded (e.g. genomic) RNA as template, to a protein or complex that synthesizes new (+) stranded RNA, using the (−) stranded complement of genomic RNA as template, and/or to a protein or complex that synthesizes a subgenomic transcript, using a fragment of the (−) stranded complement of genomic RNA as template. The functional non-structural protein may additionally have one or more additional functions, such as, e.g., a protease (for auto-cleavage), helicase, terminal adenylyltransferase (for poly(A) tail addition), methyltransferase and guanylyltransferase (for providing a nucleic acid with a 5′-cap), nuclear localization sites, triphosphatase (Gould et al., 2010, Antiviral Res. 87:111-124; Rupp et al., 2015, J. Gen. Virol. 96:2483-500).

The term “replicase” includes RNA-dependent RNA polymerase. According to the invention, the term “replicase” includes “alphavirus replicase”, including a RNA-dependent RNA polymerase from a naturally occurring alphavirus (alphavirus found in nature) and a RNA-dependent RNA polymerase from a variant or derivative of an alphavirus, such as from an attenuated alphavirus.

The term “replicase” comprises all variants, in particular post-translationally modified variants, conformations, isoforms and homologs of alphavirus replicase, which are expressed by alphavirus-infected cells or which are expressed by cells that have been transfected with a nucleic acid that codes for alphavirus replicase. Moreover, the term “replicase” comprises all forms of replicase that have been produced and can be produced by recombinant methods. For example, a replicase comprising a tag that facilitates detection and/or purification of the replicase in the laboratory, e.g.; a myc-tag, a HA-tag or an oligohistidine tag (His-tag) may be produced by recombinant methods.

Optionally, the alphavirus replicase is additionally functionally defined by the capacity of binding to any one or more of alphavirus conserved sequence element 1 (CSE 1) or complementary sequence thereof, conserved sequence element 2 (CSE 2) or complementary sequence thereof, conserved sequence element 3 (CSE 3) or complementary sequence thereof, conserved sequence element 4 (CSE 4) or complementary sequence thereof. Preferably, the replicase is capable of binding to CSE 2 [i.e., to the (+) strand] and/or to CSE 4 [i.e., to the (+) strand], or of binding to the complement of CSE 1 [i.e. to the (−) strand] and/or to the complement of CSE 3 [i.e., to the (−) strand].

The origin of the alphavirus replicase is not limited to any particular alphavirus. In a preferred embodiment, the alphavirus replicase comprises non-structural protein from Semliki Forest virus, including a naturally occurring Semliki Forest virus and a variant or derivative of Semliki Forest virus, such as an attenuated Semliki Forest virus. In an alternative preferred embodiment, the alphavirus replicase comprises non-structural protein from Sindbis virus, including a naturally occurring Sindbis virus and a variant or derivative of Sindbis virus, such as an attenuated Sindbis virus. In an alternative preferred embodiment, the alphavirus replicase comprises non-structural protein from Venezuelan equine encephalitis virus (VEEV), including a naturally occurring VEEV and a variant or derivative of VEEV, such as an attenuated VEEV. In an alternative preferred embodiment, the alphavirus replicase comprises non-structural protein from chikungunya virus (CHIKV), including a naturally occurring CHIKV and a variant or derivative of CHIKV, such as an attenuated CHIKV.

A replicase can also comprise non-structural proteins from more than one virus, e.g., from more than one alphavirus. Thus, heterologous complexes or associations comprising alphavirus non-structural protein and having replicase function are equally comprised by the present invention. Merely for illustrative purposes, replicase may comprise one or more non-structural proteins (e.g., nsP1, nsP2) from a first alphavirus, and one or more non-structural proteins (nsP3, nsP4) from a second alphavirus. Non-structural proteins from more than one different alphavirus may be encoded by separate open reading frames, or may be encoded by a single open reading frame as poly-protein, e.g., nsP1234.

In some embodiments, functional non-structural protein is capable of forming membranous replication complexes and/or vacuoles in cells in which the functional non-structural protein is expressed.

If functional non-structural protein, i.e., non-structural protein with replicase function, is encoded by a nucleic acid molecule according to the present invention, it is preferable that the subgenomic promoter of the replicon, if present, is compatible with said replicase. Compatible in this context means that the replicase is capable of recognizing the subgenomic promoter, if present. In one embodiment, this is achieved when the subgenomic promoter is native to the virus from which the replicase is derived, i.e. the natural origin of these sequences is the same virus. In an alternative embodiment, the subgenomic promoter is not native to the virus from which the virus replicase is derived, provided that the virus replicase is capable of recognizing the subgenomic promoter. In other words, the replicase is compatible with the subgenomic promoter (cross-virus compatibility). Examples of cross-virus compatibility concerning subgenomic promoter and replicase originating from different alphaviruses are known in the art. Any combination of subgenomic promoter and replicase is possible as long as cross-virus compatibility exists. Cross-virus compatibility can readily be tested by the skilled person working the present invention by incubating a replicase to be tested together with an RNA, wherein the RNA has a subgenomic promoter to be tested, at conditions suitable for RNA synthesis from the a subgenomic promoter. If a subgenomic transcript is prepared, the subgenomic promoter and the replicase are determined to be compatible. Various examples of cross-virus compatibility are known.

The replicon can preferably be replicated by the functional non-structural protein. In particular, the RNA replicon that encodes functional non-structural protein can be replicated by the functional non-structural protein encoded by the replicon. In a preferred embodiment, the RNA replicon comprises an open reading frame encoding functional alphavirus non-structural protein. In one embodiment, the replicon comprises a further open reading frame encoding a protein of interest. This embodiment is particularly suitable in some methods for producing a protein of interest according to the present invention. In one embodiment, the further open reading frame encoding a protein of interest is located downstream from the 5′ replication recognition sequence and upstream from the IRES (and upstream from the open reading frame encoding a functional non-structural protein from a self-replicating virus) and/or downstream from the open reading frame encoding a functional non-structural protein from a self-replicating virus. The further open reading frame encoding a protein of interest located downstream from the 5′ replication recognition sequence and upstream from the IRES (and upstream from the open reading frame encoding a functional non-structural protein from a self-replicating virus) may be expressed as a fusion protein with sequences encoded by the 5′ replication recognition sequence. The further open reading frame encoding a protein of interest located downstream from the 5′ replication recognition sequence and upstream from the IRES (and upstream from the open reading frame encoding a functional non-structural protein from a self-replicating virus) may be or may not be controlled by a subgenomic promoter. The one or more further open reading frames encoding one or more proteins of interest located downstream from the open reading frame encoding a functional non-structural protein from a self-replicating virus are generally controlled by (a) subgenomic promoter(s).

It is preferable that the open reading frame encoding functional non-structural protein does not overlap with the 5′ replication recognition sequence. In one embodiment, the open reading frame encoding functional non-structural protein does not overlap with the subgenomic promoter, if present. Embodiments thereof are disclosed in WO 2017/162460, herein incorporated by reference.

### Uncoupling of Sequence Elements Required for Replication and Protein-Coding Regions

Versatile alphavirus-derived vectors are difficult to develop because the open reading frame encoding nsP1234 overlaps with the 5′ replication recognition sequence of the alphavirus genome (coding sequence for nsP1) and typically also with the subgenomic promoter comprising CSE 3 (coding sequence for nsP4).

The RNA replicon described herein generally comprises sequence elements required for replication by the replicase, in particular a 5′ replication recognition sequence. In an embodiment, the coding sequence for non-structural protein is under the control of an IRES and thus an IRES is located upstream of the coding sequence for non-structural protein. Thus, in one embodiment, the 5′ replication recognition sequence which normally overlaps with the coding sequence for the N-terminal fragment of the alphavirus non-structural protein, is located upstream of the IRES and does not overlap with the coding sequence for non-structural protein.

In an embodiment, coding sequences of the 5′ replication recognition sequence such as nsP1 coding sequences are fused in frame to a gene of interest which is located upstream from the IRES.

In an embodiment, the 5′ replication recognition sequence does not encode any protein or fragment thereof, such as an alphavirus non-structural protein or fragment thereof. Thus, in the RNA replicon according to the invention, the sequence elements required for replication by the replicase and protein-coding regions may be uncoupled. The uncoupling may be achieved by the removal of at least one initiation codon in the 5′ replication recognition sequence compared to a native virus genomic RNA, e.g., native alphavirus genomic RNA.

Thus, the rRNA may comprise a 5′ replication recognition sequence, wherein the 5′ replication recognition sequence is characterized in that it comprises the removal of at least one initiation codon compared to a native virus 5′ replication recognition sequence, e.g., native alphavirus 5′ replication recognition sequence.

The 5′ replication recognition sequence that is characterized in that it comprises the removal of at least one initiation codon compared to a native virus 5′ replication recognition sequence can be referred to herein as “modified 5′ replication recognition sequence” or “5′ replication recognition sequence according to the invention”. As described herein below, the 5′ replication recognition sequence according to the invention may optionally be characterized by the presence of one or more additional nucleotide changes, such as those detected by the methods of the present invention.

A nucleic acid construct that is capable of being replicated by a replicase, preferably an alphaviral replicase, is termed replicable RNA or replicon. According to the invention, the term “replicon” defines an RNA molecule that can be replicated by RNA-dependent RNA polymerase, yielding—without DNA intermediate—one or multiple identical or essentially identical copies of the RNA replicon. “Without DNA intermediate” means that no deoxyribonucleic acid (DNA) copy or complement of the replicon is formed in the process of forming the copies of the RNA replicon, and/or that no deoxyribonucleic acid (DNA) molecule is used as a template in the process of forming the copies of the RNA replicon, or complement thereof. The replicase function is typically provided by functional non-structural protein, e.g., functional alphavirus non-structural protein.

According to the invention, the terms “can be replicated” and “capable of being replicated” generally describe that one or more identical or essentially identical copies of a nucleic acid can be prepared. When used together with the term “replicase”, such as in “capable of being replicated by a replicase”, the terms “can be replicated” and “capable of being replicated” describe functional characteristics of a nucleic acid molecule, e.g. a RNA replicon, with respect to a replicase. These functional characteristics comprise at least one of (i) the replicase is capable of recognizing the replicon and (ii) the replicase is capable of acting as RNA-dependent RNA polymerase (RdRP). Preferably, the replicase is capable of both (i) recognizing the replicon and (ii) acting as RNA-dependent RNA polymerase.

The expression “capable of recognizing” describes that the replicase is capable of physically associating with the replicon, and preferably, that the replicase is capable of binding to the replicon, typically non-covalently. The term “binding” can mean that the replicase has the capacity of binding to any one or more of a conserved sequence element 1 (CSE 1) or complementary sequence thereof (if comprised by the replicon), conserved sequence element 2 (CSE 2) or complementary sequence thereof (if comprised by the replicon), conserved sequence element 3 (CSE 3) or complementary sequence thereof (if comprised by the replicon), conserved sequence element 4 (CSE 4) or complementary sequence thereof (if comprised by the replicon). Preferably, the replicase is capable of binding to CSE 2 [i.e., to the (+) strand] and/or to CSE 4 [i.e., to the (+) strand], or of binding to the complement of CSE 1 [i.e. to the (−) strand] and/or to the complement of CSE 3 [i.e., to the (−) strand].

In one embodiment, the expression “capable of acting as RdRP” means that the replicase is capable to catalyze the synthesis of the (−) strand complement of viral genomic (+) strand RNA, wherein the (+) strand RNA has template function, and/or that the replicase is capable to catalyze the synthesis of (+) strand viral genomic RNA, wherein the (−) strand RNA has template function. In general, the expression “capable of acting as RdRP” can also include that the replicase is capable to catalyze the synthesis of a (+) strand subgenomic transcript wherein a (−) strand RNA has template function, and wherein synthesis of the (+) strand subgenomic transcript is typically initiated at a subgenomic promoter. In one embodiment, the virus is an alphavirus.

The expressions “capable of binding” and “capable of acting as RdRP” refer to the capability at normal physiological conditions. In particular, they refer to the conditions inside a cell, which expresses functional non-structural protein or which has been transfected with a nucleic acid that codes for functional non-structural protein. The cell is preferably a eukaryotic cell. The capability of binding and/or the capability of acting as RdRP can be experimentally tested, e.g. in a cell-free in vitro system or in a eukaryotic cell. Optionally, said eukaryotic cell is a cell from a species to which the particular virus that represents the origin of the replicase is infectious. For example, when the virus replicase from a particular virus is used that is infectious to humans, the normal physiological conditions are conditions in a human cell. More preferably, the eukaryotic cell (in one example human cell) is from the same tissue or organ to which the particular virus that represents the origin of the replicase is infectious.

According to the invention, “compared to a native alphavirus sequence” and similar terms refer to a sequence that is a variant of a native alphavirus sequence. The variant is typically not itself a native alphavirus sequence.

In one embodiment, the RNA replicon comprises a 3′ replication recognition sequence. A 3′ replication recognition sequence is a nucleic acid sequence that can be recognized by functional non-structural protein. In other words, functional non-structural protein is capable of recognizing the 3′ replication recognition sequence. Preferably, the 3′ replication recognition sequence is located at the 3′ end of the replicon (if the replicon does not comprise a poly(A) tail), or immediately upstream of the poly(A) tail (if the replicon comprises a poly(A) tail). In one embodiment, the 3′ replication recognition sequence consists of or comprises CSE 4.

In one embodiment, the 5′ replication recognition sequence and the 3′ replication recognition sequence are capable of directing replication of the RNA replicon according to the present invention in the presence of functional non-structural protein. Thus, when present alone or preferably together, these recognition sequences direct replication of the RNA replicon in the presence of functional non-structural protein.

It is preferable that a functional non-structural protein is provided that is capable of recognizing both the 5′ replication recognition sequence and the 3′ replication recognition sequence of the replicon. In one embodiment, this is achieved when the 3′ replication recognition sequence is native to the alphavirus from which the functional alphavirus non-structural protein is derived, and when the 5′ replication recognition sequence is native to the alphavirus from which the functional alphavirus non-structural protein is derived or is a variant of the 5′ replication recognition sequence that is native to the alphavirus from which the functional alphavirus non-structural protein is derived. Native means that the natural origin of these sequences is the same alphavirus. In an alternative embodiment, the 5′ replication recognition sequence and/or the 3′ replication recognition sequence are not native to the alphavirus from which the functional alphavirus non-structural protein is derived, provided that the functional alphavirus non-structural protein is capable of recognizing both the 5′ replication recognition sequence and the 3′ replication recognition sequence of the replicon. In other words, the functional alphavirus non-structural protein is compatible to the 5′ replication recognition sequence and the 3′ replication recognition sequence. When a non-native functional alphavirus non-structural protein is capable of recognizing a respective sequence or sequence element, the functional alphavirus non-structural protein is said to be compatible (cross-virus compatibility). Any combination of (3′/5′) replication recognition sequences and CSEs, respectively, with functional alphavirus non-structural protein is possible as long as cross-virus compatibility exists. Cross-virus compatibility can readily be tested by the skilled person working the present invention by incubating a functional alphavirus non-structural protein to be tested together with an RNA, wherein the RNA has 3′- and 5′ replication recognition sequences to be tested, at conditions suitable for RNA replication, e.g. in a suitable host cell. If replication occurs, the (3′/5′) replication recognition sequences and the functional alphavirus non-structural protein are determined to be compatible.

The removal of at least one initiation codon within the 5′ replication recognition sequence provides several advantages. Absence of an initiation codon in the nucleic acid sequence encoding nsP1* (N-terminal fragment of nsP1) will typically cause that nsP1* is not translated. Further, since nsP1* is not translated, the open reading frame encoding the protein of interest (“GOI 2”) is the most upstream open reading frame accessible to the ribosome; thus, when the replicon is present in a cell, translation is initiated at the first AUG of the open reading frame (RNA) encoding the gene of interest.

The removal of at least one initiation codon can be achieved by any suitable method known in the art. For example, a suitable DNA molecule encoding the replicon according to the invention, i.e., characterized by the removal of an initiation codon, can be designed in silico, and subsequently synthesized in vitro (gene synthesis); alternatively, a suitable DNA molecule may be obtained by site-directed mutagenesis of a DNA sequence encoding a replicon. In any case, the respective DNA molecule may serve as template for in vitro transcription, thereby providing the replicon according to the invention.

The removal of at least one initiation codon compared to a native 5′ replication recognition sequence is not particularly limited and may be selected from any nucleotide modification, including substitution of one or more nucleotides (including, on DNA level, a substitution of A and/or T and/or G of the initiation codon); deletion of one or more nucleotides (including, on DNA level, a deletion of A and/or T and/or G of the initiation codon), and insertion of one or more nucleotides (including, on DNA level, an insertion of one or more nucleotides between A and T and/or between T and G of the initiation codon). Irrespective of whether the nucleotide modification is a substitution, an insertion or a deletion, the nucleotide modification must not result in the formation of a new initiation codon (as an illustrative example: an insertion, at DNA level, must not be an insertion of an ATG).

The 5′ replication recognition sequence of the RNA replicon that is characterized by the removal of at least one initiation codon (i.e. the modified 5′ replication recognition sequence according to the present invention) is preferably a variant of a 5′ replication recognition sequence of the genome of an alphavirus found in nature. In one embodiment, the modified 5′ replication recognition sequence according to the present invention is preferably characterized by a degree of sequence identity of 80% or more, preferably 85% or more, more preferably 90% or more, even more preferably 95% or more, to the 5′ replication recognition sequence of the genome of at least one alphavirus found in nature.

In one embodiment, the 5′ replication recognition sequence of the RNA replicon that may be characterized by the removal of at least one initiation codon comprises a sequence homologous to about 250 nucleotides at the 5′ end of an alphavirus, i.e. at the 5′ end of the alphaviral genome. In a preferred embodiment, it comprises a sequence homologous to about 250 to 500, preferably about 300 to 500 nucleotides at the 5′ end of an alphavirus, i.e., at the 5′ end of the alphaviral genome. “At the 5′ end of the alphaviral genome” means a nucleic acid sequence beginning at, and including, the most upstream nucleotide of the alphaviral genome. In other words, the most upstream nucleotide of the alphaviral genome is designated nucleotide no. 1, and, e.g., “250 nucleotides at the 5′ end of the alphaviral genome” means nucleotides 1 to 250 of the alphaviral genome. In one embodiment, the 5′ replication recognition sequence of the RNA replicon is characterized by a degree of sequence identity of 80% or more, preferably 85% or more, more preferably 90% or more, even more preferably 95% or more, to at least 250 nucleotides at the 5′ end of the genome of at least one alphavirus found in nature. At least 250 nucleotides includes, e.g., 250 nucleotides, 300 nucleotides, 400 nucleotides, 500 nucleotides.

The 5′ replication recognition sequence of an alphavirus found in nature is typically characterized by at least one initiation codon and/or by conserved secondary structural motifs. For example, the native 5′ replication recognition sequence of Semliki Forest virus (SFV) comprises five specific AUG base triplets. According to Frolov et al., 2001, RNA 7:1638-1651, analysis by MFOLD revealed that the native 5′ replication recognition sequence of Semliki Forest virus is predicted to form four stem loops (SL), termed stem loops 1 to 4 (SL1, SL2, SL3, SL4). According to Frolov et al., analysis by MFOLD revealed that also the native 5′ replication recognition sequence of a different alphavirus, Sindbis virus, is predicted to form four stem loops: SL1, SL2, SL3, SL4.

It is known that the 5′ end of the alphaviral genome comprises sequence elements that enable replication of the alphaviral genome by functional alphavirus non-structural protein. In one embodiment of the present invention, the 5′ replication recognition sequence of the RNA replicon comprises a sequence homologous to conserved sequence element 1 (CSE 1) and/or a sequence homologous to conserved sequence element 2 (CSE 2) of an alphavirus.

Conserved sequence element 2 (CSE 2) of alphavirus genomic RNA typically is represented by SL3 and SL4 which is preceded by SL2 comprising at least the native initiation codon that encodes the first amino acid residue of alphavirus non-structural protein nsP1. In this description, however, in some embodiments, the conserved sequence element 2 (CSE 2) of alphavirus genomic RNA refers to a region spanning from SL2 to SL4 and comprising the native initiation codon that encodes the first amino acid residue of alphavirus non-structural protein nsP1. In a preferred embodiment, the RNA replicon comprises CSE 2 or a sequence homologous to CSE 2. In one embodiment, the RNA replicon comprises a sequence homologous to CSE 2 that is preferably characterized by a degree of sequence identity of 80% or more, preferably 85% or more, more preferably 90% or more, even more preferably 95% or more, to the sequence of CSE 2 of at least one alphavirus found in nature.

In an embodiment, the 5′ replication recognition sequence comprises a sequence that is homologous to CSE 2 of an alphavirus. The CSE 2 of an alphavirus may comprise a fragment of an open reading frame of a non-structural protein from an alphavirus.

Thus, in an embodiment, the RNA replicon is characterized in that it comprises a sequence homologous to an open reading frame of a non-structural protein or a fragment thereof from an alphavirus. The sequence homologous to an open reading frame of a non-structural protein or a fragment thereof is typically a variant of an open reading frame of a non-structural protein or a fragment thereof of an alphavirus found in nature. In one embodiment, the sequence homologous to an open reading frame of a non-structural protein or a fragment thereof is preferably characterized by a degree of sequence identity of 80% or more, preferably 85% or more, more preferably 90% or more, even more preferably 95% or more, to an open reading frame of a non-structural protein or a fragment thereof of at least one alphavirus found in nature.

In an embodiment, the sequence homologous to an open reading frame of a non-structural protein that is comprised by the replicon of the present invention does not comprise the native initiation codon of a non-structural protein, and more preferably does not comprise any initiation codon of a non-structural protein. In an embodiment, the sequence homologous to CSE 2 is characterized by the removal of all initiation codons compared to a native alphavirus CSE 2 sequence. Thus, the sequence homologous to CSE 2 does preferably not comprise any initiation codon.

When the sequence homologous to an open reading frame does not comprise any initiation codon, the sequence homologous to an open reading frame is not itself an open reading frame since it does not serve as a template for translation.

In one embodiment, the 5′ replication recognition sequence comprises a sequence homologous to an open reading frame of a non-structural protein or a fragment thereof from an alphavirus, wherein the sequence homologous to an open reading frame of a non-structural protein or a fragment thereof from an alphavirus is characterized in that it comprises the removal of at least one initiation codon compared to the native alphavirus sequence.

In an embodiment, the sequence homologous to an open reading frame of a non-structural protein or a fragment thereof from an alphavirus is characterized in that it comprises the removal of at least the native start codon of the open reading frame of a non-structural protein. Preferably, it is characterized in that it comprises the removal of at least the native start codon of the open reading frame encoding nsP1.

The native start codon is the AUG base triplet at which translation on ribosomes in a host cell begins when an RNA is present in a host cell. In other words, the native start codon is the first base triplet that is translated during ribosomal protein synthesis, e.g., in a host cell that has been inoculated with RNA comprising the native start codon. In one embodiment, the host cell is a cell from a eukaryotic species that is a natural host of the specific alphavirus that comprises the native alphavirus 5′ replication recognition sequence. In an embodiment, the host cell is a BHK21 cell from the cell line “BHK21 [C13](ATCC® CCL10™)”, available from American Type Culture Collection, Manassas, Virginia, USA.

The genomes of many alphaviruses have been fully sequenced and are publicly accessible, and the sequences of non-structural proteins encoded by these genomes are publicly accessible as well. Such sequence information allows to determine the native start codon in silico.

In an embodiment, the sequence homologous to an open reading frame of a non-structural protein or a fragment thereof from an alphavirus is characterized in that it comprises the removal of one or more initiation codons other than the native start codon of the open reading frame of a non-structural protein. In an embodiment, said nucleic acid sequence is additionally characterized by the removal of the native start codon. For example, in addition to the removal of the native start codon, any one or two or three or four or more than four (e.g., five) initiation codons may be removed.

If the replicon is characterized by the removal of the native start codon, and optionally by the removal of one or more initiation codons other than the native start codon, of the open reading frame of a non-structural protein, the sequence homologous to an open reading frame is not itself an open reading frame since it does not serve as a template for translation.

The one or more initiation codon other than the native start codon that is removed, preferably in addition to removal of the native start codon, is preferably selected from an AUG base triplet that has the potential to initiate translation. An AUG base triplet that has the potential to initiate translation may be referred to as “potential initiation codon”. Whether a given AUG base triplet has the potential to initiate translation can be determined in silico or in a cell-based in vitro assay.

In one embodiment, it is determined in silico whether a given AUG base triplet has the potential to initiate translation: in that embodiment, the nucleotide sequence is examined, and an AUG base triplet is determined to have the potential to initiate translation if it is part of an AUGG sequence, preferably part of a Kozak sequence.

In one embodiment, it is determined in a cell-based in vitro assay whether a given AUG base triplet has the potential to initiate translation: an RNA replicon characterized by the removal of the native start codon and comprising the given AUG base triplet downstream of the position of the removal of the native start codon is introduced into a host cell. In one embodiment, the host cell is a cell from a eukaryotic species that is a natural host of the specific alphavirus that comprises the native alphavirus 5′ replication recognition sequence. In a preferred embodiment, the host cell is a BHK21 cell from the cell line “BHK21 [C13](ATCC® CCL10™)”, available from American Type Culture Collection, Manassas, Virginia, USA. It is preferable that no further AUG base triplet is present between the position of the removal of the native start codon and the given AUG base triplet. If, following transfer of the RNA replicon—characterized by the removal of the native start codon and comprising the given AUG base triplet—into the host cell, translation is initiated at the given AUG base triplet, the given AUG base triplet is determined to have the potential to initiate translation. Whether translation is initiated can be determined by any suitable method known in the art. For example, the replicon may encode, downstream of the given AUG base triplet and in-frame with the given AUG base triplet, a tag that facilitates detection of the translation product (if any), e.g. a myc-tag or a HA-tag; whether or not an expression product having the encoded tag is present may be determined e.g. by Western Blot. In this embodiment, it is preferable that no further AUG base triplet is present between the given AUG base triplet and the nucleic acid sequence encoding the tag. The cell-based in vitro assay can be performed individually for more than one given AUG base triplet: in each case, it is preferable that no further AUG base triplet is present between the position of the removal of the native start codon and the given AUG base triplet. This can be achieved by removing all AUG base triplets (if any) between the position of the removal of the native start codon and the given AUG base triplet. Thereby, the given AUG base triplet is the first AUG base triplet downstream of the position of the removal of the native start codon.

Preferably, the 5′ replication recognition sequence of the RNA replicon according to the present invention is characterized by the removal of all potential initiation codons. Thus, according to the invention, the 5′ replication recognition sequence preferably does not comprise an open reading frame that can be translated to protein.

In an embodiment, the 5′ replication recognition sequence of the RNA replicon according to the invention is characterized by a secondary structure that is equivalent to the (predicted) secondary structure of the 5′ replication recognition sequence of viral genomic RNA. To this end, the RNA replicon may comprise one or more nucleotide changes compensating for nucleotide pairing disruptions within one or more stem loops introduced by the removal of at least one initiation codon.

In an embodiment, the 5′ replication recognition sequence of the RNA replicon according to the invention is characterized by a secondary structure that is equivalent to the secondary structure of the 5′ replication recognition sequence of alphaviral genomic RNA. In a preferred embodiment, the 5′ replication recognition sequence of the RNA replicon according to the invention is characterized by a predicted secondary structure that is equivalent to the predicted secondary structure of the 5′ replication recognition sequence of alphaviral genomic RNA. According to the present invention, the secondary structure of an RNA molecule is preferably predicted by the web server for RNA secondary structure prediction http://rna.urmc.rochester.edu/RNAstructureWeb/Servers/Predict1/Predict1.html.

By comparing the secondary structure or predicted secondary structure of a 5′ replication recognition sequence of an RNA replicon characterized by the removal of at least one initiation codon compared to the native alphavirus 5′ replication recognition sequence, the presence or absence of a nucleotide pairing disruption can be identified. For example, at least one base pair may be absent at a given position, compared to a native alphavirus 5′ replication recognition sequence, e.g. a base pair within a stem loop, in particular the stem of the stem loop.

In an embodiment, one or more stem loops of the 5′ replication recognition sequence are not deleted or disrupted. More preferably, stem loops 3 and 4 are not deleted or disrupted. Preferably, none of the stem loops of the 5′ replication recognition sequence is deleted or disrupted.

In one embodiment, the removal of at least one initiation codon does not disrupt the secondary structure of the 5′ replication recognition sequence. In an alternative embodiment, the removal of at least one initiation codon does disrupt the secondary structure of the 5′ replication recognition sequence. In this embodiment, the removal of at least one initiation codon may be causative for the absence of at least one base pair at a given position, e.g. a base pair within a stem loop, compared to a native 5′ replication recognition sequence. If a base pair is absent within a stem loop, compared to a native 5′ replication recognition sequence, the removal of at least one initiation codon is determined to introduce a nucleotide pairing disruption within the stem loop. A base pair within a stem loop is typically a base pair in the stem of the stem loop.

In an embodiment, the RNA replicon comprises one or more nucleotide changes compensating for nucleotide pairing disruptions within one or more stem loops introduced by the removal of at least one initiation codon.

If the removal of at least one initiation codon introduces a nucleotide pairing disruption within a stem loop, compared to a native 5′ replication recognition sequence, one or more nucleotide changes may be introduced which are expected to compensate for the nucleotide pairing disruption, and the secondary structure or predicted secondary structure obtained thereby may be compared to a native 5′ replication recognition sequence.

Based on the common general knowledge and on the disclosure herein, certain nucleotide changes can be expected by the skilled person to compensate for nucleotide pairing disruptions. For example, if a base pair is disrupted at a given position of the secondary structure or predicted secondary structure of a given 5′ replication recognition sequence of an RNA replicon characterized by the removal of at least one initiation codon, compared to the native 5′ replication recognition sequence, a nucleotide change that restores a base pair at that position, preferably without re-introducing an initiation codon, is expected to compensate for the nucleotide pairing disruption.

In an embodiment, the 5′ replication recognition sequence of the replicon does not overlap with, or does not comprise, a translatable nucleic acid sequence, i.e. translatable into a peptide or protein, in particular a nsP, in particular nsP1, or a fragment of any thereof. For a nucleotide sequence to be “translatable”, it requires the presence of an initiation codon; the initiation codon encodes the most N-terminal amino acid residue of the peptide or protein. In one embodiment, the 5′ replication recognition sequence of the replicon does not overlap with, or does not comprise, a translatable nucleic acid sequence encoding an N-terminal fragment of nsP1.

In some scenarios, the RNA replicon comprises at least one subgenomic promoter. In a preferred embodiment, the subgenomic promoter of the replicon does not overlap with, or does not comprise, a translatable nucleic acid sequence, i.e. translatable into a peptide or protein, in particular a nsP, in particular nsP4, or a fragment of any thereof. In one embodiment, the subgenomic promoter of the replicon does not overlap with, or does not comprise, a translatable nucleic acid sequence that encodes a C-terminal fragment of nsP4. A RNA replicon having a subgenomic promoter that does not overlap with, or does not comprise, a translatable nucleic acid sequence, e.g. translatable into the C-terminal fragment of nsP4, may be generated by deleting part of the coding sequence for nsP4 (typically the part encoding the N-terminal part of nsP4), and/or by removing AUG base triplets in the part of the coding sequence for nsP4 that has not been deleted. If AUG base triplets in the coding sequence for nsP4 or a part thereof are removed, the AUG base triplets that are removed are preferably potential initiation codons. Alternatively, if the subgenomic promoter does not overlap with a nucleic acid sequence that encodes nsP4, the entire nucleic acid sequence encoding nsP4 may be deleted.

In an embodiment, the RNA replicon does not comprise an open reading frame encoding a truncated non-structural protein, e.g., a truncated alphavirus non-structural protein. In the context of this embodiment, it is particularly preferable that the RNA replicon does not comprise an open reading frame encoding the N-terminal fragment of nsP1, and optionally does not comprise an open reading frame encoding the C-terminal fragment of nsP4. The N-terminal fragment of nsP1 is a truncated alphavirus protein; the C-terminal fragment of nsP4 is also a truncated alphavirus protein.

In some embodiments, the replicon according to the present invention does not comprise stem loop 2 (SL2) of the 5′ terminus of the genome of an alphavirus. According to Frolov et al., supra, stem loop 2 is a conserved secondary structure found at the 5′ terminus of the genome of an alphavirus, upstream of CSE 2, but is dispensable for replication.

The RNA replicon according to the present invention is preferably a single stranded RNA molecule. The RNA replicon according to the present invention is typically a (+) stranded RNA molecule. In one embodiment, the RNA replicon of the present invention is an isolated nucleic acid molecule. The RNA replicon according to the present invention comprises at least one modified nucleotide, and preferably comprises one or more sequence changes, in particular those detected by the methods disclosed herein for identifying sequence changes that restore or improve the function of an rRNA comprising at least one modified nucleotide.

### At Least One Open Reading Frame Encoding at Least One Gene Product of Interest

In one embodiment, the RNA replicon according to the present invention comprises at least one open reading frame encoding a gene product of interest, such as a peptide of interest or a protein of interest. Preferably, the protein of interest is encoded by a heterologous nucleic acid sequence. The gene encoding the peptide or protein of interest is synonymously termed “gene of interest” or “transgene”. In various embodiments, the peptide or protein of interest is encoded by a heterologous nucleic acid sequence. According to the present invention, the term “heterologous” refers to the fact that a nucleic acid sequence is not naturally functionally or structurally linked to a virus nucleic acid sequence, e.g., an alphavirus nucleic acid sequence.

The replicon according to the present invention may encode a single polypeptide or multiple polypeptides. Multiple polypeptides can be encoded as a single polypeptide (fusion polypeptide) or as separate polypeptides. In some embodiments, the replicon according to the present invention may comprise more than one open reading frames, each of which may independently be selected to be under the control of a subgenomic promoter or not. Alternatively, a poly-protein or fusion polypeptide comprises individual polypeptides separated by a 2A self-cleaving peptides (e.g. from foot-and-mouth disease virus 2A protein), or protease cleavage site or an intein.

Proteins of interest may e.g. be selected from the group consisting of reporter proteins, pharmaceutically active peptides or proteins, inhibitors of intracellular interferon (IFN) signaling.

According to the invention, a protein of interest preferably does not include functional non-structural protein from a self-replicating virus, e.g., functional alphavirus non-structural protein.

### Reporter Protein

In one embodiment, an open reading frame encodes a reporter protein, e.g., a cell-surface expressed protein such as CD90. In that embodiment, the open reading frame comprises a reporter gene. Certain genes may be chosen as reporters because the characteristics they confer on cells or organisms expressing them may be readily identified and measured, or because they are selectable markers. Reporter genes are often used as an indication of whether a certain gene has been taken up by or expressed in the cell or organism population. Preferably, the expression product of the reporter gene is visually detectable. Common visually detectable reporter proteins typically possess fluorescent or luminescent proteins. Examples of specific reporter genes include the gene that encodes jellyfish green fluorescent protein (GFP), which causes cells that express it to glow green under blue light, the enzyme luciferase, which catalyzes a reaction with luciferin to produce light, and the red fluorescent protein (RFP). Variants of any of these specific reporter genes are possible, as long as the variants possess visually detectable properties. For example, eGFP is a point mutant variant of GFP. The reporter protein embodiment is particularly suitable for testing expression.

### Pharmaceutically Active Gene Product, Such as a Peptide or Protein or Nucleic Acid

According to the invention, in one embodiment, rRNA comprises or consists of pharmaceutically active rRNA. A “pharmaceutically active RNA” may be RNA that encodes a pharmaceutically active peptide or protein. Preferably, the RNA replicon according to the present invention encodes a pharmaceutically active peptide or protein or other gene product. Preferably, an open reading frame encodes a pharmaceutically active peptide or protein. Preferably, the RNA replicon comprises an open reading frame that encodes a pharmaceutically active peptide or protein, optionally under control of the subgenomic promoter.

A “pharmaceutically active peptide or protein” has a positive or advantageous effect on the condition or disease state of a subject when administered to the subject in a therapeutically effective amount. Preferably, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or to lessen the severity of such disease or pathological condition. The term “pharmaceutically active peptide or protein” includes entire proteins or polypeptides, and can also refer to pharmaceutically active fragments thereof. It can also include pharmaceutically active analogs of a peptide or protein. The term “pharmaceutically active peptide or protein” includes peptides and proteins that are antigens, i.e., the peptide or protein elicits an immune response in a subject which may be therapeutic or partially or fully protective.

In one embodiment, the pharmaceutically active peptide or protein is or comprises an immunologically active compound or an antigen or an epitope.

According to the invention, the term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. In one embodiment, the immune response involves stimulation of an antibody response (usually including immunoglobulin G (IgG)). Immunologically active compounds possess potent immunostimulating activity including, but not limited to, antiviral and antitumor activity, and can also down-regulate other aspects of the immune response, for example shifting the immune response away from a Th2 immune response, which is useful for treating a wide range of Th2 mediated diseases.

According to the invention, the term “antigen” or “immunogen” covers any substance that will elicit an immune response. In particular, an “antigen” relates to any substance that reacts specifically with antibodies or T-lymphocytes (T-cells). According to the present invention, the term “antigen” comprises any molecule which comprises at least one epitope. Preferably, an antigen in the context of the present invention is a molecule which, optionally after processing, induces an immune reaction, which is preferably specific for the antigen. According to the present invention, any suitable antigen may be used, which is a candidate for an immune reaction, wherein the immune reaction may be both a humoral as well as a cellular immune reaction. In the context of the embodiments of the present invention, the antigen is preferably presented by a cell, preferably by an antigen presenting cell, in the context of MHC molecules, which results in an immune reaction against the antigen. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present invention, an antigen may correspond to a naturally occurring product, for example, a viral protein, or a part thereof. In preferred embodiments, the antigen is a surface polypeptide, i.e. a polypeptide naturally displayed on the surface of a cell, a pathogen, a bacterium, a virus, a fungus, a parasite, an allergen, or a tumor. The antigen may elicit an immune response against a cell, a pathogen, a bacterium, a virus, a fungus, a parasite, an allergen, or a tumor.

The term “pathogen” refers to pathogenic biological material capable of causing disease in an organism, preferably a vertebrate organism. Pathogens include microorganisms such as bacteria, unicellular eukaryotic organisms (protozoa), fungi, as well as viruses.

The terms “epitope”, “antigen peptide”, “antigen epitope”, “immunogenic peptide” and “MHC binding peptide” are used interchangeably herein and refer to an antigenic determinant in a molecule such as an antigen, i.e., to a part in or fragment of an immunologically active compound that is recognized by the immune system, for example, that is recognized by a T cell, in particular when presented in the context of MHC molecules. An epitope of a protein preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 9, 10, 11, 12, 13, 14, 15, 16, 17, 18, 19, 20, 21, 22, 23, 24, or 25 amino acids in length. According to the invention an epitope may bind to MHC molecules such as MHC molecules on the surface of a cell and thus, may be a “MHC binding peptide” or “antigen peptide”. The term “major histocompatibility complex” and the abbreviation “MHC” include MHC class I and MHC class II molecules and relate to a complex of genes which is present in all vertebrates. MHC proteins or molecules are important for signaling between lymphocytes and antigen presenting cells or diseased cells in immune reactions, wherein the MHC proteins or molecules bind peptides and present them for recognition by T cell receptors. The proteins encoded by the MHC are expressed on the surface of cells, and display both self-antigens (peptide fragments from the cell itself) and non-self-antigens (e.g., fragments of invading microorganisms) to a T cell. Preferred such immunogenic portions bind to an MHC class I or class II molecule. As used herein, an immunogenic portion is said to “bind to” an MHC class I or class II molecule if such binding is detectable using any assay known in the art. The term “MHC binding peptide” relates to a peptide which binds to an MHC class I and/or an MHC class II molecule. In the case of class I MHC/peptide complexes, the binding peptides are typically 8-10 amino acids long although longer or shorter peptides may be effective. In the case of class II MHC/peptide complexes, the binding peptides are typically 10-25 amino acids long and are in particular 13-18 amino acids long, whereas longer and shorter peptides may be effective.

In an embodiment, the protein of interest according to the present invention comprises an epitope suitable for vaccination of a target organism. A person skilled in the art will know that one of the principles of immunobiology and vaccination is based on the fact that an immunoprotective reaction to a disease is produced by immunizing an organism with an antigen, which is immunologically relevant with respect to the disease to be treated. According to the present invention, an antigen is selected from the group comprising a self-antigen and non-self-antigen. A non-self-antigen is preferably a bacterial antigen, a virus antigen, a fungus antigen, an allergen or a parasite antigen. It is preferred that the antigen comprises an epitope that is capable of eliciting an immune response in a target organism. For example, the epitope may elicit an immune response against a bacterium, a virus, a fungus, a parasite, an allergen, or a tumor.

In some embodiments the non-self-antigen is a bacterial antigen. In some embodiments, the antigen elicits an immune response against a bacterium which infects animals, including birds, fish and mammals, including domesticated animals. Preferably, the bacterium against which the immune response is elicited is a pathogenic bacterium.

In some embodiments the non-self-antigen is a virus antigen. A virus antigen may for example be a peptide from a virus surface protein, e.g. a capsid polypeptide or a spike polypeptide, such as from Coronavirus. In some embodiments, the antigen elicits an immune response against a virus which infects animals, including birds, fish and mammals, including domesticated animals. Preferably, the virus against which the immune response is elicited is a pathogenic virus.

In some embodiments the non-self-antigen is a polypeptide or a protein from a fungus. In some embodiments, the antigen elicits an immune response against a fungus which infects animals, including birds, fish and mammals, including domesticated animals. Preferably, the fungus against which the immune response is elicited is a pathogenic fungus.

In some embodiments the non-self-antigen is a polypeptide or protein from a unicellular eukaryotic parasite. In some embodiments, the antigen elicits an immune response against a unicellular eukaryotic parasite, preferably a pathogenic unicellular eukaryotic parasite. Pathogenic unicellular eukaryotic parasites may be e.g. from the genus Plasmodium, e.g. P. falciparum, P. vivax, P. malariae or P. ovale, from the genus Leishmania, or from the genus Trypanosoma, e.g. T. cruzi or T. brucei.

In some embodiments the non-self-antigen is an allergenic polypeptide or an allergenic protein. An allergenic protein or allergenic polypeptide is suitable for allergen immunotherapy, also known as hypo-sensitization.

In some embodiments the antigen is a self-antigen, particularly a tumor antigen. Tumor antigens and their determination are known to the skilled person.

In the context of the present invention, the term “tumor antigen” or “tumor-associated antigen” relates to proteins that are under normal conditions specifically expressed in a limited number of tissues and/or organs or in specific developmental stages, for example, the tumor antigen may be under normal conditions specifically expressed in stomach tissue, preferably in the gastric mucosa, in reproductive organs, e.g., in testis, in trophoblastic tissue, e.g., in placenta, or in germ line cells, and are expressed or aberrantly expressed in one or more tumor or cancer tissues. In this context, “a limited number” preferably means not more than 3, more preferably not more than 2. The tumor antigens in the context of the present invention include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. In the context of the present invention, the tumor antigen is preferably associated with the cell surface of a cancer cell and is preferably not or only rarely expressed in normal tissues. Preferably, the tumor antigen or the aberrant expression of the tumor antigen identifies cancer cells. In the context of the present invention, the tumor antigen that is expressed by a cancer cell in a subject, e.g., a patient suffering from a cancer disease, is preferably a self-protein in said subject. In preferred embodiments, the tumor antigen in the context of the present invention is expressed under normal conditions specifically in a tissue or organ that is non-essential, i.e., tissues or organs which when damaged by the immune system do not lead to death of the subject, or in organs or structures of the body which are not or only hardly accessible by the immune system. Preferably, the amino acid sequence of the tumor antigen is identical between the tumor antigen which is expressed in normal tissues and the tumor antigen which is expressed in cancer tissues.

Examples for tumor antigens that may be useful in the present invention are p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, -2, -3, NA88-A, NF1, NY-ESO-1, NY-BR-1, p190 minor BCR-abL, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT. Particularly preferred tumor antigens include CLAUDIN-18.2 (CLDN18.2) and CLAUDIN-6 (CLDN6).

In some embodiments, it is not required that the pharmaceutically active peptide or protein is an antigen that elicits an immune response. Suitable pharmaceutically active proteins or peptides may be selected from the group consisting of cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, chimeric antigen receptors (CARs), immunoglobulins), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthetic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Willebrand factor, antithrombin III, glucocerebrosidase, erythropoietin granulocyte colony stimulating factor (GCSF) or modified Factor VIII, anticoagulants and the like. In one embodiment, the pharmaceutically active protein according to the invention is a cytokine which is involved in regulating lymphoid homeostasis, preferably a cytokine which is involved in and preferably induces or enhances development, priming, expansion, differentiation and/or survival of T cells. In one embodiment, the cytokine is an interleukin, e.g. IL-2, IL-7, IL-12, IL-15, or IL-21.

### Inhibitor of Interferon (IFN) Signaling

A further suitable protein of interest encoded by an open reading frame is an inhibitor of interferon (IFN) signaling. While it has been reported that viability of cells in which RNA has been introduced for expression can be reduced, in particular, if cells are transfected multiple times with RNA, IFN inhibiting agents were found to enhance the viability of cells in which RNA is to be expressed (WO 2014/071963 A1). Preferably, the inhibitor is an inhibitor of IFN type I signaling. Preventing engagement of IFN receptor by extracellular IFN and inhibiting intracellular IFN signaling in the cells allows stable expression of RNA in the cells. Alternatively; or additionally, preventing engagement of IFN receptor by extracellular IFN and inhibiting intracellular IFN signaling enhances survival of the cells, in particular, if cells are transfected repetitively with RNA. Without wishing to be bound by theory, it is envisaged that intracellular IFN signaling can result in inhibition of translation and/or RNA degradation. This can be addressed by inhibiting one or more IFN-inducible antivirally active effector proteins. The IFN-inducible antivirally active effector protein can be selected from the group consisting of RNA-dependent protein kinase (PKR), 2′,5′-oligoadenylate synthetase (OAS) and RNaseL. Inhibiting intracellular IFN signaling may comprise inhibiting the PKR-dependent pathway and/or the OAS-dependent pathway. A suitable protein of interest is a protein that is capable of inhibiting the PKR-dependent pathway and/or the OAS-dependent pathway. Inhibiting the PKR-dependent pathway may comprise inhibiting eIF2-alpha phosphorylation. Inhibiting PKR may comprise treating the cell with at least one PKR inhibitor. The PKR inhibitor may be a viral inhibitor of PKR. The preferred viral inhibitor of PKR is vaccinia virus E3. If a peptide or protein (e.g. E3, K3) is to inhibit intracellular IFN signaling, intracellular expression of the peptide or protein is preferred. Vaccinia virus E3 is a 25 kDa dsRNA-binding protein (encoded by gene E3L) that binds and sequesters dsRNA to prevent the activation of PKR and OAS. E3 can bind directly to PKR and inhibits its activity, resulting in reduced phosphorylation of eIF2-alpha. Other suitable inhibitors of IFN signaling are Herpes simplex virus ICP34.5, Influenza virus NS1, Toscana virus NSs, Bombyx mori nucleopolyhedrovirus PK2, and HCV NS34A.

Position of the at Least One Open Reading Frame in the rRNA Molecule

The rRNA replicon is suitable for expression of one or more genes encoding a peptide of interest or a protein of interest, optionally under control of a subgenomic promoter. Various embodiments are possible. One or more open reading frames, each encoding a peptide of interest or a protein of interest, can be present on the RNA replicon. The most upstream open reading frame of the RNA replicon is referred to as “first open reading frame”. In one embodiment, the first open reading frame encoding a protein of interest is located downstream from the 5′ replication recognition sequence and upstream from the IRES (and the open reading frame encoding a functional non-structural protein from a self-replicating virus). In some embodiments, the “first open reading frame” is the only open reading frame of the RNA replicon. Optionally, one or more further open reading frames can be present downstream of the first open reading frame. One or more further open reading frames downstream of the first open reading frame may be referred to as “second open reading frame”, “third open reading frame” and so on, in the order (5′ to 3′) in which they are present downstream of the first open reading frame. In one embodiment, one or more further open reading frames encoding one or more proteins of interest are located downstream from the open reading frame encoding a functional non-structural protein from a self-replicating virus and are preferably controlled by subgenomic promotors. Preferably, each open reading frame comprises a start codon (base triplet), typically AUG (in the RNA molecule), corresponding to ATG (in a respective DNA molecule).

If the replicon comprises a 3′ replication recognition sequence, it is preferred that all open reading frames are localized upstream of the 3′ replication recognition sequence.

In some embodiments, at least one open reading frame of the replicon is under the control of a subgenomic promoter, preferably an alphavirus subgenomic promoter. The alphavirus subgenomic promoter is very efficient, and is therefore suitable for heterologous gene expression at high levels. Preferably, the subgenomic promoter is a promoter for a subgenomic transcript in an alphavirus. This means that the subgenomic promoter is one which is native to an alphavirus and which preferably controls transcription of the open reading frame encoding one or more structural proteins in said alphavirus. Alternatively, the subgenomic promoter is a variant of a subgenomic promoter of an alphavirus; any variant which functions as promoter for subgenomic RNA transcription in a host cell is suitable. If the replicon comprises a subgenomic promoter, it is preferred that the replicon comprises a conserved sequence element 3 (CSE 3) or a variant thereof.

Preferably, the at least one open reading frame under control of a subgenomic promoter is localized downstream of the subgenomic promoter. Preferably, the subgenomic promoter controls production of subgenomic RNA comprising a transcript of the open reading frame.

In some embodiments the first open reading frame is under control of a subgenomic promoter. In one embodiment, when the first open reading frame is under control of the subgenomic promoter, the gene encoded by the first open reading frame can be expressed both from the replicon as well as from a subgenomic transcript thereof (the latter in the presence of functional alphavirus non-structural protein). One or more further open reading frames, each under control of a subgenomic promoter, may be present downstream of the first open reading frame that may be under control of a subgenomic promoter. The genes encoded by the one or more further open reading frames, e.g. by the second open reading frame, may be translated from one or more subgenomic transcripts, each under control of a subgenomic promoter. For example, the RNA replicon may comprise a subgenomic promoter controlling production of a transcript that encodes a second protein of interest.

In other embodiments the first open reading frame is not under control of a subgenomic promoter. In one embodiment, when the first open reading frame is not under control of a subgenomic promoter, the gene encoded by the first open reading frame can be expressed from the replicon. One or more further open reading frames, each under control of a subgenomic promoter, may be present downstream of the first open reading frame. The genes encoded by the one or more further open reading frames may be expressed from subgenomic transcripts.

In a cell which comprises the replicon according to the present invention, the replicon may be amplified by functional non-structural protein. Additionally, if the replicon comprises one or more open reading frames under control of a subgenomic promoter, one or more subgenomic transcripts are expected to be prepared by functional non-structural protein.

If a replicon comprises more than one open reading frame encoding a protein of interest, it is preferable that each open reading frame encodes a different protein. For example, the protein encoded by the second open reading frame is different from the protein encoded by the first open reading frame.

### Other Features of Replicable RNA Molecules According to the Invention

RNA molecules according to the invention may optionally be characterized by further features, e.g. by a 5′-cap, a 5′-UTR, a 3′-UTR, a poly(A) sequence, and/or adaptation of the codon usage for optimized translation and/or stabilization of the RNA molecule, as detailed below.

### Cap

In some embodiments, the replicon according to the present invention comprises a 5′-cap.

The terms “5′-cap”, “cap”, “5′-cap structure”, “cap structure” are used synonymously to refer to a dinucleotide that is found on the 5′ end of some eukaryotic primary transcripts such as precursor messenger RNA. A 5′-cap is a structure wherein a (optionally modified) guanosine is bonded to the first nucleotide of an mRNA molecule via a 5′ to 5′ triphosphate linkage (or modified triphosphate linkage in the case of certain cap analogs). The terms can refer to a conventional cap or to a cap analog.

“RNA which comprises a 5′-cap” or “RNA which is provided with a 5′-cap” or “RNA which is modified with a 5′-cap” or “capped RNA” refers to RNA which comprises a 5′-cap. For example, providing an RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In one embodiment, the RNA replicon comprises a 5′-cap. In one embodiment, the RNA replicon does not comprise a 5′-cap.

The term “conventional 5′-cap” refers to a naturally occurring 5′-cap, preferably to the 7-methylguanosine cap. In the 7-methylguanosine cap, the guanosine of the cap is a modified guanosine wherein the modification consists of a methylation at the 7-position.

In the context of the present invention, the term “5′-cap analog” refers to a molecular structure that resembles a conventional 5′-cap, but is modified to possess the ability to stabilize RNA if attached thereto, preferably in vivo and/or in a cell. A cap analog is not a conventional 5′-cap.

For the case of eukaryotic mRNA, the 5′-cap has been generally described to be involved in efficient translation of mRNA: in general, in eukaryotes, translation is initiated only at the 5′ end of a messenger RNA (mRNA) molecule, unless an internal ribosomal entry site (IRES) is present. Eukaryotic cells are capable of providing an RNA with a 5′-cap during transcription in the nucleus: newly synthesized mRNAs are usually modified with a 5′-cap structure, e.g.; when the transcript reaches a length of 20 to 30 nucleotides. First, the 5′ terminal nucleotide pppN (ppp representing triphosphate; N representing any nucleoside) is converted in the cell to 5′ GpppN by a capping enzyme having RNA 5′-triphosphatase and guanylyltransferase activities. The GpppN may subsequently be methylated in the cell by a second enzyme with (guanine-7)-methyltransferase activity to form the mono-methylated m⁷GpppN cap. In one embodiment, the 5′-cap used in the present invention is a natural 5′-cap.

In the present invention, a natural 5′-cap dinucleotide is typically selected from the group consisting of a non-methylated cap dinucleotide (G(5′)ppp(5′)N; also termed GpppN) and a methylated cap dinucleotide ((m⁷G(5′)ppp(5′)N; also termed m⁷GpppN). m⁷GpppN (wherein N is G) is represented by the following formula:

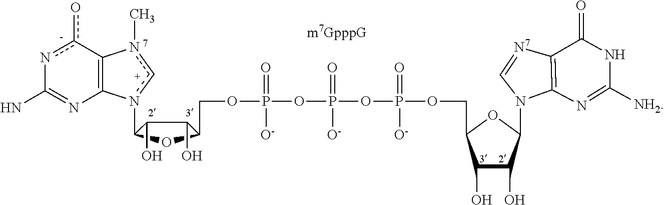

Capped RNA of the present invention can be prepared in vitro, and therefore, does not depend on a capping machinery in a host cell. The most frequently used method to make capped RNAs in vitro is to transcribe a DNA template with either a bacterial or bacteriophage RNA polymerase in the presence of all four ribonucleoside triphosphates and a cap dinucleotide such as m⁷G(5′)ppp(5′)G (also called m⁷GpppG). The RNA polymerase initiates transcription with a nucleophilic attack by the 3′-OH of the guanosine moiety of m⁷GpppG on the α-phosphate of the next templated nucleoside triphosphate (pppN), resulting in the intermediate m⁷GpppGpN (wherein N is the second base of the RNA molecule). The formation of the competing GTP-initiated product pppGpN is suppressed by setting the molar ratio of cap to GTP between 5 and 10 during in vitro transcription.

In preferred embodiments of the present invention, the 5′-cap (if present) is a 5′-cap analog. These embodiments are particularly suitable if the RNA is obtained by in vitro transcription, e.g. is an in vitro transcribed RNA (IVT-RNA). Cap analogs have been initially described to facilitate large scale synthesis of RNA transcripts by means of in vitro transcription.

For messenger RNA, some cap analogs (synthetic caps) have been generally described to date, and they can all be used in the context of the present invention. Ideally, a cap analog is selected that is associated with higher translation efficiency and/or increased resistance to in vivo degradation and/or increased resistance to in vitro degradation.

Preferably, a cap analog is used that can only be incorporated into an RNA chain in one orientation. Pasquinelli et al., 1995, RNA J. 1:957-967) demonstrated that during in vitro transcription, bacteriophage RNA polymerases use the 7-methylguanosine unit for initiation of transcription, whereby around 40-50% of the transcripts with cap possess the cap dinucleotide in a reverse orientation (i.e., the initial reaction product is Gpppm⁷GpN). Compared to the RNAs with a correct cap, RNAs with a reverse cap are not functional with respect to translation of a nucleic acid sequence into protein. Thus, it is desirable to incorporate the cap in the correct orientation, i.e., resulting in an RNA with a structure essentially corresponding to m⁷GpppGpN etc. It has been shown that the reverse integration of the cap-dinucleotide is inhibited by the substitution of either the 2′- or the 3′-OH group of the methylated guanosine unit (Stepinski et al., 2001, RNA J. 7:1486-1495; Peng et al., 2002, Org. Lett. 24:161-164). RNAs which are synthesized in presence of such “anti reverse cap analogs” are translated more efficiently than RNAs which are in vitro transcribed in presence of the conventional 5′-cap m⁷GpppG. To that end, one cap analog in which the 3′ OH group of the methylated guanosine unit is replaced by OCH₃ is described, e.g., by Holtkamp et al., 2006, Blood 108:4009-4017 (7-methyl(3′-O-methyl)GpppG; anti-reverse cap analog (ARCA)). ARCA is a suitable cap dinucleotide according to the present invention.

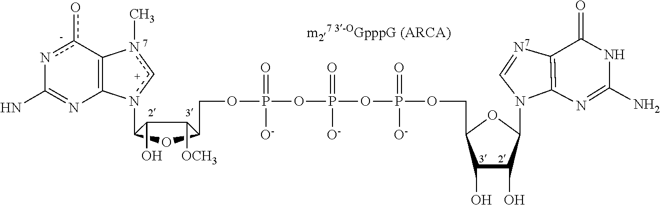

In an embodiment, the RNA of the present invention is essentially not susceptible to decapping. This is important because, in general, the amount of protein produced from synthetic mRNAs introduced into cultured mammalian cells is limited by the natural degradation of mRNA. One in vivo pathway for mRNA degradation begins with the removal of the mRNA cap. This removal is catalyzed by a heterodimeric pyrophosphatase, which contains a regulatory subunit (Dcp1) and a catalytic subunit (Dcp2). The catalytic subunit cleaves between the α and β phosphate groups of the triphosphate bridge. In the present invention, a cap analog may be selected or present that is not susceptible, or less susceptible, to that type of cleavage. A suitable cap analog for this purpose may be selected from a cap dinucleotide according to formula (I):

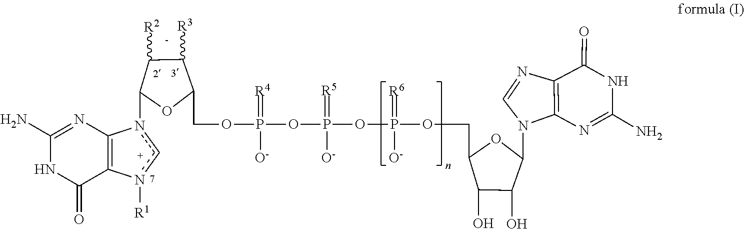

wherein R¹ is selected from the group consisting of optionally substituted alkyl, optionally substituted alkenyl, optionally substituted alkynyl, optionally substituted cycloalkyl, optionally substituted heterocyclyl, optionally substituted aryl, and optionally substituted heteroaryl,
R² and R³ are independently selected from the group consisting of H, halo, OH, and optionally substituted alkoxy, or R² and R³ together form O—X—O, wherein X is selected from the group consisting of optionally substituted CH₂, CH₂CH₂, CH₂CH₂CH₂, CH₂CH(CH₃), and
C(CH₃)₂, or R² is combined with the hydrogen atom at position 4′ of the ring to which R² is attached to form —O—CH₂— or —CH₂—O—,
R⁵ is selected from the group consisting of S, Se, and BH₃,
R⁴ and R⁶ are independently selected from the group consisting of O, S, Se, and BH₃.
n is 1, 2, or 3.

Preferred embodiments for R¹, R², R³, R⁴, R⁵, R⁶ are disclosed in WO 2011/015347 A1 and may be selected accordingly in the present invention.

For example, in an embodiment, the RNA of the present invention comprises a phosphorothioate-cap-analog. Phosphorothioate-cap-analogs are specific cap analogs in which one of the three non-bridging O atoms in the triphosphate chain is replaced with an S atom, i.e., one of R⁴, R⁵ or R⁶ in Formula (I) is S. Phosphorothioate-cap-analogs have been described by Kowalska et al., 2008, RNA, 14:1119-1131, as a solution to the undesired decapping process, and thus to increase the stability of RNA in vivo. In particular, the substitution of an oxygen atom for a sulphur atom at the beta-phosphate group of the 5′-cap results in stabilization against Dcp2. In that embodiment, which is preferred in the present invention, R⁵ in Formula (I) is S; and R⁴ and R⁶ are O.

In a further embodiment, the RNA of the present invention comprises a phosphorothioate-cap-analog wherein the phosphorothioate modification of the RNA 5′-cap is combined with an “anti-reverse cap analog” (ARCA) modification. Respective ARCA-phosphorothioate-cap-analogs are described in WO 2008/157688 A2, and they can all be used in the RNA of the present invention. In that embodiment, at least one of R² or R³ in Formula (I) is not OH, preferably one among R² and R³ is methoxy (OCH₃), and the other one among R² and R³ is preferably OH. In a preferred embodiment, an oxygen atom is substituted for a sulphur atom at the beta-phosphate group (so that R⁵ in Formula (I) is S; and R⁴ and R⁶ are O). It is believed that the phosphorothioate modification of the ARCA ensures that the α, β, and γ phosphorothioate groups are precisely positioned within the active sites of cap-binding proteins in both the translational and decapping machinery. At least some of these analogs are essentially resistant to pyrophosphatase Dcp1/Dcp2. Phosphorothioate-modified ARCAs were described to have a much higher affinity for eIF4E than the corresponding ARCAs lacking a phosphorothioate group.

A respective cap analog that is particularly preferred in the present invention, i.e., m_{2′}^{7,2′-O}GppₛpG, is termed beta-S-ARCA (WO 2008/157688 A2; Kuhn et al., 2010, Gene Ther. 17:961-971). Thus, in one embodiment of the present invention, the RNA of the present invention is modified with beta-S-ARCA. beta-S-ARCA is represented by the following structure:

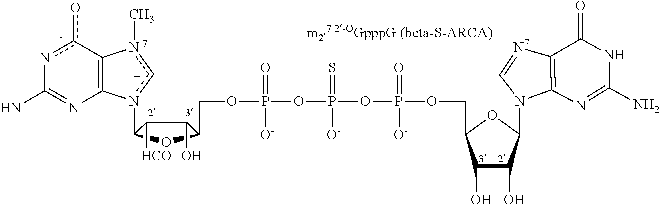

In general, the replacement of an oxygen atom for a sulphur atom at a bridging phosphate results in phosphorothioate diastereomers which are designated D1 and D2, based on their elution pattern in HPLC. Briefly, the D1 diastereomer of beta-S-ARCA” or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time. Determination of the stereochemical configuration by HPLC is described in WO 2011/015347 A1.

In a first particularly preferred embodiment of the present invention, RNA of the present invention is modified with the beta-S-ARCA(D2) diastereomer. The two diastereomers of beta-S-ARCA differ in sensitivity against nucleases. It has been shown that RNA carrying the D2 diastereomer of beta-S-ARCA is almost fully resistant against Dcp2 cleavage (only 6% cleavage compared to RNA which has been synthesized in presence of the unmodified ARCA 5′-cap), whereas RNA with the beta-S-ARCA(D1) 5′-cap exhibits an intermediary sensitivity to Dcp2 cleavage (71% cleavage). It has further been shown that the increased stability against Dcp2 cleavage correlates with increased protein expression in mammalian cells. In particular, it has been shown that RNAs carrying the beta-S-ARCA(D2) cap are more efficiently translated in mammalian cells than RNAs carrying the beta-S-ARCA(D1) cap. Therefore, in one embodiment of the present invention, RNA of the present invention is modified with a cap analog according to Formula (I), characterized by a stereochemical configuration at the P atom comprising the substituent R⁵ in Formula (I) that corresponds to that at the P_{β} atom of the D2 diastereomer of beta-S-ARCA. In that embodiment, R⁵ in Formula (I) is S; and R⁴ and R⁶ are O. Additionally, at least one of R² or R³ in Formula (I) is preferably not OH, preferably one among R² and R³ is methoxy (OCH3), and the other one among R² and R³ is preferably OH.

In a second particularly preferred embodiment, RNA of the present invention is modified with the beta-S-ARCA(D1) diastereomer. This embodiment is particularly suitable for transfer of capped RNA into immature antigen presenting cells, such as for vaccination purposes. It has been demonstrated that the beta-S-ARCA(D1) diastereomer, upon transfer of respectively capped RNA into immature antigen presenting cells, is particularly suitable for increasing the stability of the RNA, increasing translation efficiency of the RNA, prolonging translation of the RNA, increasing total protein expression of the RNA, and/or increasing the immune response against an antigen or antigen peptide encoded by said RNA (Kuhn et al., 2010, Gene Ther. 17:961-971). Therefore, in an alternative embodiment of the present invention, RNA of the present invention is modified with a cap analog according to Formula (I), characterized by a stereochemical configuration at the P atom comprising the substituent R⁵ in Formula (I) that corresponds to that at the P_{β} atom of the D1 diastereomer of beta-S-ARCA. Respective cap analogs and embodiments thereof are described in WO 2011/015347 A1 and Kuhn et al., 2010, Gene Ther. 17:961-971. Any cap analog described in WO 2011/015347 A1, wherein the stereochemical configuration at the P atom comprising the substituent R⁵ corresponds to that at the P_{β} atom of the D1 diastereomer of beta-S-ARCA, may be used in the present invention. Preferably, R⁵ in Formula (I) is S; and R⁴ and R⁶ are O. Additionally, at least one of R² or R³ in Formula (I) is preferably not OH, preferably one among R² and R³ is methoxy (OCH3), and the other one among R² and R³ is preferably OH.

In one embodiment, RNA of the present invention is modified with a 5′-cap structure according to Formula (I) wherein any one phosphate group is replaced by a boranophosphate group or a phosphoroselenoate group. Such caps have increased stability both in vitro and in vivo. Optionally, the respective compound has a 2′-O- or 3′-O-alkyl group (wherein alkyl is preferably methyl); respective cap analogs are termed BH₃-ARCAs or Se-ARCAs. Compounds that are particularly suitable for capping of mRNA include the 3-BH₃-ARCAs and R-Se-ARCAs, as described in WO 2009/149253 A2. For these compounds, a stereochemical configuration at the P atom comprising the substituent R⁵ in Formula (I) that corresponds to that at the P_{β} atom of the D1 diastereomer of beta-S-ARCA is preferred.

In one embodiment, the 5′ cap can have the following structure:

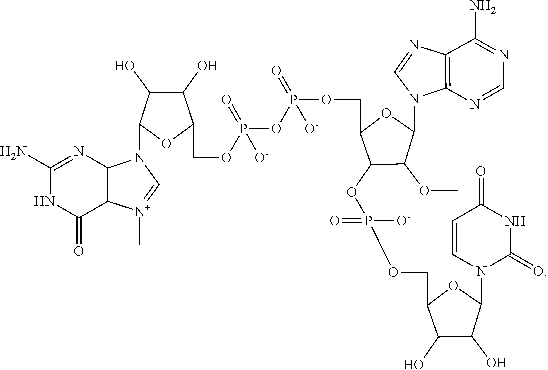 [SMILES: COC1C(OP(=O)([O-])OCC2OC(n3ccc(=O)[nH]c3=O)C(O)C2O)C(COP(=O)([O-])OP(=O)([O-])OCC2OC(N3C=[N+](C)C4C(=O)NC(N)=NC43)C(O)C2O)OC1n1cnc2c(N)ncnc21]

In embodiments in which this type of cap is used, the U corresponding to the second nucleotide of alphaviruses is excluded from modification, for example, excluded from modification to N1-methyl-pseudouridine.

### UTR

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR).

A 3′-UTR, if present, is located at the 3′ end of a gene, downstream of the termination codon of a protein-encoding region, but the term “3′-UTR” does preferably not include the poly(A) tail. Thus, the 3′-UTR is upstream of the poly(A) tail (if present), e.g. directly adjacent to the poly(A) tail.

A 5′-UTR, if present, is located at the 5′ end of a gene, upstream of the start codon of a protein-encoding region. A 5′-UTR is downstream of the 5′-cap (if present), e.g. directly adjacent to the 5′-cap.

5′- and/or 3′-untranslated regions may, according to the invention, be functionally linked to an open reading frame, so as for these regions to be associated with the open reading frame in such a way that the stability and/or translation efficiency of the RNA comprising said open reading frame are increased.

In some embodiments, the RNA replicon according to the present invention comprises a 5′-UTR and/or a 3′-UTR.

UTRs are implicated in stability and translation efficiency of RNA. Both can be improved, besides structural modifications concerning the 5′-cap and/or the 3′ poly(A)-tail as described herein, by selecting specific 5′ and/or 3′ untranslated regions (UTRs). Sequence elements within the UTRs are generally understood to influence translational efficiency (mainly 5′-UTR) and RNA stability (mainly 3′-UTR). It is preferable that a 5′-UTR is present that is active in order to increase the translation efficiency and/or stability of the RNA replicon. Independently or additionally, it is preferable that a 3′-UTR is present that is active in order to increase the translation efficiency and/or stability of the RNA replicon.

The terms “active in order to increase the translation efficiency” and/or “active in order to increase the stability”, with reference to a first nucleic acid sequence (e.g. a UTR), means that the first nucleic acid sequence is capable of modifying, in a common transcript with a second nucleic acid sequence, the translation efficiency and/or stability of said second nucleic acid sequence in such a way that said translation efficiency and/or stability is increased in comparison with the translation efficiency and/or stability of said second nucleic acid sequence in the absence of said first nucleic acid sequence.

In one embodiment, the RNA replicon according to the present invention comprises a 5′-UTR and/or a 3′-UTR which is heterologous or non-native to the alphavirus from which the functional alphavirus non-structural protein is derived. This allows the untranslated regions to be designed according to the desired translation efficiency and RNA stability. Thus, heterologous or non-native UTRs allow for a high degree of flexibility, and this flexibility is advantageous compared to native alphaviral UTRs.

Preferably, the RNA replicon according to the present invention comprises a 5′-UTR and/or a 3′-UTR that is not of virus origin; particularly not of alphavirus origin. In one embodiment, the RNA replicon comprises a 5′-UTR derived from a eukaryotic 5′-UTR and/or a 3′-UTR derived from a eukaryotic 3′-UTR.

A 5′-UTR according to the present invention can comprise any combination of more than one nucleic acid sequence, optionally separated by a linker. A 3′-UTR according to the present invention can comprise any combination of more than one nucleic acid sequence, optionally separated by a linker.

The term “linker” according to the invention relates to a nucleic acid sequence added between two nucleic acid sequences to connect said two nucleic acid sequences. There is no particular limitation regarding the linker sequence.

A 3′-UTR typically has a length of 200 to 2000 nucleotides, e.g. 500 to 1500 nucleotides. The 3′-untranslated regions of immunoglobulin mRNAs are relatively short (fewer than about 300 nucleotides), while the 3′-untranslated regions of other genes are relatively long. For example, the 3′-untranslated region of tPA is about 800 nucleotides in length, that of factor VIII is about 1800 nucleotides in length and that of erythropoietin is about 560 nucleotides in length. The Y-untranslated regions of mammalian mRNA typically have a homology region known as the AAUAAA hexanucleotide sequence. This sequence is presumably the poly(A) attachment signal and is frequently located from 10 to 30 bases upstream of the poly(A) attachment site. Y-untranslated regions may contain one or more inverted repeats which can fold to give stem-loop structures which act as barriers for exoribonucleases or interact with proteins known to increase RNA stability (e.g. RNA-binding proteins).

The human beta-globin 3′-UTR, particularly two consecutive identical copies of the human beta-globin 3′-UTR, contributes to high transcript stability and translational efficiency (Holtkamp et al., 2006, Blood 108:4009-4017). Thus, in one embodiment, the RNA replicon according to the present invention comprises two consecutive identical copies of the human beta-globin 3′-UTR. Thus, it comprises in the 5′→3′ direction: (a) optionally a 5′-UTR; (b) an open reading frame; (c) a 3′-UTR; said 3′-UTR comprising two consecutive identical copies of the human beta-globin 3′-UTR, a fragment thereof, or a variant of the human beta-globin 3′-UTR or fragment thereof.

In an embodiment, the RNA replicon according to the present invention comprises a 3′-UTR which is active in order to increase translation efficiency and/or stability, but which is not the human beta-globin 3′-UTR, a fragment thereof, or a variant of the human beta-globin 3′-UTR or fragment thereof.

In an embodiment, the RNA replicon according to the present invention comprises a 5′-UTR which is active in order to increase translation efficiency and/or stability.

### Poly(A) Sequence

In some embodiments, the replicon according to the present invention comprises a 3′-poly(A) sequence. If the replicon comprises conserved sequence element 4 (CSE 4), the 3′-poly(A) sequence of the replicon is preferably present downstream of CSE 4, most preferably directly adjacent to CSE 4.

According to the invention, in one embodiment, a poly(A) sequence comprises or essentially consists of or consists of at least 20, preferably at least 26, preferably at least 40, preferably at least 80, preferably at least 100 and preferably up to 500, preferably up to 400, preferably up to 300, preferably up to 200, and in particular up to 150, A nucleotides, and in particular about 120 A nucleotides. In this context “essentially consists of” means that most nucleotides in the poly(A) sequence, typically at least 50%, and preferably at least 75% by number of nucleotides in the “poly(A) sequence”, are A nucleotides (adenylate), but permits that remaining nucleotides are nucleotides other than A nucleotides, such as U nucleotides (uridylate), G nucleotides (guanylate), C nucleotides (cytidylate). In this context “consists of” means that all nucleotides in the poly(A) sequence, i.e. 100% by number of nucleotides in the poly(A) sequence, are A nucleotides. The term “A nucleotide” or “A” refers to adenylate.

Indeed, it has been demonstrated that a 3′ poly(A) sequence of about 120 A nucleotides has a beneficial influence on the levels of RNA in transfected eukaryotic cells, as well as on the levels of protein that is translated from an open reading frame that is present upstream (5′) of the 3′ poly(A) sequence (Holtkamp et al., 2006, Blood, vol. 108, pp. 4009-4017).

In alphaviruses, a 3′ poly(A) sequence of at least 11 consecutive adenylate residues, or at least 25 consecutive adenylate residues, is thought to be important for efficient synthesis of the minus strand. In particular, in alphaviruses, a 3′ poly(A) sequence of at least 25 consecutive adenylate residues is understood to function together with conserved sequence element 4 (CSE 4) to promote synthesis of the (−) strand (Hardy & Rice, 2005, J. Virol. 79:4630-4639).

The present invention provides for a 3′ poly(A) sequence to be attached during RNA transcription, i.e. during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In a preferred embodiment of the present invention, the 3′ poly(A) cassette present in the coding strand of DNA essentially consists of dA nucleotides, but is interrupted by a random sequence having an equal distribution of the four nucleotides (dA, dC, dG, dT). Such random sequence may be 5 to 50, preferably 10 to 30, more preferably 10 to 20 nucleotides in length. Such a cassette is disclosed in WO 2016/005004 A1. Any poly(A) cassette disclosed in WO 2016/005004 A1 may be used in the present invention. A poly(A) cassette that essentially consists of dA nucleotides, but is interrupted by a random sequence having an equal distribution of the four nucleotides (dA, dC, dG, dT) and having a length of, e.g., 5 to 50 nucleotides shows, on DNA level, constant propagation of plasmid DNA in E. coli and is still associated, on RNA level, with the beneficial properties with respect to supporting RNA stability and translational efficiency.

Consequently, in a preferred embodiment of the present invention, the 3′ poly(A) sequence contained in an RNA molecule described herein essentially consists of A nucleotides, but is interrupted by a random sequence having an equal distribution of the four nucleotides (A, C, G, U). Such random sequence may be 5 to 50, preferably 10 to 30, more preferably 10 to 20 nucleotides in length.

### Codon Usage

In general, the degeneracy of the genetic code will allow the substitution of certain codons (base triplets coding for an amino acid) that are present in an RNA sequence by other codons (base triplets), while maintaining the same coding capacity (so that the replacing codon encodes the same amino acid as the replaced codon). In some embodiments of the present invention, at least one codon of an open reading frame comprised by an RNA (rRNA) molecule differs from the respective codon in the respective open reading frame in the species from which the open reading frame originates. In that embodiment, the coding sequence of the open reading frame is said to be “adapted” or “modified”. The coding sequence of an open reading frame comprised by the replicon may be adapted.

For example, when the coding sequence of an open reading frame is adapted, frequently used codons may be selected: WO 2009/024567 A1 describes the adaptation of a coding sequence of a nucleic acid molecule, involving the substitution of rare codons by more frequently used codons. Since the frequency of codon usage depends on the host cell or host organism, that type of adaptation is suitable to fit a nucleic acid sequence to expression in a particular host cell or host organism. Generally, speaking, more frequently used codons are typically translated more efficiently in a host cell or host organism, although adaptation of all codons of an open reading frame is not always required.

For example, when the coding sequence of an open reading frame is adapted, the content of G (guanylate) residues and C (cytidylate) residues may be altered by selecting codons with the highest GC-rich content for each amino acid. RNA molecules with GC-rich open reading frames were reported to have the potential to reduce immune activation and to improve translation and half-life of RNA (Thess et al., 2015, Mol. Ther. 23:1457-1465).

In particular, the coding sequence for non-structural protein can be adapted as desired. This freedom is possible because the open reading frame encoding non-structural protein does not overlap with the 5′ replication recognition sequence of the replicon.

### Safety Features of Embodiments of the Present Invention

The following features are preferred in the present invention, alone or in any suitable combination:

The replicon of the present invention is not particle-forming. This means that, following inoculation of a host cell by the replicon of the present invention, the host cell does not produce virus particles, such as next generation virus particles. In one embodiment, the RNA replicon according to the invention is completely free of genetic information encoding any virus structural protein, e.g., alphavirus structural protein, such as core nucleocapsid protein C, envelope protein P62, and/or envelope protein E1. Preferably, the replicon according to the present invention does not comprise a virus packaging signal, e.g., an alphavirus packaging signal. For example, the alphavirus packaging signal comprised in the coding region of nsP2 of SFV (White et al., 1998, J. Virol. 72:4320-4326) may be removed, e.g. by deletion or mutation. A suitable way of removing the alphavirus packaging signal includes adaptation of the codon usage of the coding region of nsP2. The degeneration of the genetic code may allow to delete the function of the packaging signal without affecting the amino acid sequence of the encoded nsP2.

### DNA

The present invention also provides a DNA comprising a nucleic acid sequence encoding the RNA replicon according to the present invention.

Preferably, the DNA is double-stranded.

In a preferred embodiment, the DNA is a plasmid. The term “plasmid”, as used herein, generally relates to a construct of extrachromosomal genetic material, usually a circular DNA duplex, which can replicate independently of chromosomal DNA.

The DNA of the present invention may comprise a promoter that can be recognized by a DNA-dependent RNA-polymerase. This allows for transcription of the encoded RNA in vivo or in vitro, e.g. of the RNA of the present invention. IVT vectors may be used in a standardized manner as template for in vitro transcription. Examples of promoters preferred according to the invention are promoters for SP6, T3 or T7 polymerase.

In one embodiment, the DNA of the present invention is an isolated nucleic acid molecule.

### Methods of Preparing RNA

The RNA molecule according to the present invention may be obtainable by in vitro transcription. In vitro-transcribed RNA (IVT-RNA) is of particular interest in the present invention. IVT-RNA is obtainable by transcription from a nucleic acid molecule (particularly a DNA molecule). The DNA molecule(s) of the present invention are suitable for such purposes, particularly if comprising a promoter that can be recognized by a DNA-dependent RNA-polymerase.

rRNA according to the present invention can be synthesized in vitro. This allows to add cap-analogs to the in vitro transcription reaction. Typically, the poly(A) tail is encoded by a poly-(dT) sequence on the DNA template. Alternatively, capping and poly(A) tail addition can be achieved enzymatically after transcription.

The in vitro transcription methodology is known to the skilled person. For example, as mentioned in WO 2011/015347 A1, a variety of in vitro transcription kits is commercially available.

### Kit

The present invention also provides a kit comprising an RNA replicon according to the invention.

In one embodiment, the constituents of the kit are present as separate entities. For example, one constituent of the kit may be present in one entity, and another constituent of the kit may be present in a separate entity. For example, an open or closed container is a suitable entity. A closed container is preferred. The container used should preferably be RNAse-free or essentially RNAse-free.

In one embodiment, the kit of the present invention comprises RNA for inoculation with a cell and/or for administration to a human or animal subject.

The kit according to the present invention optionally comprises a label or other form of information element, e.g. an electronic data carrier. The label or information element preferably comprises instructions, e.g. printed written instructions or instructions in electronic form that are optionally printable. The instructions may refer to at least one suitable possible use of the kit.

### Pharmaceutical Composition

The RNA replicon described herein may be present in the form of a pharmaceutical composition. A pharmaceutical composition according to the invention may comprise at least one nucleic acid molecule according to the present invention. A pharmaceutical composition according to the invention comprises a pharmaceutically acceptable diluent and/or a pharmaceutically acceptable excipient and/or a pharmaceutically acceptable carrier and/or a pharmaceutically acceptable vehicle. The choice of pharmaceutically acceptable carrier, vehicle, excipient or diluent is not particularly limited. Any suitable pharmaceutically acceptable carrier, vehicle, excipient or diluent known in the art may be used.

In one embodiment of the present invention, a pharmaceutical composition can further comprise a solvent such as an aqueous solvent or any solvent that makes it possible to preserve the integrity of the rRNA. In a preferred embodiment, the pharmaceutical composition is an aqueous solution comprising RNA. The aqueous solution may optionally comprise solutes, e.g. salts.

In one embodiment of the present invention, the pharmaceutical composition is in the form of a freeze-dried composition. A freeze-dried composition is obtainable by freeze-drying a respective aqueous composition.

In one embodiment, the pharmaceutical composition comprises at least one cationic entity. In general, cationic lipids, cationic polymers and other substances with positive charges may form complexes with negatively charged nucleic acids. It is possible to stabilize the RNA according to the invention by complexation with cationic compounds, preferably polycationic compounds such as for example a cationic or polycationic peptide or protein. In one embodiment, the pharmaceutical composition according to the present invention comprises at least one cationic molecule selected from the group consisting protamine, polyethylene imine, a poly-L-lysine, a poly-L-arginine, a histone or a cationic lipid.

According to the present invention, a cationic lipid is a cationic amphiphilic molecule, e.g., a molecule which comprises at least one hydrophilic and lipophilic moiety. The cationic lipid can be monocationic or polycationic. Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Cationic lipids also include lipids with a tertiary amine group, including 1,2-dilinoleyloxy-N,N-dimethyl-3-aminopropane (DLinDMA). Cationic lipids are suitable for formulating RNA in lipid formulations as described herein, such as liposomes, emulsions and lipoplexes. Typically, positive charges are contributed by at least one cationic lipid and negative charges are contributed by the RNA. In one embodiment, the pharmaceutical composition comprises at least one helper lipid, in addition to a cationic lipid. The helper lipid may be a neutral or an anionic lipid. The helper lipid may be a natural lipid, such as a phospholipid, or an analogue of a natural lipid, or a fully synthetic lipid, or lipid-like molecule, with no similarities with natural lipids. In the case where a pharmaceutical composition includes both a cationic lipid and a helper lipid, the molar ratio of the cationic lipid to the neutral lipid can be appropriately determined in view of stability of the formulation and the like.

In one embodiment, the pharmaceutical composition according to the present invention comprises protamine. According to the invention, protamine is useful as cationic carrier agent. The term “protamine” refers to any of various strongly basic proteins of relatively low molecular weight that are rich in arginine and are found associated especially with DNA in place of somatic histones in the sperm cells of animals such as fish. In particular, the term “protamine” refers to proteins found in fish sperm that are strongly basic, are soluble in water, are not coagulated by heat, and comprise multiple arginine monomers. According to the invention, the term “protamine” as used herein is meant to comprise any protamine amino acid sequence obtained or derived from native or biological sources including fragments thereof and multimeric forms of said amino acid sequence or fragment thereof. Furthermore, the term encompasses (synthesized) polypeptides which are artificial and specifically designed for specific purposes and cannot be isolated from native or biological sources.

In some embodiments, the compositions of the invention may comprise one or more adjuvants. Adjuvants may be added to vaccines to stimulate the immune system's response; adjuvants do not typically provide immunity themselves. Exemplary adjuvants include without limitation the following: Inorganic compounds (e.g. alum, aluminum hydroxide, aluminum phosphate, calcium phosphate hydroxide); mineral oil (e.g. paraffin oil), cytokines (e.g. IL-1, IL-2, IL-12); immunostimulatory polynucleotide (such as RNA or DNA; e.g., CpG-containing oligonucleotides); saponins (e.g. plant saponins from Quillaja, Soybean, Polygala senega); oil emulsions or liposomes; polyoxy ethylene ether and poly oxy ethylene ester formulations; polyphosphazene (PCPP); muramyl peptides; imidazoquinolone compounds; thiosemicarbazone compounds; the Flt3 ligand (WO 2010/066418 A1); or any other adjuvant that is known by a person skilled in the art. A preferred adjuvant for administration of RNA according to the present invention is the Flt3 ligand (WO 2010/066418 A1). When Flt3 ligand is administered together with RNA that codes for an antigen, a strong increase in antigen-specific CD8+ T cells may be observed.

The pharmaceutical composition according to the invention can be buffered, (e.g., with an acetate buffer, a citrate buffer, a succinate buffer, a Tris buffer, a phosphate buffer).

### RNA-Containing Particles

In some embodiments, owing to the instability of non-protected RNA, it is advantageous to provide the RNA molecules of the present invention in complexed or encapsulated form. Respective pharmaceutical compositions are provided in the present invention. In particular, in some embodiments, the pharmaceutical composition of the present invention comprises nucleic acid-containing particles, preferably RNA-containing particles. Respective pharmaceutical compositions are referred to as particulate formulations. In particulate formulations according to the present invention, a particle comprises nucleic acid according to the invention and a pharmaceutically acceptable carrier or a pharmaceutically acceptable vehicle that is suitable for delivery of the nucleic acid. The nucleic acid-containing particles may be, for example, in the form of proteinaceous particles or in the form of lipid-containing particles. Suitable proteins or lipids are referred to as particle forming agents. Proteinaceous particles and lipid-containing particles have been described previously to be suitable for delivery of alphaviral RNA in particulate form (e.g. Strauss & Strauss, 1994, Microbiol. Rev. 58:491-562). In particular, alphavirus structural proteins (provided e.g. by a helper virus) are a suitable carrier for delivery of RNA in the form of proteinaceous particles.

In one embodiment, the particulate formulation of the present invention is a nanoparticulate formulation. In that embodiment, the composition according to the present invention comprises nucleic acid according to the invention in the form of nanoparticles. Nanoparticulate formulations can be obtained by various protocols and with various complexing compounds. Lipids, polymers, oligomers, or amphipiles are typical constituents of nanoparticulate formulations.

As used herein, the term “nanoparticle” refers to any particle having a diameter making the particle suitable for systemic, in particular parenteral, administration, of, in particular, nucleic acids, typically a diameter of 1000 nanometers (nm) or less. In one embodiment, the nanoparticles have an average diameter in the range of from about 50 nm to about 1000 nm, preferably from about 50 nm to about 400 nm, preferably about 100 nm to about 300 nm such as about 150 nm to about 200 nm. In one embodiment, the nanoparticles have a diameter in the range of about 200 to about 700 nm, about 200 to about 600 nm, preferably about 250 to about 550 nm, in particular about 300 to about 500 nm or about 200 to about 400 nm.

In one embodiment, the polydispersity index (PI) of the nanoparticles described herein, as measured by dynamic light scattering, is 0.5 or less, preferably 0.4 or less or even more preferably 0.3 or less. The “polydispersity index” (PI) is a measurement of homogeneous or heterogeneous size distribution of the individual particles (such as liposomes) in a particle mixture and indicates the breadth of the particle distribution in a mixture. The PI can be determined, for example, as described in WO 2013/143555 A1.

As used herein, the term “nanoparticulate formulation” or similar terms refer to any particulate formulation that contains at least one nanoparticle. In some embodiments, a nanoparticulate composition is a uniform collection of nanoparticles. In some embodiments, a nanoparticulate composition is a lipid-containing pharmaceutical formulation, such as a liposome formulation or an emulsion.

### Lipid-Containing Pharmaceutical Compositions

In one embodiment, the pharmaceutical composition of the present invention comprises at least one lipid. Preferably, at least one lipid is a cationic lipid. Said lipid-containing pharmaceutical composition comprises nucleic acid according to the present invention. In one embodiment, the pharmaceutical composition according to the invention comprises RNA encapsulated in a vesicle, e.g. in a liposome. In one embodiment, the pharmaceutical composition according to the invention comprises RNA in the form of an emulsion. In one embodiment, the pharmaceutical composition according to the invention comprises rRNA in a complex with a cationic compound, thereby forming e.g. so-called lipoplexes or polyplexes. Encapsulation of RNA within vesicles such as liposomes is distinct from, for instance, lipid/RNA complexes. Lipid/RNA complexes are obtainable e.g. when RNA is e.g. mixed with pre-formed liposomes.

In one embodiment, the pharmaceutical composition according to the invention comprises rRNA encapsulated in a vesicle. Such formulation is a particular particulate formulation according to the invention. A vesicle is a lipid bilayer rolled up into a spherical shell, enclosing a small space and separating that space from the space outside the vesicle. Typically, the space inside the vesicle is an aqueous space, i.e. comprises water. Typically, the space outside the vesicle is an aqueous space, i.e. comprises water. The lipid bilayer is formed by one or more lipids (vesicle-forming lipids). The membrane enclosing the vesicle is a lamellar phase, similar to that of the plasma membrane. The vesicle according to the present invention may be a multilamellar vesicle, a unilamellar vesicle, or a mixture thereof. When encapsulated in a vesicle, the rRNA is typically separated from any external medium. Thus, it is present in protected form, functionally equivalent to the protected form in a natural alphavirus. Suitable vesicles are particles, particularly nanoparticles, as described herein.

For example, RNA (rRNA) may be encapsulated in a liposome. In that embodiment, the pharmaceutical composition is or comprises a liposome formulation. Encapsulation within a liposome will typically protect RNA from RNase digestion. It is possible that the liposomes include some external RNA (e.g. on their surface), but at least half of the RNA (and ideally all of it) is encapsulated within the core of the liposome.

Liposomes are microscopic lipidic vesicles often having one or more bilayers of a vesicle-forming lipid, such as a phospholipid, and are capable of encapsulating a drug, e.g. RNA. Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. Such phases may be present in nanoparticulate RNA formulations of the present invention.

Liposomes may be formed using standard methods known to the skilled person. Respective methods include the reverse evaporation method, the ethanol injection method, the dehydration-rehydration method, sonication or other suitable methods. Following liposome formation, the liposomes can be sized to obtain a population of liposomes having a substantially homogeneous size range.

In a preferred embodiment of the present invention, the rRNA is present in a liposome which includes at least one cationic lipid. Respective liposomes can be formed from a single lipid or from a mixture of lipids, provided that at least one cationic lipid is used. Preferred cationic lipids have a nitrogen atom which is capable of being protonated; preferably, such cationic lipids are lipids with a tertiary amine group. A particularly suitable lipid with a tertiary amine group is 1,2-dilinoleyloxy-N,N-dimethyl-3-aminopropane (DLinDMA). In one embodiment, the RNA according to the present invention is present in a liposome formulation as described in WO 2012/006378 A1: a liposome having a lipid bilayer encapsulating an aqueous core including RNA, wherein the lipid bilayer comprises a lipid with a pKa in the range of 5.0 to 7.6, which preferably has a tertiary amine group. Preferred cationic lipids with a tertiary amine group include DLinDMA (pKa 5.8) and are generally described in WO 2012/031046 A2. According to WO 2012/031046 A2, liposomes comprising a respective compound are particularly suitable for encapsulation of RNA and thus liposomal delivery of RNA. In one embodiment, the RNA according to the present invention is present in a liposome formulation, wherein the liposome includes at least one cationic lipid whose head group includes at least one nitrogen atom (N) which is capable of being protonated, wherein the liposome and the RNA have a N:P ratio of between 1:1 and 20:1. According to the present invention, “N:P ratio” refers to the molar ratio of nitrogen atoms (N) in the cationic lipid to phosphate atoms (P) in the RNA comprised in a lipid containing particle (e.g. liposome), as described in WO 2013/006825 A1. The N:P ratio of between 1:1 and 20:1 is implicated in the net charge of the liposome and in efficiency of delivery of RNA to a vertebrate cell.

In one embodiment, the rRNA according to the present invention is present in a liposome formulation that comprises at least one lipid which includes a polyethylene glycol (PEG) moiety, wherein RNA is encapsulated within a PEGylated liposome such that the PEG moiety is present on the liposome's exterior, as described in WO 2012/031043 A1 and WO 2013/033563 A1.

In one embodiment, the rRNA according to the present invention is present in a liposome formulation, wherein the liposome has a diameter in the range of 60-180 nm, as described in WO 2012/030901 A1.

In one embodiment, the rRNA according to the present invention is present in a liposome formulation, wherein the rRNA-containing liposomes have a net charge close to zero or negative, as disclosed in WO 2013/143555 A1.

In other embodiments, the rRNA according to the present invention is present in the form of an emulsion. Emulsions have been previously described to be used for delivery of nucleic acid molecules, such as rRNA molecules, to cells. Preferred herein are oil-in-water emulsions. The respective emulsion particles comprise an oil core and a cationic lipid. More preferred are cationic oil-in-water emulsions in which the RNA according to the present invention is complexed to the emulsion particles. The emulsion particles comprise an oil core and a cationic lipid. The cationic lipid can interact with the negatively charged rRNA, thereby anchoring the rRNA to the emulsion particles. In an oil-in-water emulsion, emulsion particles are dispersed in an aqueous continuous phase. For example, the average diameter of the emulsion particles may typically be from about 80 nm to 180 nm. In one embodiment, the pharmaceutical composition of the present invention is a cationic oil-in-water emulsion, wherein the emulsion particles comprise an oil core and a cationic lipid, as described in WO 2012/006380 A2. The rRNA according to the present invention may be present in the form of an emulsion comprising a cationic lipid wherein the N:P ratio of the emulsion is at least 4:1, as described in WO 2013/006834 A1. The rRNA according to the present invention may be present in the form of a cationic lipid emulsion, as described in WO 2013/006837 A1. In particular, the composition may comprise rRNA complexed with a particle of a cationic oil-in-water emulsion, wherein the ratio of oil/lipid is at least about 8:1 (mole:mole).

In other embodiments, the pharmaceutical composition according to the invention comprises RNA in the format of a lipoplex. The term, “lipoplex” or “RNA lipoplex” refers to a complex of lipids and nucleic acids such as RNA. Lipoplexes can be formed of cationic (positively charged) liposomes and the anionic (negatively charged) nucleic acid. The cationic liposomes can also include a neutral “helper” lipid. In the simplest case, the lipoplexes form spontaneously by mixing the nucleic acid with the liposomes with a certain mixing protocol, however various other protocols may be applied. It is understood that electrostatic interactions between positively charged liposomes and negatively charged nucleic acid are the driving force for the lipoplex formation (WO 2013/143555 A1). In one embodiment of the present invention, the net charge of the RNA lipoplex particles is close to zero or negative. It is known that electro-neutral or negatively charged lipoplexes of RNA and liposomes lead to substantial RNA expression in spleen dendritic cells (DCs) after systemic administration and are not associated with the elevated toxicity that has been reported for positively charged liposomes and lipoplexes (cf. WO 2013/143555 A1). Therefore, in one embodiment of the present invention, the pharmaceutical composition according to the invention comprises RNA in the format of nanoparticles, preferably lipoplex nanoparticles, in which (i) the number of positive charges in the nanoparticles does not exceed the number of negative charges in the nanoparticles and/or (ii) the nanoparticles have a neutral or net negative charge and/or (iii) the charge ratio of positive charges to negative charges in the nanoparticles is 1.4:1 or less and/or (iv) the zeta potential of the nanoparticles is 0 or less. As described in WO 2013/143555 A1, zeta potential is a scientific term for electrokinetic potential in colloidal systems. In the present invention, (a) the zeta potential and (b) the charge ratio of the cationic lipid to the RNA in the nanoparticles can both be calculated as disclosed in WO 2013/143555 A1. In summary, pharmaceutical compositions which are nanoparticulate lipoplex formulations with a defined particle size, wherein the net charge of the particles is close to zero or negative, as disclosed in WO 2013/143555 A1, are preferred pharmaceutical compositions in the context of the present invention.

In one embodiment, nucleic acid such as the rRNA described herein is administered in the form of lipid nanoparticles (LNPs). The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid, a neutral lipid, a steroid, a polymer conjugated lipid; and the RNA, encapsulated within or associated with the lipid nanoparticle.

In one embodiment, the LNP comprises from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In one embodiment, the LNP comprises about 47.0, 47.1, 47.2, 47.3, 47.4, 47.5, 47.6, 47.7, 47.8, 47.9 or 48.0 mol percent of the cationic lipid.

In one embodiment, the neutral lipid is present in a concentration ranging from 5 to 15 mol percent, from 7 to 13 mol percent, or from 9 to 11 mol percent. In one embodiment, the neutral lipid is present in a concentration of about 9.5, 10 or 10.5 mol percent.

In one embodiment, the steroid is present in a concentration ranging from 30 to 50 mol percent, from 35 to 45 mol percent or from 38 to 43 mol percent. In one embodiment, the steroid is present in a concentration of about 40, 41, 42, 43, 44, 45 or 46 mol percent.

In one embodiment, the LNP comprises from 1 to 10 mol percent, from 1 to 5 mol percent, or from 1 to 2.5 mol percent of the polymer conjugated lipid.

In one embodiment, the LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and the RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure.

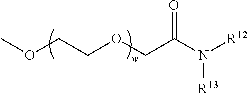

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In one embodiment, R¹² and R¹³ are each independently straight, saturated alkyl chains containing from 12 to 16 carbon atoms. In one embodiment, w has a mean value ranging from 40 to 55. In one embodiment, the average w is about 45. In one embodiment, R¹² and R¹³ are each independently a straight, saturated alkyl chain containing about 14 carbon atoms, and w has a mean value of about 45.

In one embodiment, the pegylated lipid is DMG-PEG 2000, e.g., having the following structure:

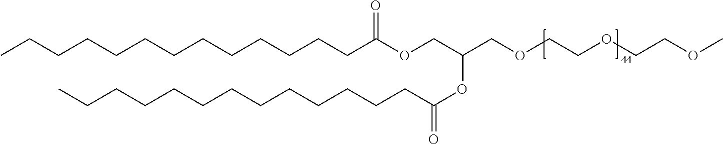

In some embodiments, the cationic lipid component of the LNPs has the structure of Formula (III):

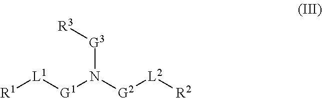

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

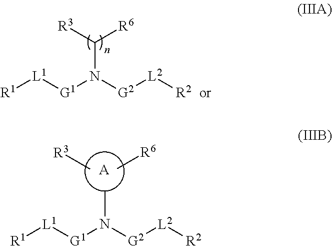

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

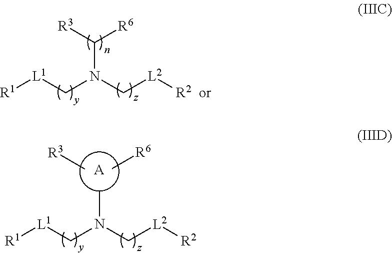

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

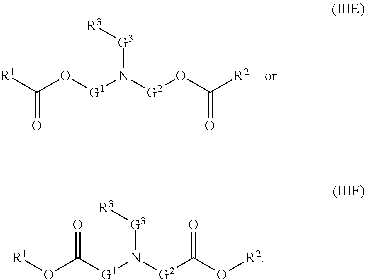

In some of the foregoing embodiments of Formula (III), the lipid has one of the following structures (IIIG), (IIIH), (IIII), or (IIIJ):

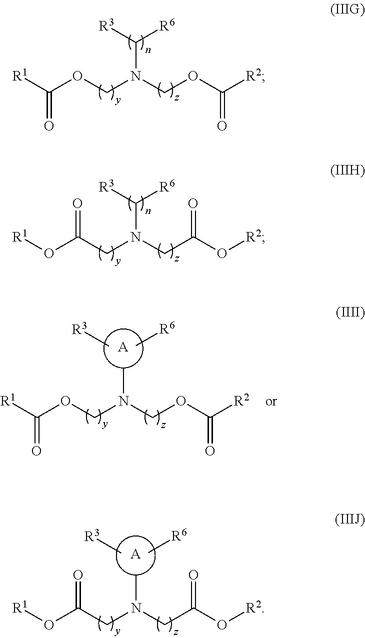

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

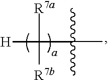

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

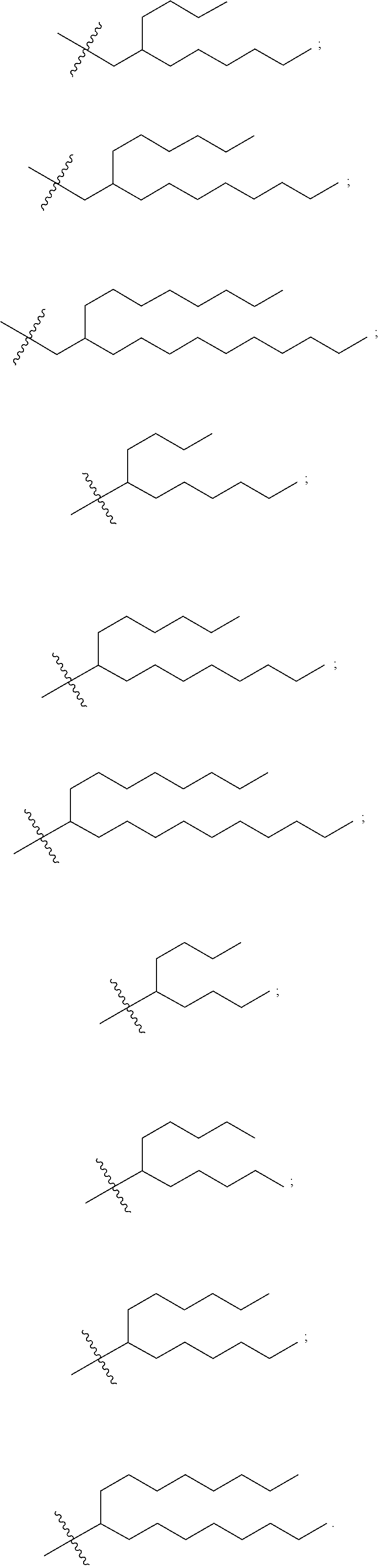

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

### Representative Compounds of Formula (III).

| No. | Structure |
|---|---|
| III-1 | 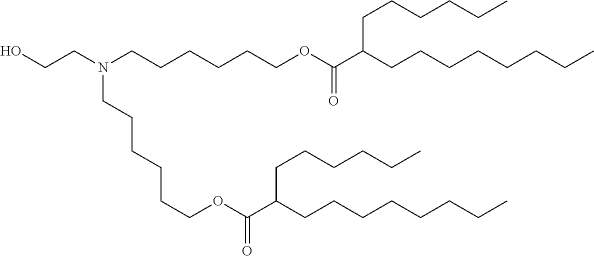 [SMILES: CCCCCCCCC(CCCCCC)C(=O)OCCCCCCN(CCO)CCCCCCOC(=O)C(CCCCCC)CCCCCCCC] |
| III-2 | 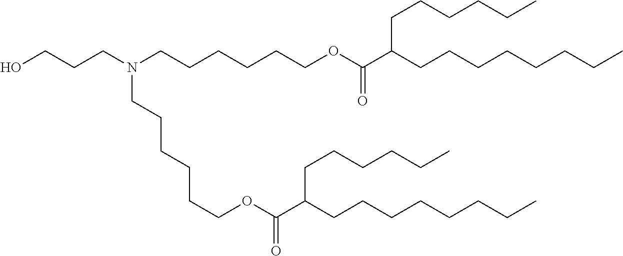 [SMILES: CCCCCCCCC(CCCCCC)C(=O)OCCCCCCN(CCCO)CCCCCCOC(=O)C(CCCCCC)CCCCCCCC] |
| III-3 | 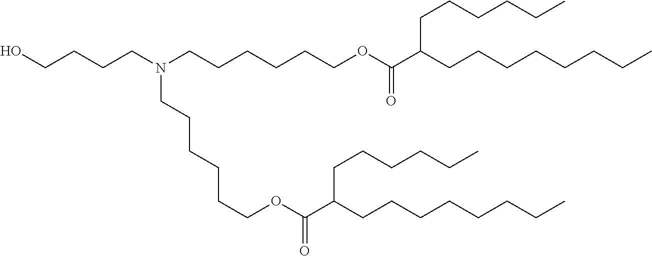 [SMILES: CCCCCCCCC(CCCCCC)C(=O)OCCCCCCN(CCCCO)CCCCCCOC(=O)C(CCCCCC)CCCCCCCC] |
| III-4 | 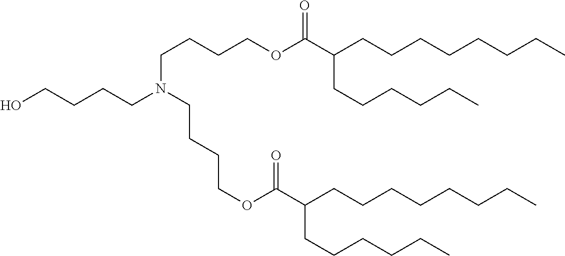 [SMILES: CCCCCCCCC(CCCCCC)C(=O)OCCCCN(CCCCO)CCCCOC(=O)C(CCCCCC)CCCCCCCC] |
| III-5 | 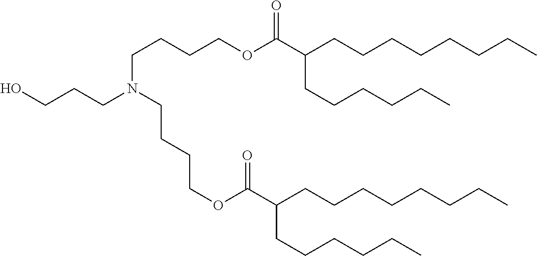 [SMILES: CCCCCCCCC(CCCCCC)C(=O)OCCCCN(CCCO)CCCCOC(=O)C(CCCCCC)CCCCCCCC] |
| III-6 | 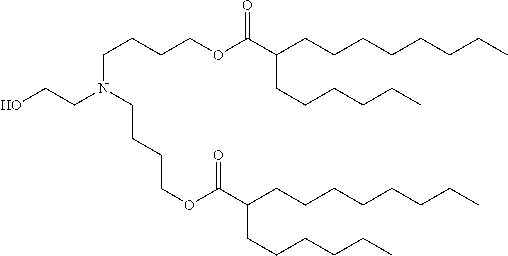 [SMILES: CCCCCCCCC(CCCCCC)C(=O)OCCCCN(CCO)CCCCOC(=O)C(CCCCCC)CCCCCCCC] |
| III-7 | 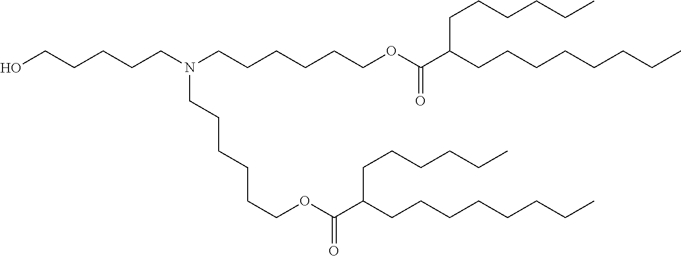 [SMILES: CCCCCCCCC(CCCCCC)C(=O)OCCCCCCN(CCCCCO)CCCCCCOC(=O)C(CCCCCC)CCCCCCCC] |
| III-8 | 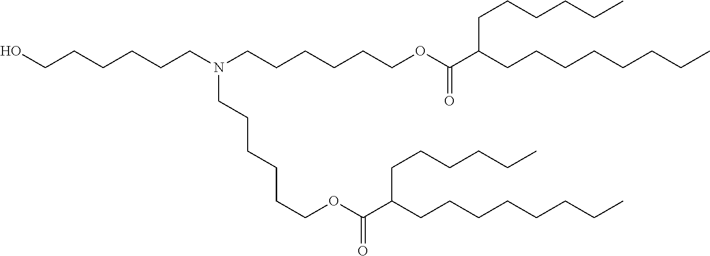 [SMILES: CCCCCCCCC(CCCCCC)C(=O)OCCCCCCN(CCCCCCO)CCCCCCOC(=O)C(CCCCCC)CCCCCCCC] |
| III-9 | 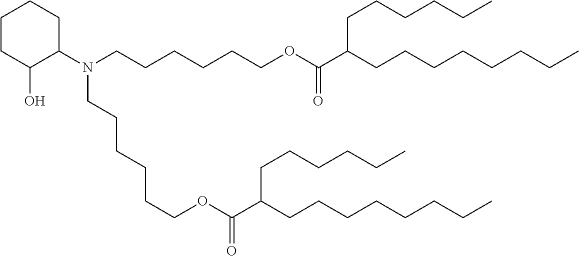 [SMILES: CCCCCCCCC(CCCCCC)C(=O)OCCCCCCN(CCCCCCOC(=O)C(CCCCCC)CCCCCCCC)C1CCCCC1O] |
| III-10 | 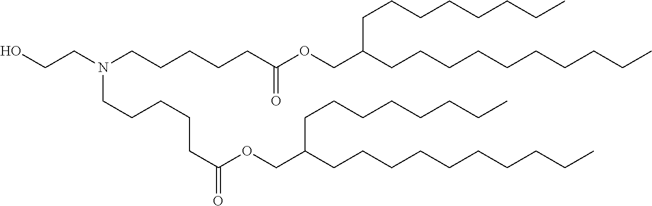 [SMILES: CCCCCCCCCCC(CCCCCCCC)COC(=O)CCCCCN(CCO)CCCCCC(=O)OCC(CCCCCCCC)CCCCCCCCCC] |
| III-11 | 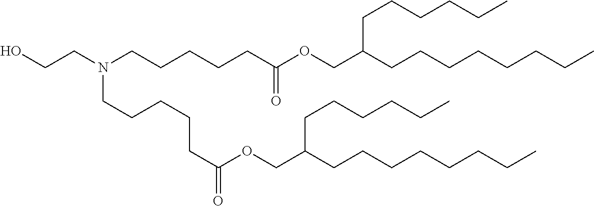 [SMILES: CCCCCCCCC(CCCCCC)COC(=O)CCCCCN(CCO)CCCCCC(=O)OCC(CCCCCC)CCCCCCCC] |
| III-12 | 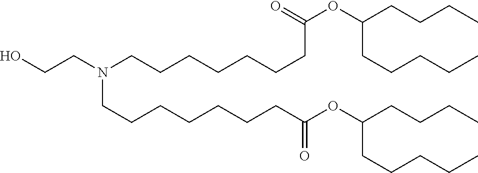 [SMILES: CCCCCC(CCCC)OC(=O)CCCCCCCN(CCO)CCCCCCCC(=O)OC(CCCC)CCCCC] |
| III-13 | 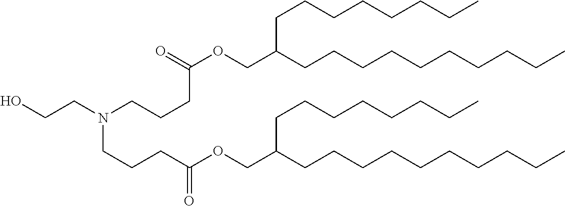 [SMILES: CCCCCCCCCCC(CCCCCCCC)COC(=O)CCCN(CCO)CCCC(=O)OCC(CCCCCCCC)CCCCCCCCCC] |
| III-14 | 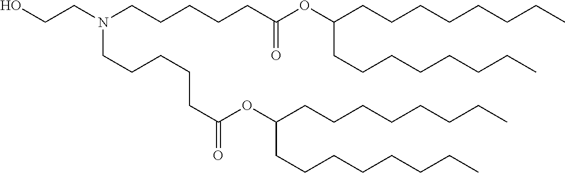 [SMILES: CCCCCCCCC(CCCCCCCC)OC(=O)CCCCCN(CCO)CCCCCC(=O)OC(CCCCCCCC)CCCCCCCC] |
| III-15 | 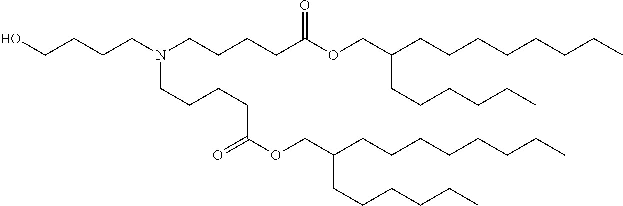 [SMILES: CCCCCCCCC(CCCCCC)COC(=O)CCCCN(CCCCO)CCCCC(=O)OCC(CCCCCC)CCCCCCCC] |
| III-16 | 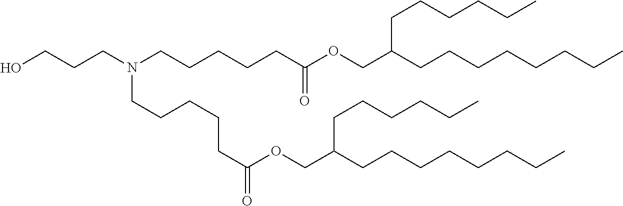 [SMILES: CCCCCCCCC(CCCCCC)COC(=O)CCCCCN(CCCO)CCCCCC(=O)OCC(CCCCCC)CCCCCCCC] |
| III-17 | 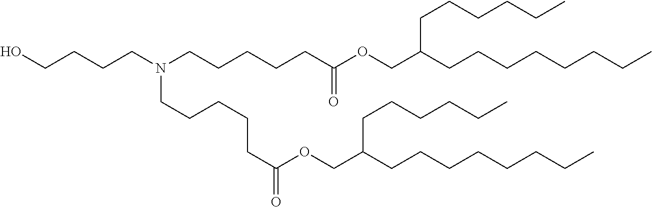 [SMILES: CCCCCCCCC(CCCCCC)COC(=O)CCCCCN(CCCCO)CCCCCC(=O)OCC(CCCCCC)CCCCCCCC] |
| III-18 | 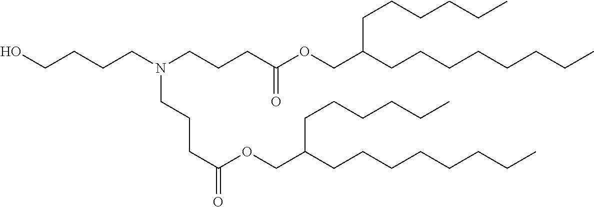 [SMILES: CCCCCCCCC(CCCCCC)COC(=O)CCCN(CCCCO)CCCC(=O)OCC(CCCCCC)CCCCCCCC] |
| III-19 | 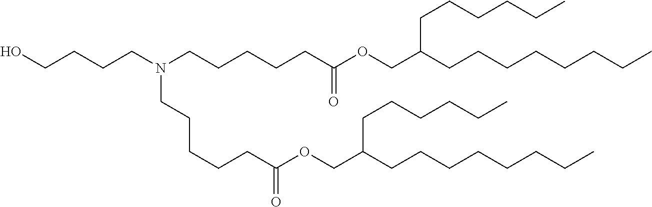 [SMILES: CCCCCCCCC(CCCCCC)COC(=O)CCCCCN(CCCCO)CCCCCC(=O)OCC(CCCCCC)CCCCCCCC] |
| III-20 | 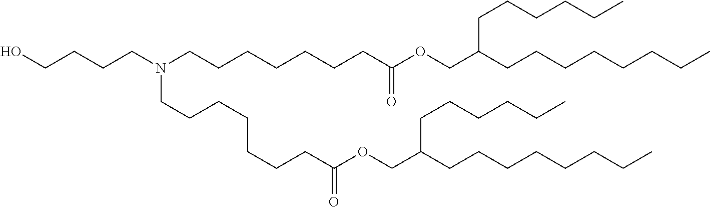 [SMILES: CCCCCCCCC(CCCCCC)COC(=O)CCCCCCCN(CCCCO)CCCCCCCC(=O)OCC(CCCCCC)CCCCCCCC] |
| III-21 | 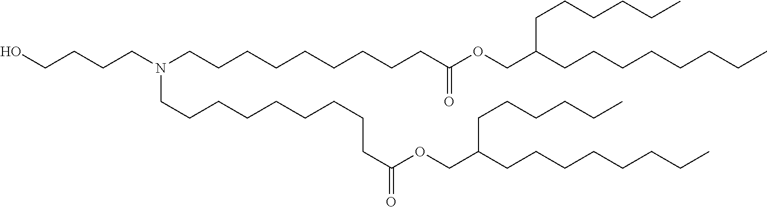 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 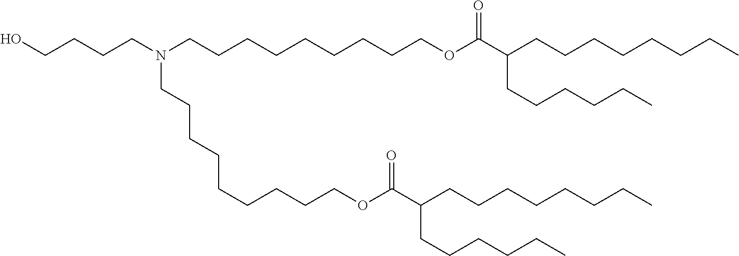 [SMILES: CCCCCCCCC(CCCCCC)C(=O)OCCCCCCCCCN(CCCCO)CCCCCCCCCOC(=O)C(CCCCCC)CCCCCCCC] |
| III-23 | 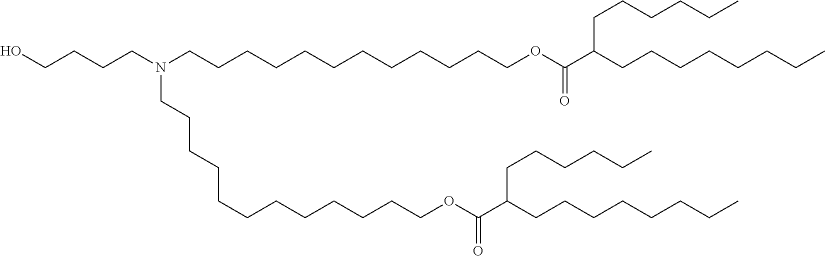 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 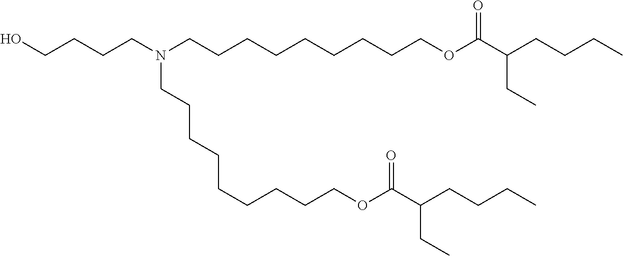 [SMILES: CCCCC(CC)C(=O)OCCCCCCCCCN(CCCCO)CCCCCCCCCOC(=O)C(CC)CCCC] |
| III-25 | 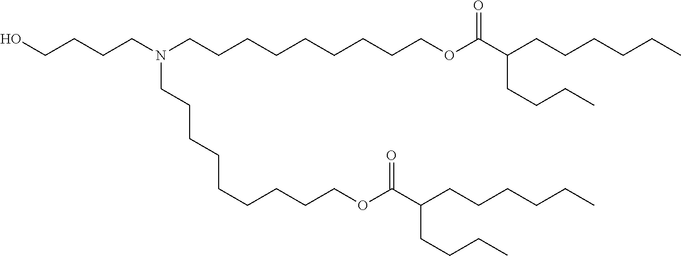 [SMILES: CCCCCCC(CCCC)C(=O)OCCCCCCCCCN(CCCCO)CCCCCCCCCOC(=O)C(CCCC)CCCCCC] |
| III-26 | 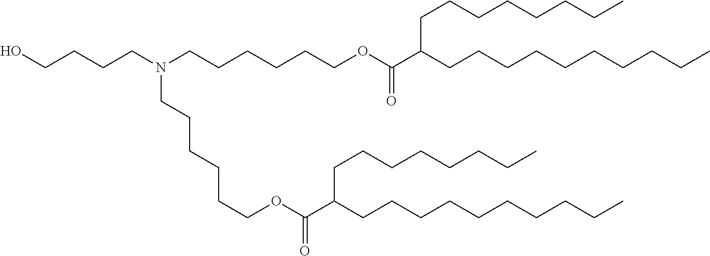 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 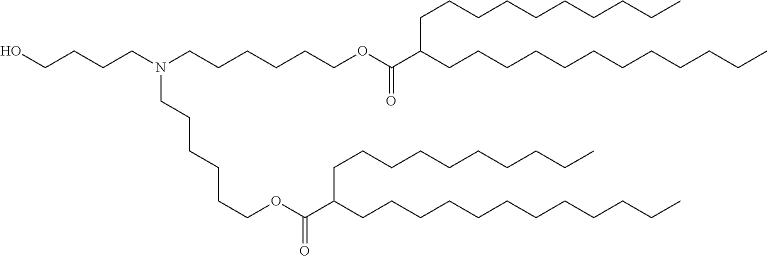 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 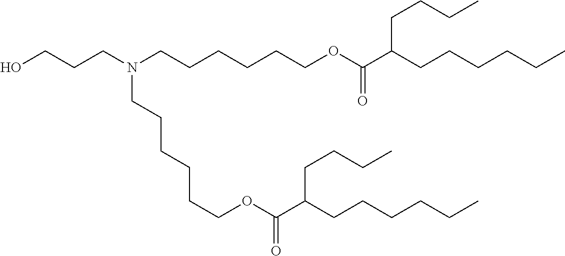 [SMILES: CCCCCCC(CCCC)C(=O)OCCCCCCN(CCCO)CCCCCCOC(=O)C(CCCC)CCCCCC] |
| III-29 | 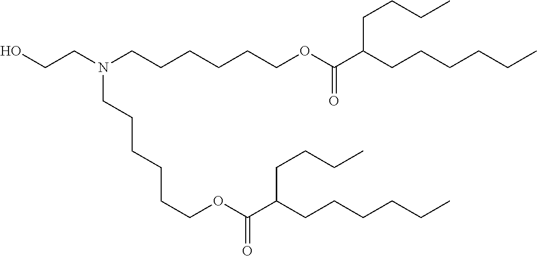 [SMILES: CCCCCCC(CCCC)C(=O)OCCCCCCN(CCO)CCCCCCOC(=O)C(CCCC)CCCCCC] |
| III-30 | 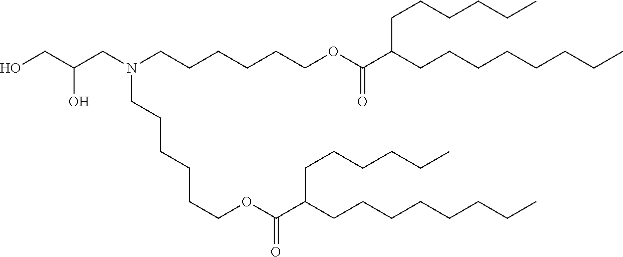 [SMILES: CCCCCCCCC(CCCCCC)C(=O)OCCCCCCN(CCCCCCOC(=O)C(CCCCCC)CCCCCCCC)CC(O)CO] |
| III-31 | 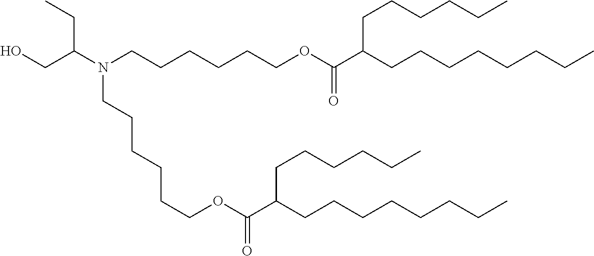 [SMILES: CCCCCCCCC(CCCCCC)C(=O)OCCCCCCN(CCCCCCOC(=O)C(CCCCCC)CCCCCCCC)C(CC)CO] |
| III-32 | 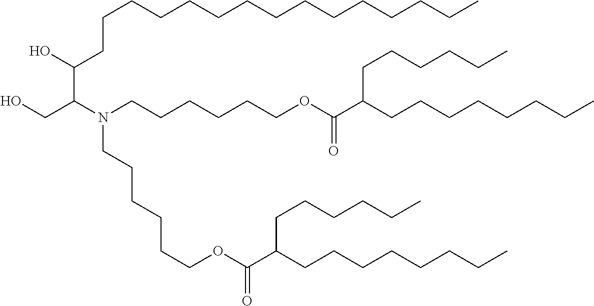 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 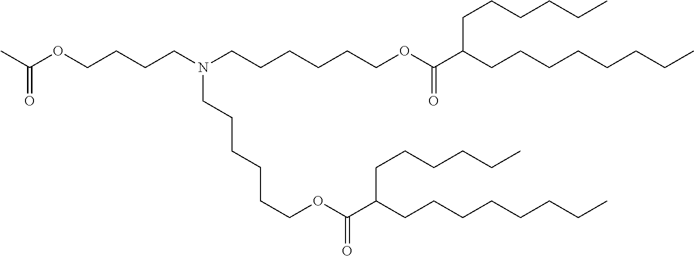 [SMILES: CCCCCCCCC(CCCCCC)C(=O)OCCCCCCN(CCCCCCOC(=O)C(CCCCCC)CCCCCCCC)CCCCOC(C)=O] |
| III-34 | 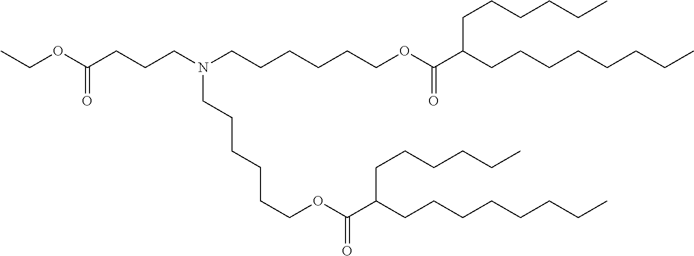 [SMILES: CCCCCCCCC(CCCCCC)C(=O)OCCCCCCN(CCCCCCOC(=O)C(CCCCCC)CCCCCCCC)CCCC(=O)OCC] |
| III-35 | 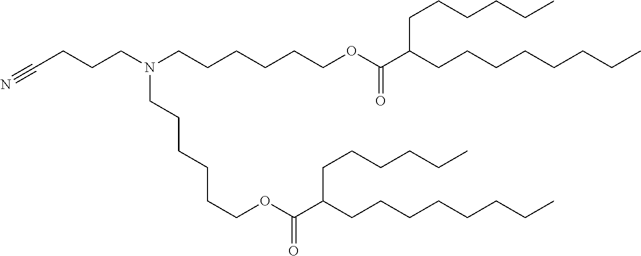 [SMILES: CCCCCCCCC(CCCCCC)C(=O)OCCCCCCN(CCCC#N)CCCCCCOC(=O)C(CCCCCC)CCCCCCCC] |
| III-36 | 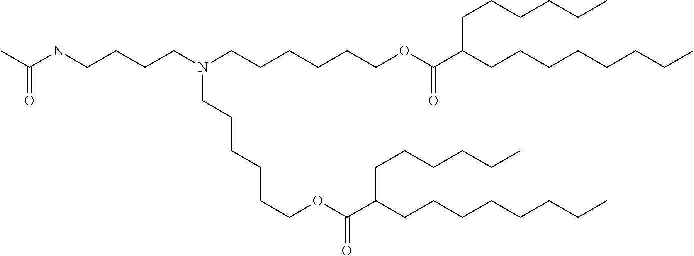 [SMILES: CCCCCCCCC(CCCCCC)C(=O)OCCCCCCN(CCCCCCOC(=O)C(CCCCCC)CCCCCCCC)CCCCNC(C)=O] |

In some embodiments, the LNP comprises a lipid of Formula (III), RNA, a neutral lipid, a steroid and a pegylated lipid. In some embodiments, the lipid of Formula (III) is compound III-3. In some embodiments, the neutral lipid is DSPC. In some embodiments, the steroid is cholesterol. In some embodiments, the pegylated lipid is ALC-0159.

In some embodiments, the cationic lipid is present in the LNP in an amount from about 40 to about 50 mole percent. In one embodiment, the neutral lipid is present in the LNP in an amount from about 5 to about 15 mole percent. In one embodiment, the steroid is present in the LNP in an amount from about 35 to about 45 mole percent. In one embodiment, the pegylated lipid is present in the LNP in an amount from about 1 to about 10 mole percent.

In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent.

In various different embodiments, the cationic lipid has one of the structures set forth in the table below.

| No. | Structure |
|---|---|
| A | 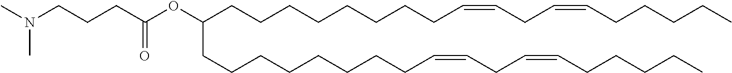 [SMILES: CCCCC/C=C\C/C=C\CCCCCCCCC(CCCCCCCC/C=C\C/C=C\CCCCC)OC(=O)CCCN(C)C] |
| B | 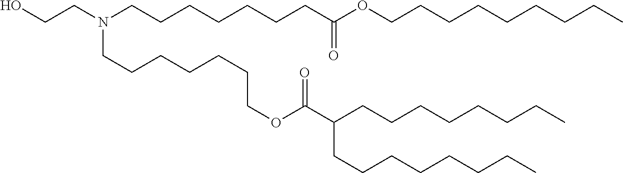 [SMILES: CCCCCCCCCOC(=O)CCCCCCCN(CCO)CCCCCCCOC(=O)C(CCCCCCCC)CCCCCCCC] |
| C | 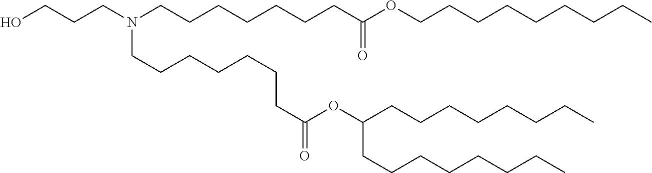 [SMILES: CCCCCCCCCOC(=O)CCCCCCCN(CCCO)CCCCCCCC(=O)OC(CCCCCCCC)CCCCCCCC] |
| D | 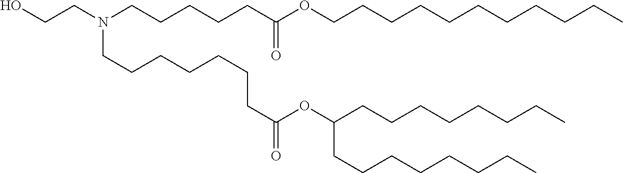 [SMILES: CCCCCCCCCCCOC(=O)CCCCCN(CCO)CCCCCCCC(=O)OC(CCCCCCCC)CCCCCCCC] |
| E | 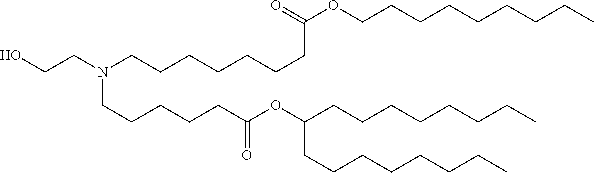 [SMILES: CCCCCCCCCOC(=O)CCCCCCCN(CCO)CCCCCC(=O)OC(CCCCCCCC)CCCCCCCC] |
| F | 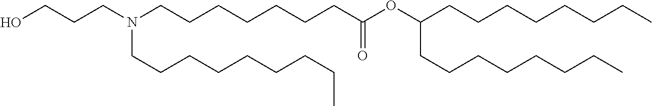 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC. In some embodiments, the steroid is cholesterol. In some embodiments, the pegylated lipid is DMG-PEG 2000.

In one embodiment, the LNP comprises a cationic lipid that is an ionizable lipid-like material (lipidoid). In one embodiment, the cationic lipid has the following structure:

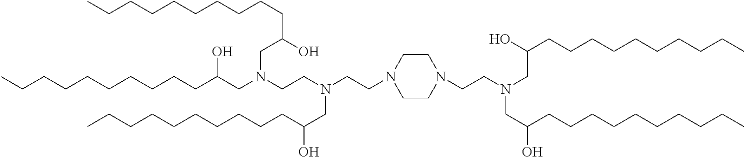 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The N/P value is preferably at least about 4. In some embodiments, the N/P value ranges from 4 to 20, 4 to 12, 4 to 10, 4 to 8, or 5 to 7. In one embodiment, the N/P value is about 6.

LNP described herein may have an average diameter that in one embodiment ranges from about 30 nm to about 200 nm, or from about 60 nm to about 120 nm.

### RNA Targeting

Some aspects of the disclosure involve the targeted delivery of the rRNA disclosed herein (e.g., RNA encoding vaccine antigens and/or immunostimulants).

In one embodiment, the disclosure involves targeting lung. Targeting lung is in particular preferred if the RNA administered is RNA encoding vaccine antigen. RNA may be delivered to lung, for example, by administering the RNA which may be formulated as particles as described herein, e.g., lipid particles, by inhalation.

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2, or about 1.6:2 to about 1:2, or about 1.6:2 to about 1.1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

Immunostimulants may be provided to a subject by administering to the subject RNA encoding an immunostimulant in a formulation for preferential delivery of RNA to liver or liver tissue. The delivery of RNA to such target organ or tissue is preferred, in particular, if it is desired to express large amounts of the immunostimulant and/or if systemic presence of the immunostimulant, in particular in significant amounts, is desired or required.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

Examples of suitable immunostimulants for targeting liver are cytokines involved in T cell proliferation and/or maintenance. Examples of suitable cytokines include IL2 or IL7, fragments and variants thereof, and fusion proteins of these cytokines, fragments and variants, such as extended-PK cytokines.

In another embodiment, RNA encoding an immunostimulant may be administered in a formulation for preferential delivery of RNA to the lymphatic system, in particular secondary lymphoid organs, more specifically spleen. The delivery of an immunostimulant to such target tissue is preferred, in particular, if presence of the immunostimulant in this organ or tissue is desired (e.g., for inducing an immune response, in particular in case immunostimulants such as cytokines are required during T-cell priming or for activation of resident immune cells), while it is not desired that the immunostimulant is present systemically, in particular in significant amounts (e.g., because the immunostimulant has systemic toxicity).

Examples of suitable immunostimulants are cytokines involved in T cell priming. Examples of suitable cytokines include IL12, IL15, IFN-α, or IFN-β, fragments and variants thereof, and fusion proteins of these cytokines, fragments and variants, such as extended-PK cytokines.

### Methods for Producing a Protein

The present invention also provides a method for producing a protein of interest in a cell comprising the steps of:
(a) obtaining the RNA replicon according to the invention, which comprises an open reading frame encoding the protein of interest, and
(b) inoculating the RNA replicon into the cell.

In various embodiments of the method, the RNA replicon is as defined above for the RNA replicon of the invention, as long as the RNA replicon comprises an open reading frame encoding the protein of interest, optionally an open reading frame encoding functional non-structural protein, and can be replicated by the functional non-structural protein, wherein the rRNA may comprising at least one modified nucleotide and one or more point mutations in a regulatory sequence that restores or improves a function of the modified rRNA.

The cell into which one or more nucleic molecule can be inoculated can be referred to as “host cell”. According to the invention, the term “host cell” refers to any cell which can be transformed or transfected with an exogenous nucleic acid molecule. The term “cell” preferably is an intact cell, i.e. a cell with an intact membrane that has not released its normal intracellular components such as enzymes, organelles, or genetic material. An intact cell preferably is a viable cell, i.e. a living cell capable of carrying out its normal metabolic functions. The term “host cell” comprises, according to the invention, prokaryotic (e.g. E. coli) or eukaryotic cells (e.g. human and animal cells, plant cells, yeast cells and insect cells). Particular preference is given to mammalian cells such as cells from humans, mice, hamsters, pigs, domesticated animals including horses, cows, sheep and goats, as well as primates. The cells may be derived from a multiplicity of tissue types and comprise primary cells and cell lines. Specific examples include keratinocytes, peripheral blood leukocytes, bone marrow stem cells and embryonic stem cells. In other embodiments, the host cell is an antigen-presenting cell, in particular a dendritic cell, a monocyte or a macrophage. A nucleic acid may be present in the host cell in a single or in several copies and, in one embodiment is expressed in the host cell.

The cell may be a prokaryotic cell or a eukaryotic cell. Prokaryotic cells are suitable herein e.g. for propagation of DNA according to the invention, and eukaryotic cells are suitable herein e.g. for expression of the open reading frame of the replicon.

In the method of the present invention, any of the RNA replicon according to the invention, or the kit according to the invention, or the pharmaceutical composition according to the invention, can be used. RNA can be used in the form of a pharmaceutical composition, or as naked RNA e.g. for electroporation.

In the method for producing a protein in a cell according to the present invention, the cell may be an antigen presenting cell, and the method may be used for expressing the RNA encoding the antigen. To this end, the invention may involve introduction of RNA encoding antigen into antigen presenting cells such as dendritic cells. For transfection of antigen presenting cells such as dendritic cells a pharmaceutical composition comprising RNA encoding the antigen may be used.

In one embodiment, a method for producing a protein in a cell is an in vitro method. In one embodiment, a method for production of a protein in a cell does not comprise the removal of a cell from a human or animal subject by surgery or therapy.

In this embodiment, the cell inoculated according to the invention may be administered to a subject so as to produce the protein in the subject and to provide the subject with the protein. The cell may be autologous, syngeneic, allogeneic or heterologous with respect to the subject.

In other embodiments, the cell in a method for producing a protein in a cell may be present in a subject, such as a patient. In these embodiments, the method for producing a protein in a cell is an in vivo method which comprises administration of RNA molecules to the subject.

In this respect, the invention also provides a method for producing a protein of interest in a subject comprising the steps of:
(a) obtaining the RNA replicon according to the invention, which comprises an open reading frame encoding the protein of interest, and
(b) administering the RNA replicon to the subject.

In various embodiments of the method, the RNA replicon is as defined above for the RNA replicon of the invention, as long as the RNA replicon comprises an open reading frame encoding the protein of interest, optionally an open reading frame encoding functional non-structural protein, and can be replicated by the functional non-structural protein, wherein the rRNA may comprising at least one modified nucleotide and one or more point mutations in a regulatory sequence that restores or improves a function of the modified rRNA.

Any of the RNA replicon according to the invention, or the kit according to the invention, or the pharmaceutical composition according to the invention can be used in the method for producing a protein in a subject according to the invention. For example, in the method of the invention, RNA can be used in the format of a pharmaceutical composition, e.g. as described herein, or as naked RNA.

In view of the capacity to be administered to a subject, each of the RNA replicon according to the invention, or the kit according to the invention, or the pharmaceutical composition according to the invention, may be referred to as “medicament”, or the like. The present invention foresees that the RNA replicon, the kit, the pharmaceutical composition of the present invention is provided for use as a medicament. The medicament can be used to treat a subject. By “treat” is meant to administer a compound or composition or other entity as described herein to a subject. The term includes methods for treatment of the human or animal body by therapy.

The above described medicament does typically not comprise a DNA, and is thus associated with additional safety features compared to DNA vaccines described in the prior art (e.g. WO 2008/119827 A1).

An alternative medical use according to the present invention comprises a method for producing a protein in a cell according to the present invention, wherein the cell may be an antigen presenting cell such as a dendritic cell, followed by the introduction of said cell to a subject. For example, RNA encoding a pharmaceutically active protein, such as an antigen, may be introduced (transfected) into antigen-presenting cells ex vivo, e.g. antigen-presenting cells taken from a subject, and the antigen-presenting cells, optionally clonally propagated ex vivo, may be reintroduced into the same or a different subject. Transfected cells may be reintroduced into the subject using any means known in the art.

The medicament according to the present invention may be administered to a subject in need thereof. The medicament of the present invention can be used in prophylactic as well as in therapeutic methods of treatment of a subject.

The medicament according to the invention is administered in an effective amount. An “effective amount” concerns an amount that is sufficient, alone or together with other doses, to cause a reaction or a desired effect. In the case of treatment of a certain disease or a certain condition in a subject, the desired effect is the inhibition of disease progression. This includes the deceleration of disease progression, in particular the interruption of disease progression. The desired effect in the treatment of a disease or a condition can also be a delay of disease outbreak or the inhibition of disease outbreak.

The effective amount will depend on the condition being treated, the severity of the disease, the individual parameters of the patient, including age, physiological condition, size and weight, duration of the treatment, type of accompanying therapy (if any), the specific mode of administration and other factors.

### Vaccination

The term “immunization” or “vaccination” generally refers to a process of treating a subject for therapeutic or prophylactic reasons. A treatment, particularly a prophylactic treatment, is or comprises preferably a treatment aiming to induce or enhance an immune response of a subject, e.g. against one or more antigens. If, according to the present invention, it is desired to induce or enhance an immune response by using rRNA as described herein, the immune response may be triggered or enhanced by the rRNA. In one embodiment, the invention provides a prophylactic treatment which is or comprises preferably the vaccination of a subject. An embodiment of the present invention wherein the replicon encodes, as a protein of interest, a pharmaceutically active peptide or protein which is an immunologically active compound or an antigen is particularly useful for vaccination.

RNA has been previously described for vaccination against foreign agents including pathogens or cancer (reviewed recently by Ulmer et al., 2012, Vaccine 30:4414-4418). In contrast to common approaches in the prior art, the replicon according to the present invention is a particularly suitable element for efficient vaccination because of the ability to be replicated by functional alphavirus non-structural protein as described herein. The vaccination according to the present invention can be used for example for induction of an immune response to weakly immunogenic proteins. In the case of the RNA vaccines according to the invention, the protein antigen is never exposed to serum antibodies, but is produced by transfected cells themselves after translation of the RNA. Therefore, anaphylaxis should not be a problem. The invention therefore permits the repeated immunization of a patient without risk of allergic reactions.

In methods involving vaccination according to the present invention, the medicament of the present invention is administered to a subject, in particular if treating a subject having a disease involving the antigen or at risk of falling ill with the disease involving the antigen is desired.

In methods involving vaccination according to the present invention, the protein of interest encoded by the replicon according to the present invention codes for example for a bacterial antigen, against which an immune response is to be directed, or for a viral antigen, against which an immune response is to be directed, or for a cancer antigen, against which an immune response is to be directed, or for an antigen of a unicellular organism, against which an immune response is to be directed. The efficacy of vaccination can be assessed by known standard methods such as by measurement of antigen-specific IgG antibodies from the organism. In methods involving allergen-specific immunotherapy according to the present invention, the protein of interest encoded by the replicon according to the present invention codes for an antigen relevant to an allergy. Allergen-specific immunotherapy (also known as hypo-sensitization) is defined as the administration of preferably increasing doses of an allergen vaccine to an organism with one or more allergies, in order to achieve a state in which the symptoms that are associated with a subsequent exposure to the causative allergen are alleviated. The efficacy of an allergen-specific immunotherapy can be assessed by known standard methods such as by measurement of allergen-specific IgG and IgE antibodies from the organism.

The medicament of the present invention can be administered to a subject, e.g. for treatment of the subject, including vaccination of the subject.

The term “subject” relates to vertebrates, particularly mammals. For example, mammals in the context of the present invention are humans, non-human primates, domesticated mammals such as dogs, cats, sheep, cattle, goats, pigs, horses etc., laboratory animals such as mice, rats, rabbits, guinea pigs, etc. as well as animals in captivity such as animals of zoos. The term “subject” also relates to non-mammalian vertebrates such as birds (particularly domesticated birds such as chicken, ducks, geese, turkeys) and to fish (particularly farmed fish, e.g., salmon or catfish). The term “animal” as used herein also includes humans.

The administration to domesticated animals such as dogs, cats, rabbits, guinea pigs, hamsters, sheep, cattle, goats, pigs, horses, chicken, ducks, geese, turkeys, or wild animals, e.g., foxes, is preferred in some embodiments. For example, a prophylactic vaccination according to the present invention may be suitable to vaccinate an animal population, e.g. in the farming industry, or a wild animal population. Other animal populations in captivity, such as pets, or animals of zoos, may be vaccinated.

### Mode of Administration

The medicament according to the present invention can be applied to a subject in any suitable route.

For example, the medicament may be administered systemically, for example intravenously (i.v.), subcutaneously (s.c.), intradermally (i.d.) or by inhalation.

In one embodiment, the medicament according to the present invention is administered to muscle tissue, such as skeletal muscle, or skin, e.g. subcutaneously. It is generally understood that transfer of RNA into the skin or muscles leads to high and sustained local expression, paralleled by a strong induction of humoral and cellular immune responses (Johansson et al., 2012, PLoS. One. 7:e29732; Geall et al., 2012, Proc. Natl. Acad. Sci. U.S.A 109:14604-14609).

Alternatives to administration to muscle tissue or skin include, but are not limited to: intradermal, intranasal, intraocular, intraperitoneal, intravenous, interstitial, buccal, transdermal, or sublingual administration. Intradermal and intramuscular administration are two preferred routes.

Administration can be achieved in various ways. In one embodiment, the medicament according to the present invention is administered by injection. In a preferred embodiment, injection is via a needle. Needle-free injection may be used as an alternative.

The present invention is described in detail and is illustrated by the figures and examples, which are used only for illustration purposes and are not meant to be limiting. Owing to the description and the examples, further embodiments which are likewise included in the invention are accessible to the skilled worker.

### DESCRIPTION OF THE DRAWINGS

FIG. 1. Vector design (not drawn to scale). (1A) self-amplifying RNA (saRNA or rRNA) constructed from an alphaviral genome. saRNA is a bicistronic RNA with a 5′-open reading frame (ORF) encoding for the alphaviral RNA-dependent RNA polymerase (replicase) and a 3′-open reading frame (ORF) encoding for a gene of interest. ORFs are marked with the AUG-start codon. The coding regions of the saRNA is flanked by viral 5′- and 3′-untranslated regions (vUTR). Further, it contains regulatory regions made of conserved sequence elements (CSEs) needed for RNA replication (CSE1 & 2 comprising the genomic plus strand-promoter (5′-replication recognition sequence RRS), CSE4 the core genomic negative strand promotor and CSE3 the subgenomic promotor). Of note, both, 5′- and 3′-CSEs cooperate to initiate negative strand synthesis. The start codon for the ORF of the replicase is within the 5′-regulatory region. (1B) A trans-amplifying RNA system used for directed evolution. The alphaviral replicase is encoded by a non-replicating mRNA having non-viral 5′ and 3′ UTRs, and a long poly-A tail (pA). This mRNA is co-transfected together with a trans-replicon encoding cell surface expressed CD90.1 used as selectable surface marker. The trans-replicon was constructed such that the 5′ RRS was lacking AUGs and the coding sequences were inserted downstream starting with their own explicit AUG. The construct allows nucleotides to change without affecting the selectable marker gene.

FIG. 2. Trans-replicon template and RNA synthesis. (2A) A CD90.1 encoding trans-replicon is inserted into a plasmid which is PCR-amplified by a forward primer that attaches a T7-RNA polymerase promotor to the 5′ end, and a reverse primer that binds at the 3′ end of the CD90.1 ORF overlapping with the stop-codon. The reverse primer attaches a minimal 3′ CSE and a short poly-A (30 adenosines) to the 3′ end. The resulting PCR product serves as template for T7 polymerase driven RNA in vitro transcription (IVT), using either regular nucleotides ATP, CTP, GTP and UTP, or ATP, CTP, GTP and N1-methyl-pseudo-UTP. (2B) Replicase mRNA synthesis. A plasmid encoding a T7 promotor, a 5′ UTR, the replicase ORF, a 3′ UTR and a long poly A tail (100 A) is linearized with a restriction enzyme at the end of the poly-A tail. Using regular nucleotides, this linearized plasmid is in vitro transcribed to a non-modified (UTP containing) mRNA that expresses the replicase.

FIG. 3. In vitro evolution procedure. Templated by a PCR product the initial (N=0) trans-replicon coding for CD90.1 (TR-CD90.1) is in vitro transcribed (IVT) to unmodified RNA (containing uridine (U), or to modified RNA (containing N1-methypseudouridine (1mY (or m1ψ)). Each of the in vitro transcribed RNAs is electroporated together with replicase-encoding mRNA into cells. The next day, CD90.1 surface expression is assessed by flow cytometry. Initially, the expression by a replicable RNA or trans-replicon (TR) comprising U is much higher than by a TR comprising 1mY instead, as sketched in the idealized bar chart. The ratio of total expression by modified versus unmodified RNA (1mY-index) is far below 1. Next, modified TR transfected cells that express CD90.1 are enriched by magnetic assisted cell sorting (MACS), lysed and cellular TR-copies are converted to cDNA and a novel PCR product by one-step RT-PCR using TR-specific primers. This cycle N+1 PCR-product is then again used to synthesize U and 1mY TR-RNA for a novel electroporation of cells. This procedure was repeated N times until a robust and reproducible increase of the 1mY-index with increasing numbers of cycles is observed as sketched in the idealized bar chart, and was observed as demonstrated herein.

FIG. 4. TR-CD90.1 acquires increased compatibility with 1mY modification after 11 cycles of in vitro evolution. After 11 cycles of in vitro evolution, the resulting cell-extracted and reverse transcribed TR was in vitro transcribed using increasing amounts of 1mY instead of UTP. These increasingly modified TRs were electroporated into K562 cells together with replicase-encoding mRNA. (4A) The next day CD90.1 expression was assessed by flow cytometry. (4B and 4B-1; 4B-1 represents repeated experiments with statistical analysis) The 1mY-index was calculated as ratio of CD90.1 expression by modified against the unmodified TR.

FIG. 5. Accumulation of point mutations with the 5′ CSE-region after 11 cycles of in vitro evolution. After 11 cycles of in vitro evolution the resulting cell-extracted and reverse transcribed TR was PCR amplified and sequenced by NGS. Mutated TR transcripts were ranked according to their frequency. The mutations indicated here were the four most frequent, or present in the four most frequent transcripts, and selected for further analysis. All other mutations were found with maximal frequencies below 0.85% and listed here. The numbers equal the nucleotide position with 1 being the first in vitro transcribed G.

FIG. 6. Point mutations identified within the 5′ CSE increase the replication and expression of modified trans-replicons. (6A) K562 cell were electroporated with 2.6 μg unmodified mRNA coding for SFV-Replicase and 0.03 μg modified (1mY) or unmodified (UTP) VEEV-NTRs coding for CD90.1. The NTRs were either non-mutant (WT) or having the identified in vitro evolved mutations within the 5′ CSE as indicated. (6B) The next day, CD90.1 expression resulting from the NTRs was assessed by flow cytometry. Transfection rates are indicated by the numbers above the marker bars within the histograms. (6C and 6C-1; 6C-1 represents repeated experiments with statistical analysis) The 1mY index was calculated based on the flow cytometry data. The index quantifies the ratio of expression by modified against unmodified NTRs.

FIG. 7. A single 5′ nucleotide exchange originating from the VEEV-TC-83 strain improves expression of modified and unmodified trans-replicons in response to SFV-replicase. (7A) K562 cell were electroporated with 2.6 μg unmodified mRNA for SFV-Replicase and 0.03 μg modified (1mY) or unmodified (UTP) VEEV-NTRs coding for CD90.1. The NTRs were either non-mutant (WT) or having the TC-83 specific 5′ mutation (exchange of G to a at position 4, counted from the 5′-terminal G of the vectors. (7B) The next day, CD90.1 expression resulting from the NTR was assessed by flow cytometry. Transfection rates are indicated by the numbers above the marker within the histograms. (7C, 7C-1; 7C-1 represents repeated experiments with statistical analysis) The 1mY index was calculated based on the flow cytometry data. The index quantifies the ratio of expression by modified against unmodified NTRs.

FIG. 8. The TC-83 specific 5′ mutation cooperates with point mutations identified by in vitro evolution to further increase expression of modified trans-replicons amplified by the SFV-replicase. (8A) K562 cell were electroporated with 2.6 μg unmodified mRNA for SFV-Replicase and 0.03 μg modified (1mY) or unmodified (UTP) VEEV-NTRs coding for CD90.1. The NTRs were either non-mutant (WT), carried the 5′ mutation of VEEV-TC-83 (TC-83), or in addition the identified in vitro evolved mutations within the 5′ CSE as indicated. (8B) The next day, CD90.1 expression resulting from the NTR was assessed by flow cytometry. Transfection rates are indicated by the numbers above the marker bars within the histograms. (8C, 8C-1; 8C-1 represents repeated experiments with statistical analysis) The 1mY index was calculated based on the flow cytometry data. The index quantifies the ratio of expression by modified against unmodified NTRs.

FIG. 9. Trans-amplifying RNA expression is greatly increased in human fibroblasts when replicase mRNA and in vitro evolved trans-replicon RNA are both modified. (9A) Primary human foreskin fibroblasts were plated at 50,000 cells/well into 12-well-plates and lipofected the next day with 4 μl MessengerMax and 1 μg total RNA per well (0.9 μg SFV-replicase mRNA and 0.1 μg trans-replicon). The SFV-replicase mRNA (REPL) or the NTR were used in different variations as indicated. The next day, CD90.1 expression resulting from the NTR was assessed by flow cytometry. Transfection rates are indicated by the numbers above the marker within the histograms. (9B) The transfection rates displayed as bar graph. Abbreviations: UTP: unmodified RNA; 1mY: modified RNA; WT: non-mutant; -dsRNA: removal of double stranded RNA; TC-83: G4A-mutant; 3xMut=in vitro evolved mutants at position 67, 245, 248).

FIG. 10. The VEEV-TC-83 5′ CSE increases the expression of a chimeric VEEV/SFV modified self-amplifying RNA. (10A) To construct a chimeric VEEV/SFV saRNA coding for luciferase, the 5′ CSE of an SFV-saRNA was removed, including the part coding for the replicase N-terminus. This part was replaced by sequences corresponding to the deleted replicase N-terminus, preceded by a VEEV 5′ CSE depleted for start codons. A second similar construct carried the VEEV-TC-83 specific 5′-mutation G4A. RNA of both constructs was lipofected using MessengerMax into (10B) K562 cells or (10C) primary human foreskin fibroblasts (HFF). Luciferase expression was measured over 72 hours and the area under the curve (AUC) was calculated from line graphs to estimate the total expression. (10D, 10E) K562 or HFF cells were lipofected in 12-well-plates with comparable VEEV/SFV saRNA as in FIGS. 10B and 10C, but encoding GFP. The cells were harvested after 24 hours and the transfection rate was assessed by flow cytometry.

FIG. 11. FIG. 11A depicts the sequence of SEQ ID NO: 1, which is the 5′ regulatory region of VEEV Trinidad donkey strain (Accession No. L01442) (positions 45 to 47 are the start codon of nsP1). FIG. 11B depicts the sequence of SEQ ID NO: 2 used in the examples, which is a modified version of the 5′ regulatory region of VEEV Trinidad donkey strain (Accession No. L01442). For example, the G residue at position 1 was added for efficient in vitro transcription using the T7 RNA polymerase. Positions 280 to 282 in SEQ ID NO: 2 are the start codon for the encoded reporter gene in the examples. In an embodiment, this start codon also can be used for an encoded replicase.

FIG. 12. FIGS. 12A-12D depicts that fully modified, 1mY-adapted replicative RNA induces a robust immune response in mice. Mice were immunized to evaluate whether a 100% modified rRNA can be used for vaccination and whether the performance is comparable to a non-modified rRNA. Humoral immunity was assessed by (12A) ELISA to quantify HA-specific IgG antibodies or (12B) virus neutralization titer (VNT). T cell immunity was assessed by IFN-γ ELISpot with splenocytes stimulated with HA peptides specific either for (12C) MHC-I or (12D) MHC-II. Abbreviations: HA: Hemagglutinin of Influenza A virus; UTP: unmodified RNA; 1mY: modified RNA; WT: non-mutant; TC-83: G4A-mutant; 3xMut: in vitro evolved mutants at position 67, 245, 248; ELISA: enzyme-linked immunosorbent assay; ELISpot: enzyme-linked immune absorbent spot; IFN-γ: Interferon gamma; MHC: major histocompatibility complex.

FIG. 13. FIGS. 13A-13C depicts that TR-3xmut acquires increased compatibility with 1mY modification when subjected to 18 cycles of in vitro evolution together with the EEEV-replicase and 15 cycles together with the VEEV-replicase. (13A) K562 cells were electroporated with 2.6 μg mRNA encoding SFV-replicase or VEEV-replicase, and 54 ng of modified or unmodified TR transcribed from RT-PCR-products from extracted cellular RNA after the indicated rounds of the in vitro evolution process that resulted eventually in the identification of TR-3xmut. The next day the expression of encoded CD90.1 selection marker was assessed by flow cytometry and 1mY index was calculated as done before. The graphs show mean and SD of three independent experiments. Comparable to the procedure of panel A, K562 cells were electroporated with 1.3 μg mRNA encoding the replicases of either EEEV (13B) or VEEV (13C), and 5 ng or 15 ng of modified and unmodified TRs obtained from cell-derived cDNA after the indicated cycles of in vitro evolution Here, the in vitro evolution started using TR-3xmut as the starting TR. Again, 1mY index was determined the day after transfection.

### EXAMPLES

### Material and Methods

The following materials and methods were used in the examples.

Cloning of plasmids, In vitro transcription, RNA purification: Plasmids were cloned using standard technology. The details on the cloning of individual plasmids used in the examples of this invention are as described in Example 1 of WO 2017/162265 A1 and WO 2017/162266 A1. Briefly, one plasmid encodes the replicase of a Venezuelan Equine Encephalitis virus Trinidad donkey strain (VEEV; GenBank Accession No. L01442) inserted into the backbone of a non-replicating mRNA, and a second plasmid encodes a replicable RNA or trans-replicon RNA TR(ΔATG-RRS ΔSGP) expressing murine CD90.1. Point mutations that were identified during the in vitro evolution procedure were inserted into the 5′ ΔATG-RRS by gene synthesis and subcloning. As appropriate, the CD90.1 ORF was exchanged for GFP. Vector versions denominated “TC-83” differ from the others by a single nucleotide exchange near the 5′-end which was identified in the attenuated vaccine strain TC-83 of VEEV. The first 4 nucleotides at 5′-end of the synthetic RNA are thereby either 5′GAUG (Trinidad donkey) or 5′-GAUA (TC-83).

For in vitro transcription, these plasmids were linearized by restriction digestion downstream to the poly-A or amplified by PCR, and served as templates for T7 RNA-polymerase. RNA synthesis and purification were performed as previously described (Holtkamp et al., 2006, Blood 108:4009-4017; Kuhn et al., 2010, Gene Ther. 17:961-971). Double-stranded (ds) RNA depletion was done by cellulose purification as previously described (Baiersdörfer et al., 2019, Mol Ther Nucleic Acids 15:26-35). As required, UTP was exchanged by N1-methylpseudo-UTP at different ratios as indicated.

The quality of purified in vitro transcribed RNA was assessed by spectrophotometry, and analyzed on the 5200 Fragment Analyzer (Advanced Analytical). All RNA transfected into cells in the examples was in vitro transcribed RNA (IVT-RNA).

Cell culture: All growth media, antibiotics and other supplements were supplied by Life Technologies/Gibco, except when stated otherwise. Fetal calf serum (FCS) was purchased from Sigma-Aldrich. Human foreskin fibroblasts were obtained from System Bioscience (HFF, neonatal) and were cultivated in minimum essential media (MEM) containing 15% FCS, 1% non-essential amino acids, 1 mM sodium pyruvate at 37° C. Cells were grown at 37° C. in humidified atmosphere equilibrated to 5% CO2. K-562 cells (ATCC; CCL-243) were grown in RPMI medium supplemented with 10% FCS.

RNA transfer into cells as performed by electroporation or by lipid-mediated transfection. For electroporation, RNA was resuspended in a final volume of 62.5 μl/mm cuvette gap size And was electroporated into cells at room temperature using a square-wave electroporation device (BTX ECM 830, Harvard Apparatus, Holliston, MA, USA). For the different cell types, the following settings were applied: HFF (500 V/cm, 1 pulse of 24 milliseconds (ms)); K562 (650 V/cm, 3 pulses of 8 ms and intervals of 400 ms). RNA lipofections were performed using Lipofectamine MessengerMAX following the manufacturer's instructions (Life Technologies, Darmstadt, Germany). Adherent cells were plated at approximately 20,000 cells/cm² growth area and transfected 24 hours later with a total amount of 250 ng/cm² RNA and 1 μl/cm² MessengerMAX. RNA species were mixed in RNAse-free Eppendorf tubes.

Luciferase Assays: To assess the expression of luciferase in transfected cells, transfected cells were plated in 96-well black microplates (Nunc, Langenselbold, Germany). The detection of firefly luciferase was performed with the Bright-Glo Luciferase Assay System according to the manufacturer's instructions. Bioluminescence was measured using a microplate luminescence reader Infinite M200 (Tecan Group, Männedorf, Switzerland). Luciferase activity determined at a given time point was plotted versus time, and area under the curve was calculated by the trapezoidal rule. Luciferase-negative cells were used to assess the background signal.

Magnetic-assisted cell sorting (MACS): CD90.1 expressing cells were first selected for viable cells (Dead cell removal kit, Miltenyi Biotec, Bergisch-Gladbach; Germany) and subsequently isolated from transfected cell bulks by MACS using anti-CD90.1 microbeads (Miltenyi Biotec, Bergisch-Gladbach; Germany) following the manufacturer's instruction.

Flow cytometry (CD90.1; GFP): For flow cytometry, cells were stained using a CD90.1/Thy-1.1-PE Clone OX-7 antibody (BD; Heidelberg; Germany), or when expressing GFP were left unstained. Marker expression was measured using the BD FACS Canto II flow cytometer. Data analysis was done using the companion FACS Diva software, or FlowJo software.

One-step reverse-transcription polymerase chain reaction (one-step RT-PCR): CD90.1 expressing cells were lysed using the iScript™ RT-qPCR sample preparation reagent (BioRad). Lysates were used for one-Step RT-PCR without prior RNA extraction using the SuperScript™ III One-Step RT-PCR System with Platinum™ Taq DNA Polymerase (Invitrogen).

Polymerase chain reaction (PCR): To generate a DNA template for in vitro transcription, the TR(ΔATG-RRS ΔSGP) was amplified by a specific forward primer including a T7-promoter, and a specific reverse primer binding to the 3′-end of CD90.1 and introducing a minimal 3′ CSE and a short poly-A of 30 adenosine residues. For PCR, the Q5® high-fidelity DNA-polymerase (NEB) was used according to the manufacturer's instruction.

Next-generation sequencing: To identify the accumulation of mutations upon 11 cycles of in vitro evolution, the cDNA corresponding to the 5′-RRS was amplified by PCR and resulting products sequenced on the Illumina MiSeq using MiSeq Reagent Kit v3 (Illumina).

### Example 1

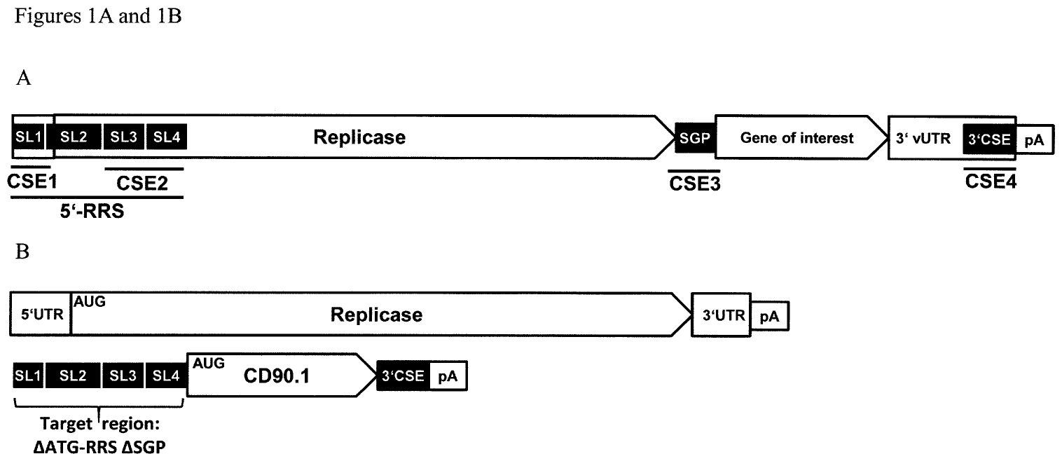

Self-amplifying RNA (saRNA) constructed from an alphaviral genome is characterized by the following indispensable structural domains and coding regions as described in FIG. 1A. Regarding the coding regions, saRNA, in its most common form, is a bicistronic RNA with two open reading frames (ORFs). The 5′-ORF encodes for the alphaviral non-structural polyprotein (nsP), consisting of 4 subunits with all enzymatic functions necessary for RNA-dependent RNA transcription. The nsP maturates stepwise and builds into a protein complex (so-called replicase) that is associated with cellular membranes where it forms spherules that serve as a compartment for RNA-amplification. The second ORF downstream of and separated from the replicase ORF by a subgenomic promoter (SGP) takes up the genetic information of genes of interest, very often antigens. A number of RNA-structural domains conserved between alphaviral species (conserved sequence elements; CSEs) govern interaction with replicase and thereby govern RNA replication. At the 5′ end, 2 CSEs are within a longer highly structured region (referred to as 5′-replication recognition sequence; RRS) that overlaps with the ORF of the replicase. This region is much longer and more structured than the other more downstream CSEs (CSE3=subgenomic promoter and CSE4=3′-terminal CSE). In addition, saRNA is 5′-capped and 3′-poly-adenylated. The cap is needed for replicase translation, but otherwise dispensable for replication. Besides enabling efficient translation of the replicase, the first approximately 15 adenosines of the poly-A tail are part of the 3′CSE.

For the procedure of in vitro evolution, we used a non-replicating mRNA to express the replicase as described in WO 2017/162265 A1 (FIG. 1B) and a short replicable RNA (rRNA) capable of being replicated in trans by the replicase (trans-replicon; TR). This TR is characterized by a 5′-RRS lacking start codons (ΔATG-RRS) and has no subgenomic promoter (SGP) (ΔSGP) as exemplarily described in WO 2017/162266 A1 followed downstream by the ORF for a detectable reporter surface protein (here: CD90.1) and at the 3′-end a minimal 3′CSE and a short poly-A of 30 adenosines.

### Example 2

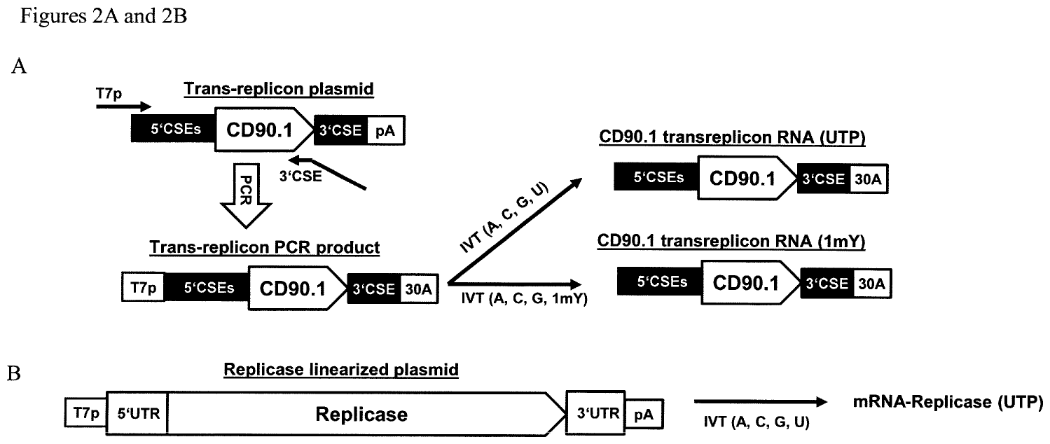

The CD90.1-TR, which is cloned into a plasmid, is PCR-amplified. The forward primer attaches a T7-RNA polymerase promotor to the 5′-end immediately upstream to the 5′-regulatory region. The reverse primer is complementary to the 3′ end of the CD90.1 ORF and has a non-binding overhang comprising a minimal 3′ CSE and a short poly-A (30 adenosines). The resulting PCR product is the template DNA for in vitro transcription using either regular nucleotides ATP, CTP, GTP, and either UTP or N1-methylpseudo-UTP (FIG. 2A). Replicase-encoding mRNA is synthesized using a plasmid having a T7 promotor immediately upstream of a non-alphaviral 5′ UTR. The plasmid is linearized with a restriction enzyme at the end of the poly-A tail. Using regular nucleotides, this linearized plasmid is in vitro transcribed to a non-modified (UTP containing) mRNA that expresses replicase (FIG. 2B).

### Example 3

The in vitro evolution described herein aims to select self- or trans-replicating RNA that conserves replication competence when it incorporates modified nucleotides, such as N1-methyl pseudouridine, during in vitro transcription. Due to the presence of such modified nucleotides, the RNA largely loses the capacity to stimulate innate immune receptors, thereby reducing the innate immune response to the in vitro transcribed RNA, and thus reduce side effects due to administration of the RNA. However, the presence of such modified nucleotides can also lead to a reduction in the ability of the RNA to replicate/be translated to produce the replicase and/or encoded gene of interest, e.g., antigen.

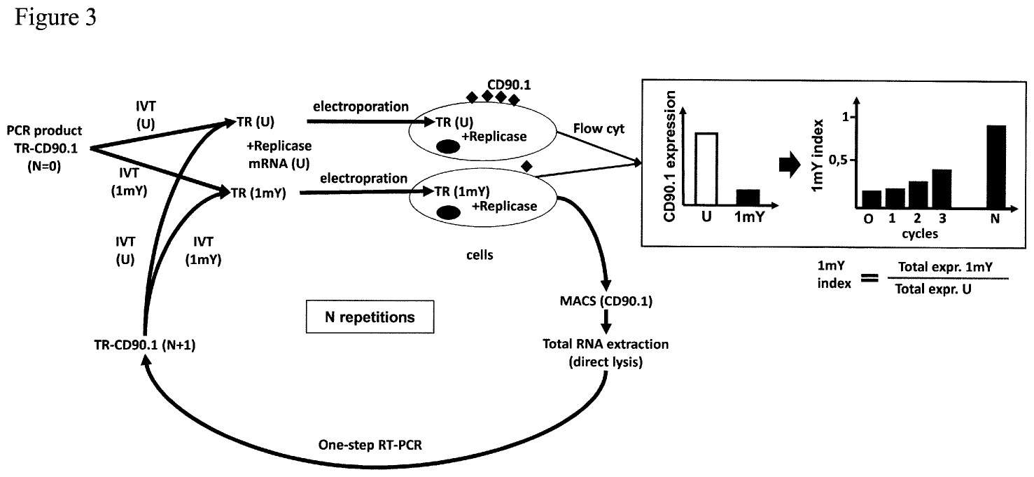

Due to its greater length, it was hypothesized that the 5′-regulatory region offers more options for sequence evolution than the short and simply structured 3′ CSE. Furthermore, the 5′ CSE is involved in negative strand synthesis, which is the first RNA transcription that uses the modified in vitro transcribed RNA as a template. The trans-replicon and replicase-mRNA vector, as well as the RNA synthesis procedure described above are suitable for a directed evolution strategy that specifically targets the 5′-regulatory region in a repetitive, cyclic procedure (FIG. 3). The procedure starts with a first PCR product spanning the trans-replicon coding for CD90.1 which is in vitro transcribed to a non-modified (U) and modified (1mY) RNA (cycle number N=0). Each RNA is electroporated together with a non-modified replicase-encoding mRNA into K562 cells. The next day, CD90.1 surface expression is assessed by flow cytometry. Initially, the expression by a TR (U) is much higher than by a TR (1mY), the ratio of total expression by modified versus non-modified RNA (1mY-index) is far below 1. Next, cells transfected with modified TR that express CD90.1 are enriched by magnetic-assisted cell sorting (MACS), lysed and cellular TR-copies are converted to cDNA and a novel PCR product by one-step RT-PCR using TR-specific primers. This cycle N+1 PCR-product is then again used to synthesize U and 1mY TR-RNA for electroporation into the cells. By repeating this procedure, it is expected that the 1mY-index will increase with each step.

### Example 4

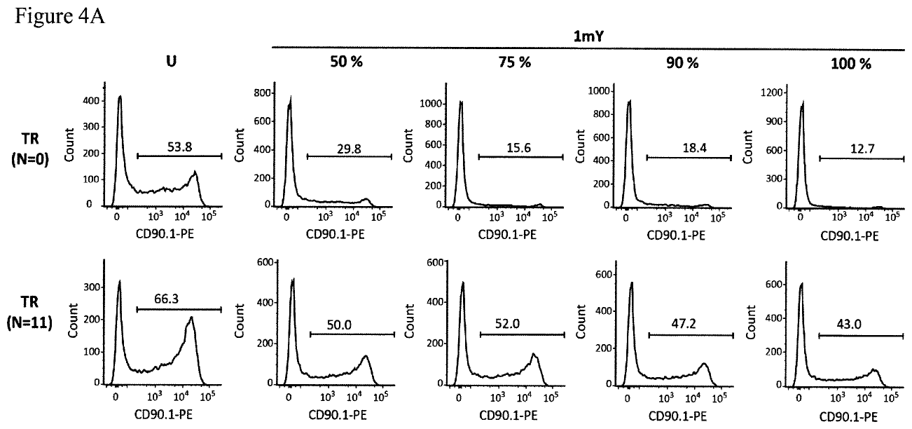

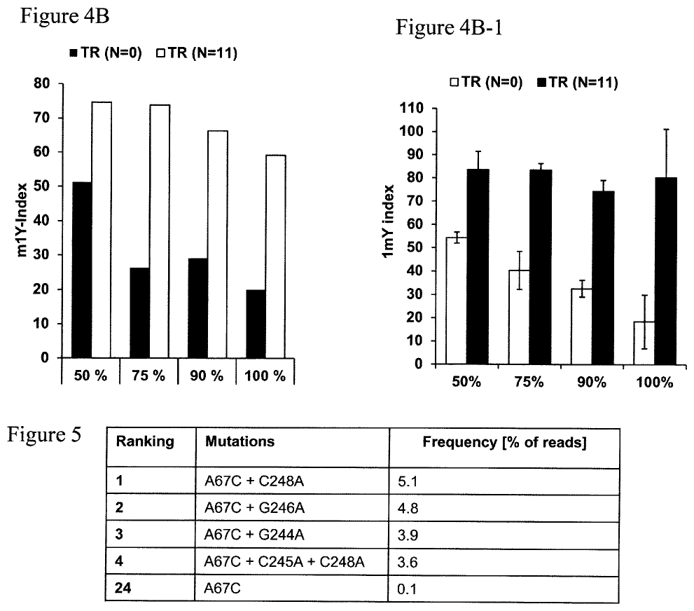

TR-CD90.1 acquires increased compatibility with 1mY modification after 11 cycles of in vitro evolution. The in vitro evolution procedure was performed until a robust and reproducible increase of the 1mY-index was observed. After 11 cycles, TR-RNA from the cells expressing CD90.1 was obtained and reverse-transcribed to produce a sufficient amount of PCR product for subsequent analysis. First, it was aimed to independently confirm from the in vitro evolution procedure that the TR had evolved to a trans-replicating RNA preserving replication upon increasing rates of modification (percentage of modified nucleotides). In vitro transcription of the TR resulting from 11 cycles of in vitro evolution (N=11), as well as the initial TR template (N=0) using increasing rates of 1mY instead of UTP (50 to 100%) was performed. The increasingly modified TRs were electroporated into K562 cells together with replicase-encoding mRNA. The next day, CD90.1 expression of the initial TR proved more sensitive towards increasing 1mY-modification as the TR after 11 cycles, as assessed by flow cytometry and visualized by an increased 1mY-index (FIG. 4). Since the 1mY index was increased for all percentage of 1mY we concluded that the initial sequence had indeed evolved and acquired a greater compatibility for the incorporation of 1mY.

### Example 5

Accumulation of point mutations with the 5‘ ’CSE-region after 11 cycles of in vitro evolution was demonstrated. Having confirmed in Example 4 that 11 cycles of in vitro evolution increased the compatibility for 1mY modification, it was decided to sequence the PCR-product and analyze whether specific mutations had accumulated with the 5′ regulatory region. Next generation sequence (NGS) analysis revealed a number of point mutants that were present with frequencies of several percent of the reads. A group of 4 sequences with related mutations were found in 17.4% of all reads, with clonal frequencies from 5.1% to 3.6%. These 4 clones had double or triple mutations, the first always being an exchange from A to C at position 67, the second or third being nucleotide exchanges at positions 244 to 248 (FIG. 5). The mutation A67C that was common to all was itself ranking at position 24. The 4 most frequent clones and the less frequent, but common mutation were selected for clonal analysis.

### Example 6

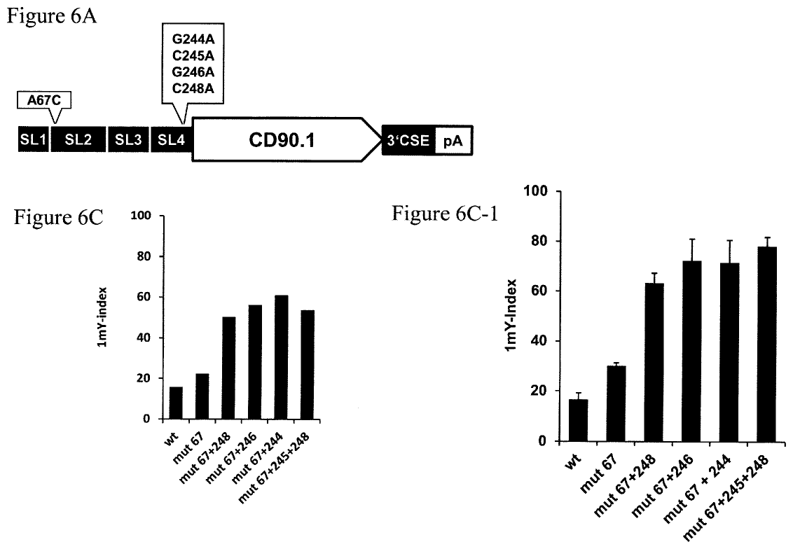

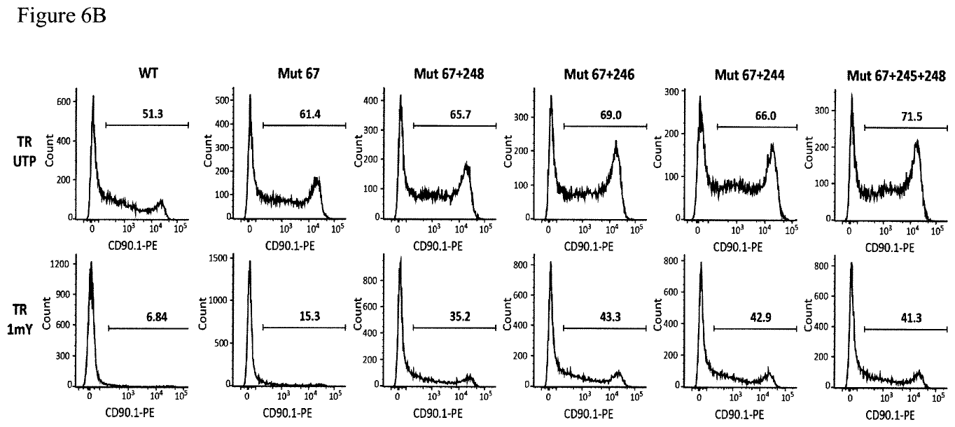

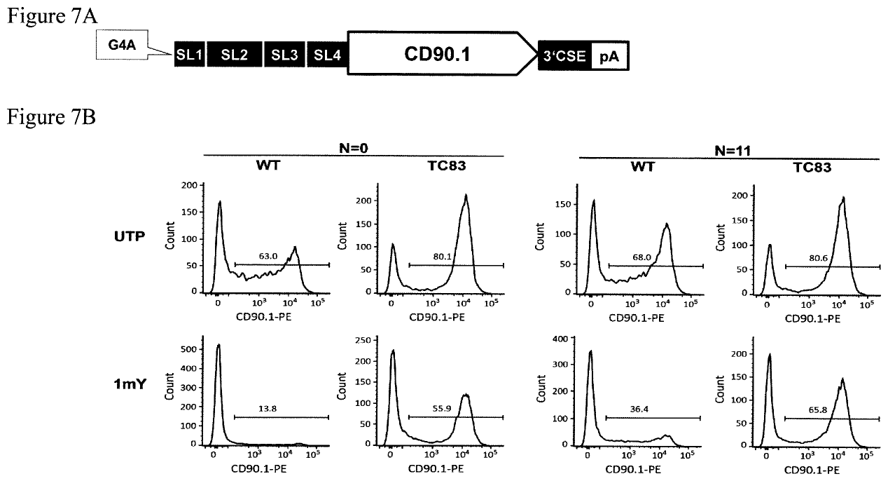

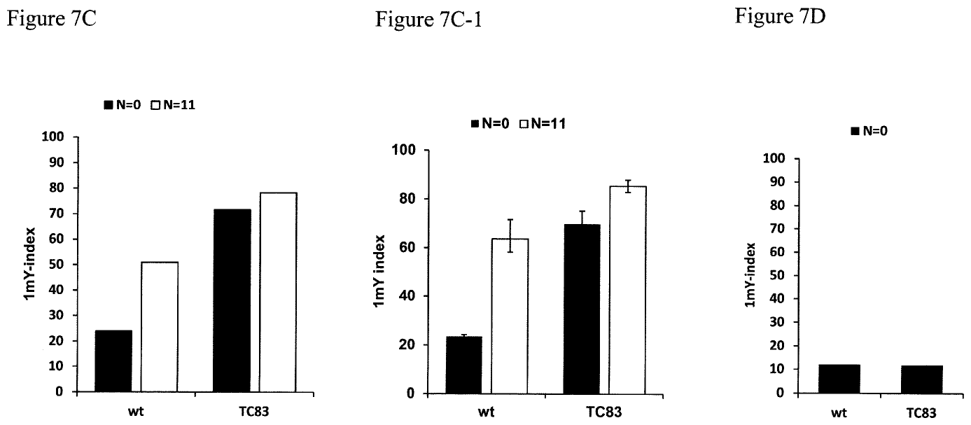

Point mutations identified within the 5′ CSE increase the replication and expression of modified trans-replicons. The identified mutated residues were inserted into the parental TR sequence and tested. Non-modified and modified RNA of all clones were constructed and co-transfected into K562 cells with the replicase-encoding mRNA. The next day, transfection rates and 1mY index were assessed by flow cytometry, which showed that all clones were more resistant towards modification by 1mY than the initial non-mutated TR (WT) (FIG. 6).

### Example 7

A single 5′ nucleotide exchange originating from the VEEV-TC-83 strain improves expression of modified and unmodified trans-replicons in response to SFV-replicase. The replicable RNA replicating in trans was engineered from the alphavirus Venezuelan equine encephalitis virus (VEEV), while the replicase-encoding mRNA originated from the alphavirus Semliki Forest virus (SFV). For VEEV, a vaccine strain was selected decades ago that displays an attenuated phenotype, the so-called TC-83 strain (Berge et al., 1961, American Journal of Epidemiology 73:209-218). TC-83 harbors several mutations compared to the virulent Trinidad donkey strain of VEEV. Very well characterized is a nucleotide exchange close to the 5′-end within the first CSE. This mutation alters the secondary structure of the 5′-terminal stem-loop of CSE1, and this correlated to an increased replication of the VEEV-TC-83 genomic RNA (Kulasegaran-Shylini et al., 2009, Virology 387:211-221). We observed that this 5′-mutation enhances the replication in trans in response to the SFV-replicase and increases the replication of a 1mY-modified replicable RNA. When then the TC-83 5′-mutation is present in the TRs after 11 cycles of in vitro evolution, it was observed that the 1mY index was slightly further increased (FIGS. 4B and 4C). Interestingly, in response to a VEEV-replicase-encoding mRNA, the 1mY-index of TRs remained unchanged upon the incorporation of the TC-83 mutation (FIG. 4D).

### Example 8

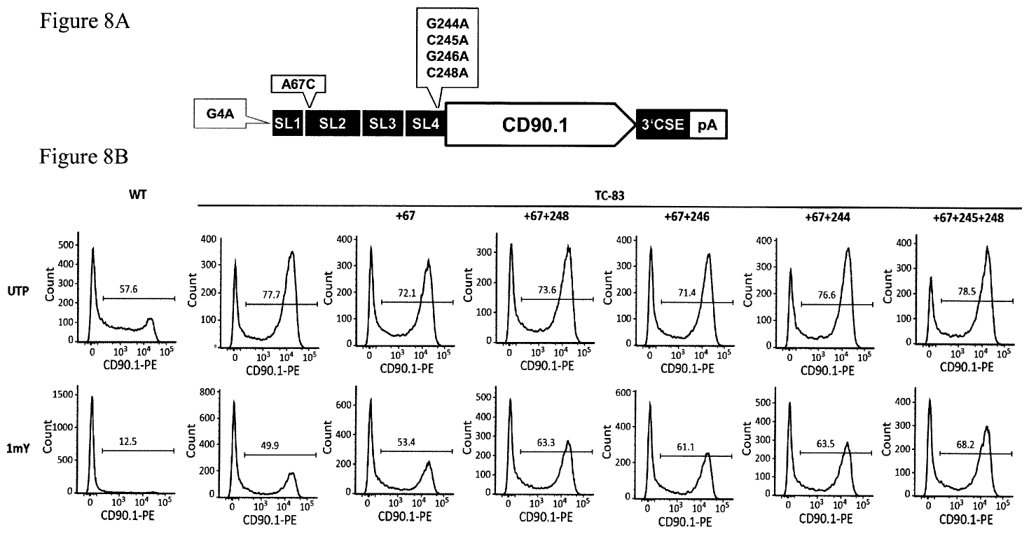

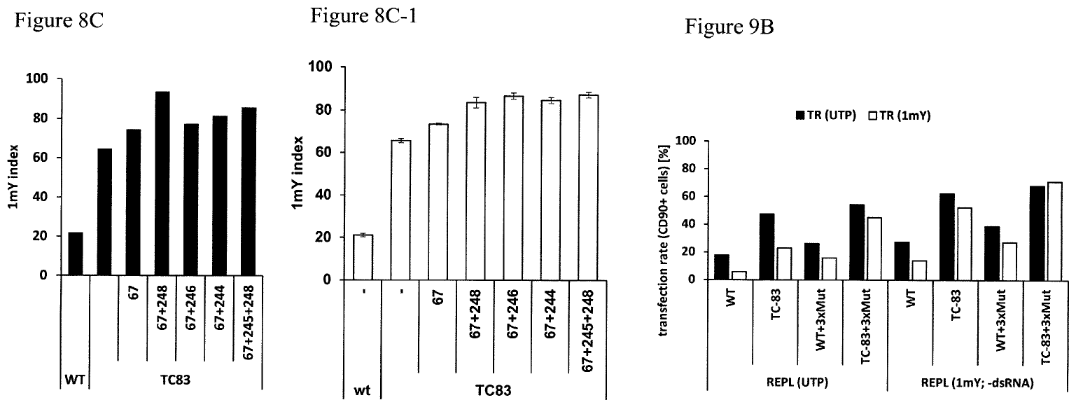

The TC-83-specific 5′ mutation cooperates with the point mutations created by in vitro evolution to further increase expression of modified trans-replicons amplified by the SFV-replicase. Next, it was assessed whether the TC-83 specific 5′-mutation would further increase the compatibility of the mutant TR-clones selected by in vitro evolution. Using PCR, clonal TRs with and without TC-83 mutation were generated, either as non-modified or modified RNA. K562 cells were co-electroporated with replicase-encoding mRNA and identical amounts of all of the clones. It was observed that, again, the TC-83 specific mutation alone increased the expression of modified TR, illustrated by a higher transfection rate and 1mY index compared to non-modified TR. Further, the in vitro-evolved point mutants were able to further increase transfection rates and 1mY indices (FIG. 8).

### Example 9

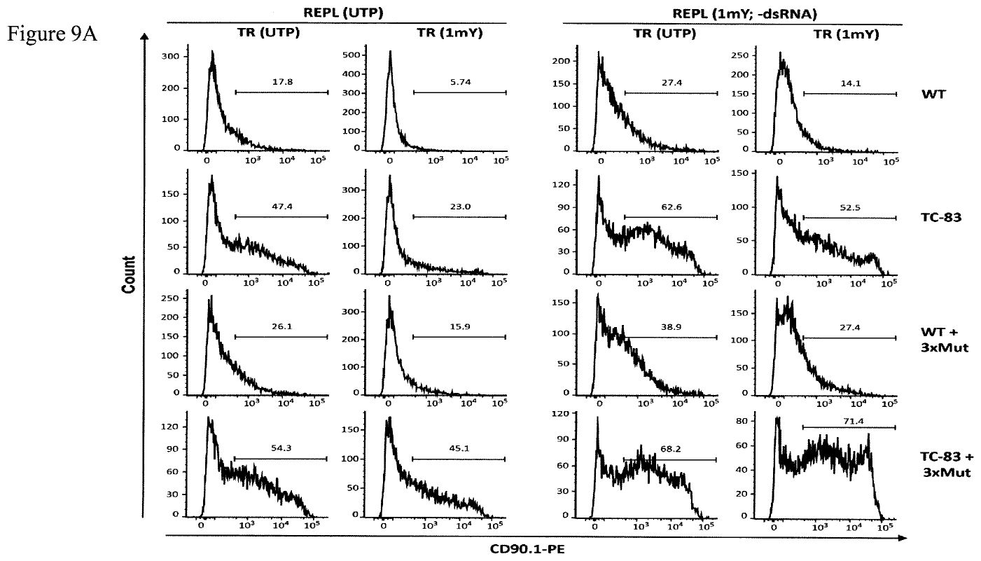

Trans-amplifying RNA expression is greatly increased in human fibroblasts when replicase and trans-replicon RNA are modified and carrying in vitro evolved mutations. The K562 cells that were used for the in vitro evolution enable a very efficient expression of self- and trans-replicating RNA molecules, which indicates that these cells have a weak antiviral response to the RNA. Although such a cellular system allows selecting for replicating RNA that is resistant to nucleotide modification, it is not suitable to study the expression of modified RNA in presence of strong cellular innate immune response triggered by pattern recognition receptors. Therefore, primary human foreskin fibroblasts were used to study the expression of the in vitro evolved TRs. In the first branch of the experiment, non-modified SFV-replicase mRNA, and non-modified or modified TRs were used to lipofect fibroblasts. The TRs were either of the initial non-mutant sequence (WT), a TC-83 variant of the initial sequence (TC-83), and the triple mutant clone identified after 10 cycles of in vitro evolution, either with TC-83 mutation (TC-83+3xMut), or without TC-83 mutation (WT+3xMut). In the second branch of the experiment, the same RNA combinations were co-lipofected, with the difference that the replicase-encoding mRNA was also modified by 1mY, and further purified to remove double-stranded RNA contaminants (Baiersdörfer et al., 2019, Mol Ther Nucleic Acids 15:26-35). When modified replicase and modified TRs were combined, the RNA transfection is theoretically the least immunogenic. It was found that the greatest transfection rates of fibroblasts were achieved within the low immunogenic branch and with the modified TC-83 variant of the triple mutant (FIG. 9). These data show that a non-immunogenic trans-replicating RNA that preserves replication and enables robust transgene expression was generated.

### Example 10

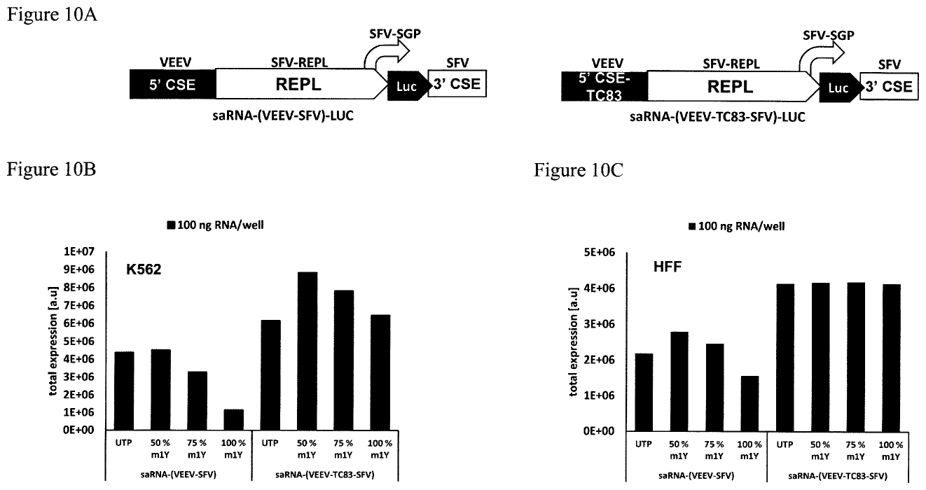

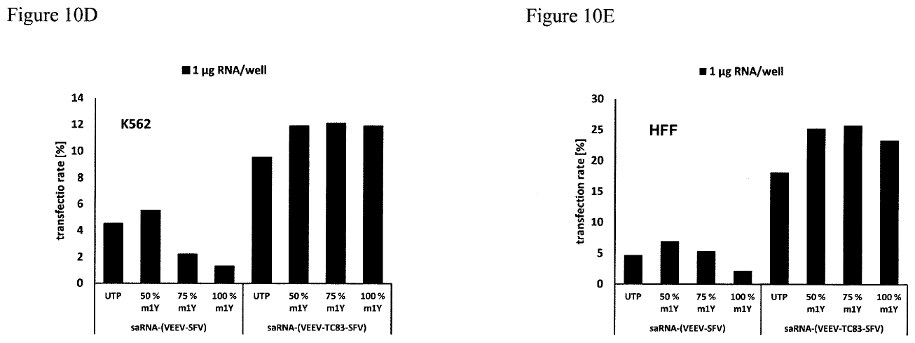

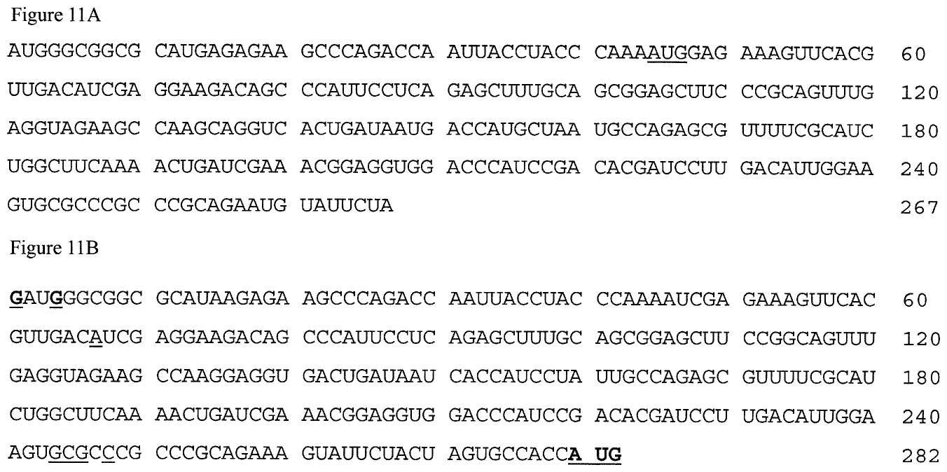

The VEEV-TC-83 5′ CSE increases the expression of a chimeric VEEV/SFV modified self-amplifying RNA. Having identified 5′-regulatory regions that enable RNA replication upon modification, it was aimed to construct a corresponding self-replicating RNA. First, the VEEV 5′ regulatory region was used to construct an RNA replicated by the SFV-replicase. Since a simple domain swapping of 5′-terminal regions between a VEEV and an SFV saRNA would most likely lead to a non-functional vector, the complete 5′-regulatory region of VEEV or VEEV-TC83 was fused to an SFV saRNA. More exactly, the SFV untranslated region comprising CSE1 was deleted and the VEEV sequences were fused directly upstream of the SFV-replicase start codon. The resulting VEEV/SFV-chimeric saRNA has a 5′ VEEV-derived regulatory region but encodes an SFV-replicase and SFV-derived CSE3 (SGP) and CSE4 (3′ CSE) (FIG. 10A). These vectors were in vitro transcribed to non-modified or modified RNA having increasing rates of 1mY instead of uridine, from 0% to 100%. It was observed that increasing modification reduced the replication ability of the chimeric saRNA in K562 cells, unless it contained the TC-83 specific 5′-mutation (FIG. 10B). When lipofected into interferon-competent primary fibroblasts, the TC-83 variant of the chimeric saRNA was better expressed and resistant towards modification, while the non-TC83 counterpart displayed reduced expression upon 100% modification (FIG. 10C). The inhibition of replication by the presence of 1mY was less pronounced than in K562 cells, which was most likely due to a bias resulting from reduced innate immune stimulation of the modified saRNA. Overall, these data show that a 5′-regulatory region with increased resistance to nucleotide modification in a trans-replicating RNA was successfully transposed onto a self- or cis-replicating RNA.

A comparable experiment was performed with similar chimeric VEEV/SFV saRNA encoding for GFP. K562 and HFF cells were lipofected and GFP-expression assessed the next day to determine transfection rates of the cells. The transfection rates of the VEEV/SFV saRNA with the 5′ CSE of TRD decreased with increasing 1mY incorporation, while the 5′ CSE of TC-83 provided resistance towards 1mY modification, as well as an overall increased transfection rate (FIGS. 10D, 10E). These results were confirmed with constructs encoding firefly luciferase.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Example 11

### Material and Methods

Immunizations: 6-8 week old female BALB/c mice were kept in specific-pathogen-free conditions in accordance to the FELASA recommendations and in compliance with the German animal welfare act and Directive 2010/63/EU. All work was approved by the the Landesuntersuchungsamt Rhineland-Palatinate under reference number G-20-8-043. Mice were immunized intramuscularly (i.m.) into the anterior tibialis with 100 ng LNP formulated mixture of replicase-mRNA and TR coding for HA from Influenza virus A/California/7/2009 as antigen. Mice were immunized two times with 28 days apart. The composition of RNA for each injection was 78 ng of SFV-replicase mRNA bearing 1mY and 22 ng of respective TR bearing either U or 1mY. All RNAs were co-transcriptionally capped.

ELISpot Analysis: Splenocytes were isolated on day 56 and ELISpot analysis was performed using the Mabtech Mouse IFN-γ ELISpotPLUS kit. Splenocytes were seeded to pre-coated ELISpot plates and stimulated with the indicated peptide pools overnight in a humidified incubator at 37° C.

| MHC-I specific peptide pool | MHC-II specific peptide pool |
|---|---|
| LYEKVRSQL (SEQ ID NO: 3) | STRIYQILAIYSTVA (SEQ ID NO: 9) |
| CPHAGAKSF (SEQ ID NO: 4) | KYVKSTKLRLATGLR (SEQ ID NO: 10) |
| VEPGDKITF (SEQ ID NO: 5) | GKCPKYVKSTKLRLA (SEQ ID NO: 11) |
| SYINDKGKEV (SEQ ID NO: 6) | AIYSTVASSLVLVVS (SEQ ID NO: 12) |
| FYHKCDNT (SEQ ID NO: 7) | EELREQLSSVSSFER (SEQ ID NO: 13) |
| STASSWSYI (SEQ ID NO: 8) | VKLESTRIYQILAIY (SEQ ID NO: 14) |

Control measurements were performed using an irrelevant peptide pool, medium only or Concanavalin A. Spots were visualized with a biotin-conjugated anti-IFN-γ antibody followed by incubation with streptavidin-alkaline phosphatase (ALP) and 5-bromo-4-chloro-3-indolyl-phosphate/nitroblue tetrazolium (BCIP/NBT) substrate. Plates were scanned using a CTL ImmunoSpot® Analyzer and analyzed by ImmunoCapture V6.3 software. All tests were performed in triplicate and spot counts were summarized as median values for each triplicate.

Antibody analysis: For antibody analysis, blood samples were taken under isoflurane anesthesia on day 56 after the first immunization. Virus neutralization test (VNT) to determine neutralizing antibodies in the serum was performed based on the WHO's Manual for the Laboratory Diagnosis and Virological Surveillance of Influenza (WHO Global influenza Surveillance Network). Serum levels of HA-neutralizing antibodies were quantified by incubating serial dilutions of the mouse sera for 2 h with 100 tissue culture infection dose 50 (TCID₅₀) of active Cf4/H1N1 Influenza virus and then applied onto a confluent MDCK cell monolayer in 96 well plates. Upon incubation for 3 days with MDCK, cell culture supernatant was harvested and mixed 1:2 with 0.5% chicken red blood cells (RBC; Lohmann Tierzucht, Cuxhaven, Germany). RBC agglutination was visually observed in round-bottom 96-well-plates and the VN serum titer was recorded as the inverse of the lowest dilution that inhibited agglutination (VNT-titer/50 μl serum).

ELISA: Recombinant Cf4/H1N1-HA protein (Life Sciences, Idstein, Germany) was biotinylated utilizing the EZ-Link Sulfor-NHS-LC-biotinylation kit according to supplier's protocol (Thermo Fisher Scientific, Germany). 96 well Streptavidin plates (VWR, Darmstadt, Germany) were coated with the biotinylated HA protein at 4° C. overnight. Upon washing and blocking, serum samples were screened for HA-specific antibodies by incubation on plates for 1 h at 37° C. After incubation and including assay controls, plates were incubated with HRP-labeled secondary anti-mouse IgG antibody for another 45 min at 37° C. before TMB one substrate (BIOTREND, Cologne, Germany) was applied. Colorimetric detection was monitored and optical density read at 450 nm calculated to a wavelength reference of 620 nm (λ450-620 nm).

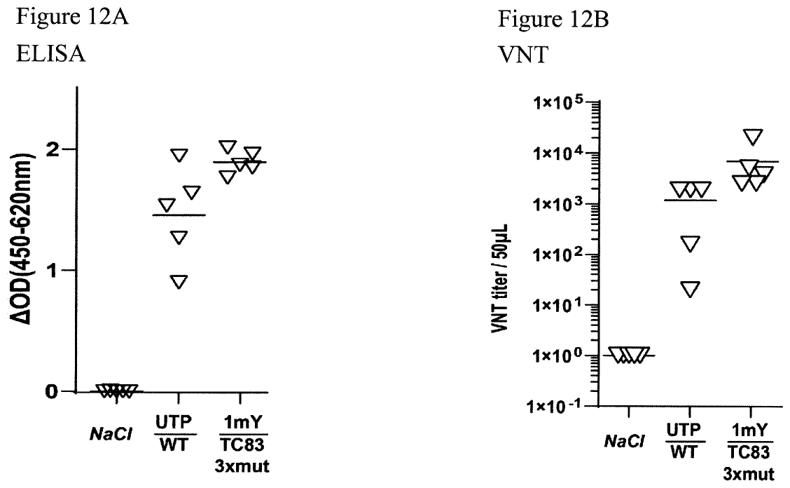

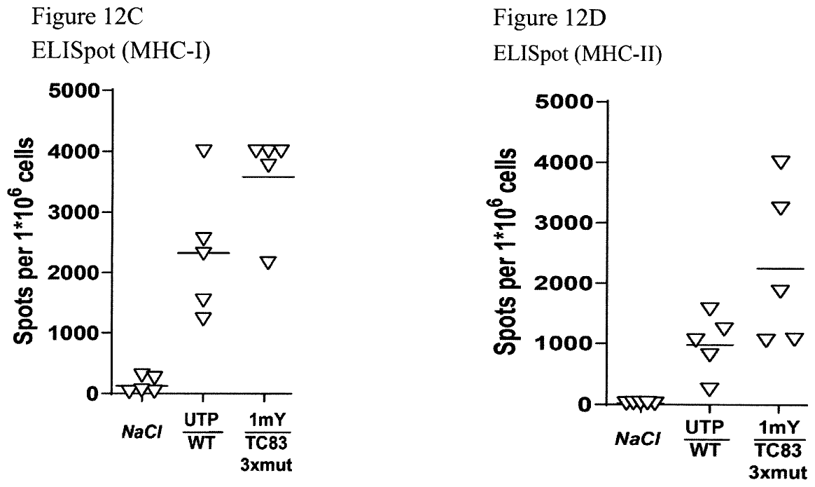

Results: To address the question whether the usage of in vitro evolved TR with TC-83 specific 5′ mutation and the three mutations abbreviated as 3xMut (TC-83+3xMut) improves the potency of modified replicating RNA as vaccine vector, Hemagglutinin gene of Influenza A virus was inserted into TR-TC83+3xMut and in the parental TR (WT). TR-TC83+3xMut RNA was generated in the presence of 1mY-TP whereas for WT TR mRNA UTP was used. Each TR was combined in equimolar ratio with SFV-replicase mRNA bearing 1mY and formulated in LNP. For i.m. vaccinations in prime boost regimen, a total dose of 100 ng of each mixture per injection was used. After 56 days, blood was collected to obtain sera to determine humoral immunity and splenocytes were isolated to assess cell-mediated immunity. The endpoint titration of HA-specific antibodies measured by ELISA revealed greater mean concentration of circulating HA-specific IgG in the peripheral blood of mice vaccinated with TR-TC83+3xMut containing 1mY compared to the WT-TR containing U (FIG. 12A). Increased antibody concentrations were reflected by increased mean virus neutralizing titers (VNT) (FIG. 12B). T cell immunity was also increased as shown by ELISpot analysis specific for (FIG. 12C) CD8+ T cells and (FIG. 12C) CD4⁺ T cells.

In summary, the data show that the immunization with the 1mY modified TR-TC83+3xMut enabled the usage of 100% 1mY modified replicating RNA as vaccine vector without losing the ability to induce immunogenicity.

### Example 12

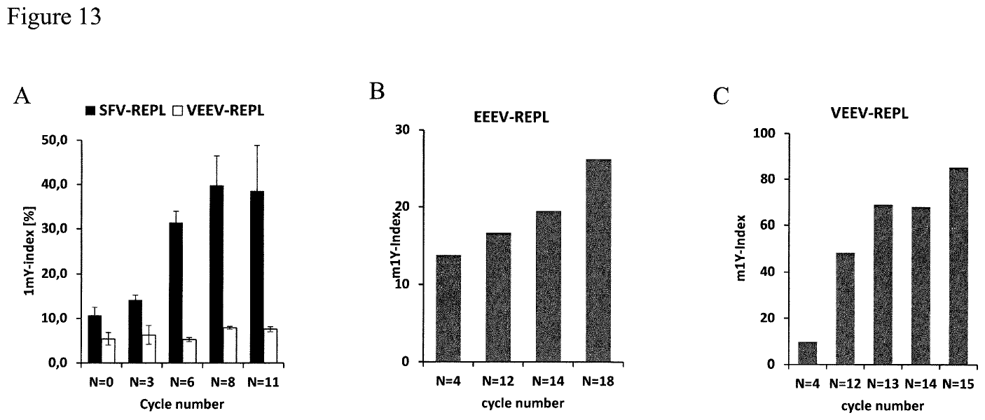

The in vitro evolution procedure that was performed for 11 cycles using the SFV-replicase resulted in a TR that was robustly replicated when modified with 1mY (FIG. 4B). To assess whether this increased compatibility for the modification was specific for the SFV-replicase, modified and unmodified TRs amplified from the cDNA of transfected cells after several cycles of the in vitro evolution were co-transfected with the SFV-replicase or with the VEEV replicase (FIG. 13A). When using the SFV-replicase, the successive adaptation to 1mY modification is reflected by a successive 1mY index alongside the cycles of in vitro evolution, which we did not observe when using the VEEV-replicase to replicate the same evolved TRs. This suggests an adaptation that is effective in conjunction with the replicase used during the evolution process. The in vitro evolution process, however, should be applicable with replicases of other alphaviral species as well. Therefore, we aimed to assess whether a TR identified using the SFV-replicase (having acquired mutation 67, 245 & 248, called 3xmut) would evolve towards a better compatibility with 1mY using the replicase of VEEV (Venezuelan equine encephalitis virus) or EEEV (Eastern equine encephalitis virus). TR-3xmut was thus subjected to further cycles of the described in vitro evolution, using the VEEV and the EEEV replicase-coding mRNA instead of SFV-replicase. The cycle count was reset, and after each round of in vitro evolution we determined the 1mY-index based on CD90.1 selection marker expression with unmodified and modified TR. We observed that the 1mY index increased the more cycles were performed using (FIG. 13B) the EEEV replicase, or (FIG. 13C) the VEEV-replicase. Thus, the in vitro evolution process described herein results in replicating TRs adapted to 1mY regardless of the replicase used.

We claim:

1. A method for identifying a sequence change in a replicable RNA (rRNA) comprising a modified nucleotide (modified rRNA) that at least partially restores or increases a function of the modified rRNA, the method comprising:
(a) transfecting cells expressing an RNA-dependent RNA polymerase (replicase) with a modified rRNA encoding a gene of interest; and
(b) identifying a sequence change within the rRNA molecules encoding the gene of interest present in the transfected cells expressing the gene of interest.

2. The method according to claim 1, wherein the sequence change is determined relative to the sequence of the modified rRNA transfected in step (a).

3. The method according to claim 1 or 2, wherein step (a) further comprises, after transfecting, isolating rRNA molecules encoding the gene of interest from the transfected cells expressing the gene of interest to provide isolated rRNA molecules.

4. The method according to claim 3, wherein step (a) further comprises a step of transfecting cells expressing the replicase with a subsequent generation of rRNA molecules encoding the gene of interest and comprising the modified nucleotide, which subsequent generation is provided by replicating the isolated rRNA molecules.

5. The method according to claim 4, which method further comprises repeating the steps of claims 3 and 4.

6. The method according to claim 5, wherein the step is repeated n times.

7. The method according to claim 6, wherein n is an integer of at least 1.

8. The method according to any one of claims 4 to 7, wherein replicating the isolated rRNA molecules comprises (i) reverse-transcribing the isolated rRNA molecules encoding the gene of interest to form DNA molecules that are capable of being in vitro transcribed; and (ii) in vitro transcribing the DNA molecules in the presence of the same modified nucleotide to produce a subsequent generation of modified rRNA molecules comprising the modified nucleotide and encoding the gene of interest.

9. The method according to any one of claims 1 to 8, further comprising the steps:
(c) incorporating at least one identified sequence change into the modified rRNA of step (a);
(d) transfecting cells expressing the replicase with the modified rRNA produced in step (c); and
(e) identifying a sequence change within the rRNA molecules isolated from the transfected cells expressing the gene of interest produced in step (d).

10. The method according to any one of claims 1 to 9, wherein the number of modified nucleotides in the modified rRNA is at least 10%, 20%, 30%, 40%, 50%, 60%, 70%, 80%, 90% or 100% of the total number of nucleotides in the rRNA.

11. The method according to claim 10, wherein the number of modified nucleotides in the modified rRNA is at least 30% of the total number of nucleotides in the rRNA.

12. The method according to claim 10, wherein the number of modified nucleotides in the modified rRNA is at least 50% of the total number of nucleotides in the rRNA.

13. The method according to any one of claims 1 to 12, wherein the modified nucleotide is a modified uridine residue, a modified adenine residue, a modified guanine residue, a modified cytosine residue, or any combination of two of more of the foregoing.

14. The method according to any one of claims 1 to 13, wherein the modified nucleotide is a modified uridine.

15. The method according to claim 14, wherein the modified uridine is pseudouridine.

16. The method according to claim 15, wherein the pseudouridine is N1-methyl-pseudouridine.

17. The method according to claim 16, wherein the number of N1-methyl-pseudouridine residues in the in vitro transcribed modified rRNA is at least 10%, 20%, 30%, 40%, 50%, 60%, 70%, 80%, 90% or 100% of the total number of uridine residues.

18. The method according to claim 17, wherein the number of N1-methyl-pseudouridine residues in the in vitro transcribed modified rRNA is at least 30% of the total number of uridine residues.

19. The method according to claim 17, wherein the number of N1-methyl-pseudouridine residues in the in vitro transcribed modified rRNA is at least 50% of the total number of uridine residues.

20. The method according to any one of claims 1 to 19, wherein the replicase is derived from a self-replicating RNA virus, preferably an alphavirus.

21. The method according to claim 20, wherein the alphavirus is SFV or VEEV or EEEV or Sindbis virus.

22. The method according to any one of claims 1 to 21, wherein the rRNA comprises a 5′ regulatory region that has no start codons.

23. The method according to any one of claims 1 to 22, wherein the replicase and the regulatory sequences in the rRNA required by the replicase for replication are derived from the same alphavirus or from different alphaviruses.

24. The method according to any one of claims 1 to 23, wherein the encoded gene of interest is a cell surface expressed protein or luciferase or GFP.

25. The method according to any one of claims 1 to 24, wherein the expression of the replicase in the cells is transient expression or constitutive expression.

26. The method according to any one of claims 1 to 25, wherein the cells are transfected with a nucleic acid molecule encoding the replicase prior to, concurrently with, or after transfection of the rRNA, preferably wherein the nucleic acid molecule is an RNA.

27. The method according to any one of claims 1 to 26, wherein step (a) comprises transfecting cells with a self-amplifying RNA encoding a gene of interest, which self-replicating RNA comprises at least one modified nucleotide.

28. The method according to any one of claims 1 to 27, wherein the function of the rRNA that is partially restored or increased is the ability to be replicated by the replicase and/or is the ability to be translated.

29. The method according to any one of claims 1 to 28, wherein the function is restored by at least 10% or is increased by at least 10%.

30. The method according to any one of claims 1 to 29, wherein the step of identifying the sequence change comprises sequencing at least a portion of the rRNA molecules.

31. The method according to claim 30, wherein the portion of the rRNA that is sequenced is the 5′ regulatory region.

32. The method according to claim 30, wherein the portion of the rRNA that is sequenced is the 3′ regulatory region.

33. The method according to any one of claims 30 to 32, wherein the sequence change is determined by analyzing sequence-specific read frequencies generated by sequencing.

34. The method according to any one of claims 1 to 33, wherein the method is carried out to increase the replication efficiency of a self-amplifying RNA comprising at least one modified nucleotide.

35. The method according to any one of claims 1 to 34, wherein the method further comprises modifying the nucleotide sequence of an rRNA or self-replicating RNA comprising the same modified nucleotide by incorporating at least one identified sequence change that partially restores or increases the function of the rRNA or self-replicating RNA comprising the modified nucleotide.

36. A replicable RNA (rRNA) molecule comprising at least one modified nucleotide and obtained by the method according to any one of claims 1 to 35.

37. A replicable RNA (rRNA) having a sequence change that at least partially restores or increases a function of the modified rRNA obtained by a method comprising:
(a) transfecting cells expressing an RNA-dependent RNA polymerase (replicase) with a modified rRNA encoding a gene of interest; and
(b) identifying a sequence change within the rRNA molecules encoding the gene of interest present in the transfected cells expressing the gene of interest.

38. A replicable RNA (rRNA) molecule comprising a modified 5′ regulatory region of a self-replicating RNA virus, which modified regulatory region comprises a point mutation at one or more of positions 67, 244, 245, 246, 248 of the 5′ regulatory region (SEQ ID NO: 2).

39. The rRNA molecule according to claim 38, wherein the self-replicating RNA virus is an alphavirus.

40. The rRNA molecule according to claim 38 or 39, wherein the rRNA molecule comprises a point mutation at position 67 and at one or more of positions 244, 245, 246, 248 of the 5′ regulatory region (SEQ ID NO: 2).

41. The rRNA molecule according to any one of claims 38 to 40, wherein the 5′ regulatory region further comprises a point mutation at position 4 of the 5′ regulatory region (SEQ ID NO: 2).

42. The rRNA molecule according to any one of claims 38 to 41, wherein the point mutation is at position 67.

43. The rRNA molecule according to any one of claims 38 to 41, wherein the point mutation is at positions 67 and 244.

44. The rRNA molecule according to any one of claims 38 to 41, wherein the point mutation is at positions 67 and 246.

45. The rRNA molecule according to any one of claims 38 to 41, wherein the point mutation is at positions 67 and 248.

46. The rRNA molecule according to any one of claims 38 to 41, wherein the point mutation is at positions 67, 245 and 248.

47. The rRNA molecule according to any one of claims 38 to 41, wherein the point mutation is at positions 4 and 67.

48. The rRNA molecule according to any one of claims 38 to 41, wherein the point mutation is at positions 4, 67 and 244.

49. The rRNA molecule according to any one of claims 38 to 41, wherein the point mutation is at positions 4, 67 and 246.

50. The rRNA molecule according to any one of claims 38 to 41, wherein the point mutation is at positions 4, 67 and 248.

51. The rRNA molecule according to any one of claims 38 to 41, wherein the point mutation is at positions 4, 67, 245 and 248.

52. The rRNA molecule according to any one of claims 38 to 51, wherein the point mutation is G4A, A67C, G244A, C245A, G246A, or C248A.

53. The rRNA molecule according to any one of claims 36 to 52, wherein the self-replicating RNA virus is SFV or EEEV or VEEV or Sindbis virus.

54. The rRNA molecule according to any one of claims 36 to 53, further comprising one or more coding regions.

55. The rRNA molecule according to any one of claims 36 to 54, which encodes a gene of interest.

56. The rRNA molecule according to claim 55, wherein the encoded gene of interest is an antigen or a therapeutic protein or a nucleic acid, preferably a tumor, viral, bacterial, or fungal antigen, or an allergen.

57. The rRNA molecule according to any one of claims 36 to 56, which encodes an RNA-dependent RNA polymerase (replicase), preferably an alphaviral replicase.

58. The rRNA molecule according to any one of claims 36 to 57, comprising a 5′ regulatory region and which encodes a replicase, wherein the 5′ regulatory region and the encoded replicase are derived from the same self-replicating RNA virus, preferably the same alphavirus.

59. The rRNA molecule according to any one of claims 36 to 57, comprising a 5′ regulatory region and which encodes a replicase, wherein the 5′ regulatory region and the encoded replicase are derived from different self-replicating RNA viruses.

60. The rRNA molecule according to any one of claims 36 to 59, which is a self-amplifying RNA.

61. The rRNA molecule according to any one of claims 36 to 60, which is an in vitro transcribed rRNA.

62. The rRNA molecule according any one of claims 36 to 61, wherein the rRNA comprises at least one modified nucleotide.

63. The rRNA molecule according to claim 62, wherein the modified nucleotide is N1-methyl-pseudouridine.

64. The rRNA molecule according to claim 63, wherein all uridine residues are N1-methyl-pseudouridine residues.

65. A DNA molecule encoding the rRNA molecule according to any one of claims 36 to 64.

66. The rRNA molecule according to any one of claims 36 to 64 or the DNA molecule of claim 65, wherein the rRNA or DNA molecule is linear or circular.

67. The rRNA or DNA molecule according to any one of claims 36 to 66, which is formulated with lipids.

68. A pharmaceutical composition comprising the rRNA or DNA molecule according to any one of claims 36 to 67 and a pharmaceutically acceptable carrier or excipient.

69. A rRNA or DNA molecule according to any one of claims 36 to 67 or a pharmaceutical composition according to claim 68 for use in therapy.

70. A method for raising an immune response in a subject comprising administering an rRNA molecule encoding an antigen to the subject, and which rRNA molecule comprises a modified 5′ regulatory region of a self-replicating RNA virus, preferably an alphavirus, which modified regulatory region comprises a point mutation at one or more of positions 67, 244, 245, 246, 248 (of SEQ ID NO: 2).

71. A method for treating cancer in a subject comprising administering an rRNA molecule encoding a tumor antigen to the subject, and which rRNA comprises a modified 5′ regulatory region of a self-replicating RNA virus, preferably an alphavirus, which modified regulatory region comprises a point mutation at one or more of positions 67, 244, 245, 246, 248 (of SEQ ID NO: 2).

72. A method for providing gene function to a subject lacking such gene function comprising administering an rRNA molecule encoding a gene product (therapeutic protein) to the subject to provide the gene function, and which rRNA molecule comprises a modified 5′ regulatory region of self-replicating RNA virus, preferably an alphavirus, which modified regulatory region comprises a point mutation at one or more of positions 67, 244, 245, 246, 248 (of SEQ ID NO: 2).

73. The method according to any one of claims 70 to 72, wherein the rRNA molecule comprises a point mutation at position 67 and at one or more of positions 244, 245, 246, 248 of the 5′ regulatory region (SEQ ID NO: 2).

74. The method according to any one of claims 70 to 73, wherein the rRNA molecule is a self-amplifying RNA molecule comprising at least one modified nucleotide, preferable N1-methyl-pseudouridine.
